(19) United States
(12) Patent Application Publication
(10) Pub. No.: US 2007/0099822 A1
(43) Pub. Date: May 3, 2007

(54) Regulation of tissue mineralization and phosphate metabolism by asarm peptides

(76) Inventor: Peter S.N. Rowe, Prairie Village, TX (US)

Correspondence Address: NEEDLE & ROSENBERG, P.C., SUITE 1000, 999 PEACHTREE STREET, ATLANTA, GA, 30309-3915, US

(21) Appl. No.: 10/567,938

(22) PCT Filed: Sep. 20, 2004
(86) PCT No.: PCT/US04/30530

§ 371 Date: Jul. 13, 2006

(30) Foreign Application Priority Data
Sep. 19, 2003 (US) 60504044

(51) Int. Cl. A61K 38/17; C07K 14/705

(52) U.S. Cl. 514/7; 530/352

(57) ABSTRACT

Disclosed are methods and compositions for treating diseases and conditions involving abnormal serum phosphorus levels and diseases and conditions involving abnormal mineralization levels of bone, teeth, and soft tissues. Also disclosed are methods and compositions for treating cancer and cancer metastasis.

### CROSS-REFERENCE TO RELATED APPLICATIONS

This application claims benefit of priority from U.S. Provisional Application No. 60/504,044, filed Sep. 19, 2003, which is herein incorporated by reference in its entirety.

### STATEMENT REGARDING FEDERALLY SPONSORED RESEARCH

The disclosed subject matter was made with government support under Grants RO-1 AR51598-01 and 1R0-3 DE015900-01 awarded by the National Institute of Arthritis and Musculoskeletal Skin Diseases and the National Institute of Dental and Craniofacial Research, respectively. The US government has certain rights in the invention.

### FIELD

The disclosed subject matter relates generally to methods and compositions for treating diseases and conditions involving abnormal serum phosphorus levels and diseases and conditions involving abnormal mineralization levels of bone, teeth, and soft tissues. The disclosed subject matter also relates generally to methods for treating cancer.

### BACKGROUND

Phosphate is essential for bone mineralization, muscle contraction, and many other cellular processes. Phosphate is normally absorbed from food via a sodium-dependent phosphate cotransporter in the intestine. Phosphate is lost passively from the blood into the glomerular filtrate, and is actively reabsorbed via a sodium-dependent phosphate cotransporter in the proximal tubule of the kidney. Skeletal mineralization is dependent on the regulation of phosphate and calcium levels in the body; therefore, any disturbances in phosphate-calcium homeostasis can have severe repercussions on bone integrity and other body structures and functions.

Bone mineralization is also affected by vitamin D, which plays a role in maintaining phosphate balance. For example, vitamin D deficiency causes rickets in children and osteomalacia in adults. Both conditions are characterized by failure of mineralization of the osteoid (bone matrix). There are also several non-dietary conditions which can lead to rickets, including X-linked vitamin D-resistant hypophosphatemic rickets (HYP), hereditary hypercalciuria with hypophosphatemic rickets (HHRH), and oncogenic hypophosphatemic osteomalacia (OHO). Thus, a number of familial diseases have been characterized that result in disorders of phosphate uptake and bone mineralization.

The PHEX gene has been found to be defective in patients with X-linked hypophosphatemic rickets (HYP). PHEX is a type II glycoprotein and a member of a family of Zn metalloendopeptidases known as M13. PHEX has been proposed to function by processing a factor that plays a role in phosphate homeostasis and skeletal mineralization.

Oncogenic hypophosphatemic osteomalacia (OHO), the adult equivalent of rickets, has many similarities to HYP with an overlapping pathophysiology, but different primary defects. A key feature of tumor-acquired osteomalacia is softening of the bones, which become distorted, resulting in bow-legs and other associated changes reminiscent of familial rickets. Low serum phosphate, and abnormal vitamin-D metabolism are also key features shared with HYP. Tumor acquired osteomalacia is rare, and the tumors are mainly of mesenchymal origin, although a number of different tumor types have also been reported. Surgical removal of the tumor(s) when possible, results in the disappearance of disease symptoms and bone healing, suggesting the role of a circulating phosphaturic factor(s) in the pathogenesis of the disease.

A broad variety of diseases and conditions involving abnormal phosphate metabolism include diseases and conditions associated with hyperphosphatemia (such as renal insufficiency, which can result in ectopic calcification of soft tissues; in particular, coronary and aortic calcification is a major problem in hemodialysis patients), diseases and conditions associated with hypophosphatemia (such as rickets, osteomalacia, severe diarrhea, starvation, Cushing syndrome, intestinal malabsorption syndromes, kidney transplantation, acute renal failure, end-stage renal disease, chronic alcoholism and alcohol withdrawal, severe thermal burns, acute malaria, and various drug therapies, which can result in skeletal muscle injury, cardiomyopathy and cardiac arrhythmia, respiratory insufficiency, and bone demineralization), and osteoporosis, for example.

In view of the broad variety of diseases and conditions that involve abnormalities in phosphate metabolism, there is a need in the art for new methods and compounds for regulating patients' phosphate levels in order to treat such diseases and conditions.

### SUMMARY

MEPE is a secreted phosphoglycoprotein that promotes renal phosphate excretion and inhibits bone mineralization. The disclosed subject matter is based on the discovery, as described herein, that cleavage of MEPE in vivo releases a carboxy-terminal peptide (the “ASARM peptide”) that is responsible for the MEPE activity in vivo. As described herein, administration of isolated ASARM peptides can be used to treat various diseases and conditions involving either hyperphosphatemia or hypophosphatemia. Moreover, it has now been discovered that MEPE can be administered to inhibit tumor growth in patients with cancer. Further, MEPE and/or specific MEPE-peptides (ASARM-peptide(s) for example) can be used to directly prevent tumor-bone metastasis.

In a first aspect, the disclosed subject matter features a method of treating hyperphosphatemia in a subject, including administering an isolated ASARM peptide to the subject using a dosage regimen that decreases serum phosphate levels, thereby treating hyperphosphatemia in the subject.

The hyperphosphatemia can be associated with, e.g., end stage renal disease, renal osteodystrophy, chronic renal disease, renal toxicity, calcification of kidneys, kidney stones, calcification of arteries, or atherosclerotic lesions.

In a second aspect, the disclosed subject matter features a method of treating hypophosphatemia in a subject, wherein the subject displays a normal level of endogenous ASARM peptide, including administering an isolated ASARM peptide to the subject using a dosage regimen that increases serum phosphate levels, thereby treating hypophosphatemia in the subject.

For example, subjects displaying a normal level of endogenous ASARM peptide would not have X-linked hypophosphatemic rickets or oncogenic hypophosphatemic osteomalacia.

In various examples of the second aspect of the disclosed subject matter, the hypophosphatemia can be associated with hereditary hypophosphatemic rickets with hypercalciuria, autosomal dominant hypophosphatemic rickets, receptor defect rickets, familial rickets, vitamin-D dependent rickets type-1, defective 25-hydroxylase, Fanconi syndrome, oncogenous syndrome, osteodystrophy, Pagets disease, or metaphyseal dysplasia.

In a third aspect, the disclosed subject matter features a method of treating or inhibiting osteoporosis in a subject, including administering an isolated ASARM peptide to the subject in a dosage regimen that decreases bone resorption, thereby treating or inhibiting osteoporosis in the subject.

In a fourth aspect, the disclosed subject matter features a method of treating a subject with a disease involving pathologically elevated levels of endogenous ASARM peptide, including inhibiting ASARM peptide activity in the subject, thereby treating the subject with a disease involving pathologically elevated levels of endogenous ASARM peptide.

For example, the disease involving pathologically elevated levels of endogenous ASARM peptide can be X-linked hypophosphatemic tickets or oncogenic hypophosphatemic osteomalacia.

In a fifth aspect, the disclosed subject matter features a method of treating or inhibiting ectopic tissue mineralization in a subject, including administering isolated ASARM peptide such that ectopic tissue mineralization is inhibited, thereby treating or inhibiting ectopic tissue mineralization in the subject.

For example, the ectopic mineralization can be due to periodontal disease or kidney disease.

In a sixth aspect, the disclosed subject matter features a method of inhibiting tumor growth and/or tumor metastasis to bone and/or soft tissue in a subject, including administering MEPE and/or MEPE peptides and/or ASARM-peptide to the subject, thereby inhibiting tumor growth and/or tumor metastasis to bone and/or soft tissue in the subject.

For example, the tumor can be of mesenchymal, epithelial, endothelial, neuroectodermal origin, or hematologic cancer cells (cancer of the plasma as in multiple myeloma).

In a seventh aspect, the disclosed subject matter features a method of preventing cancer cell metastasis to bone and/or soft tissue in a subject with cancer, including administering ASARM-peptide and/or MEPE and/or MEPE peptide to the subject, thereby preventing cancer cell metastasis to bone and/or soft tissue in the subject.

For example, the subject can have cancer with multiple myeloma, breast cancer, prostrate cancer, and/or other cancers of similar malignant predisposition.

In an eight aspect, the disclosed subject matter features a method of identifying a subject with an increased likelihood of having or developing a disease or condition involving abnormal phosphate metabolism, including detecting an abnormal level of endogenous ASARM peptide in the subject as compared to a control, wherein an abnormal level of endogenous ASARM peptide in the subject identifies a subject with an increased likelihood of having or developing a disease or condition involving abnormal phosphate metabolism.

For example, the abnormal phosphate metabolism can result in hypophosphatemia (e.g., but not limited to, from X-linked hypophosphatemic rickets or tumor-induced osteomalacia). In such cases, the abnormal phosphate metabolism can be characterized by decreased bone or tooth mineralization or increased bone or tooth resorption, compared to a normal subject, and the abnormal phosphate metabolism can cause osteoporosis, periodontal disease, or dental caries.

In another example of the eight aspect of the disclosed subject matter, the subject can have hyperphosphatemia. In such cases, the abnormal phosphate metabolism can cause periodontal disease, kidney stones, renal calcification, blood vessel calcification, or other soft tissue mineralization.

In a ninth aspect, the disclosed subject matter features a pharmaceutical composition comprising an isolated ASARM peptide and a pharmaceutically acceptable carrier.

In a tenth aspect, the disclosed subject matter features a method of identifying a compound for treating a disease involving a pathologically elevated level of ASARM peptide, including (a) contacting a sample comprising an ASARM peptide with the compound, and (b) detecting a decrease in biological activity of ASARM peptide, whereby an decrease in biological activity of ASARM peptide indicates a compound for treating a disease involving a pathologically elevated level of ASARM peptide.

In all of the above aspects of the disclosed subject matter, the isolated ASARM peptide can be unphosphorylated or at least one serine residue of the isolated ASARM peptide can be phosphorylated. For example, all of the serine residues of the isolated ASARM peptide can be phosphorylated if desired.

Additional advantages of the disclosed subject matter will be set forth in part in the description which follows, and those skilled in the art will recognize that other and further changes and modifications can be made thereto without departing from the spirit of the invention. The advantages of the disclosed subject matter will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

The accompanying drawings, which are incorporated in and constitute a part of this specification, illustrate several aspects described below.

FIGS. 1a-1b shows key features and localization of the acidic-serine-aspartate-rich-MEPE associated motif (ASARM-peptide and motif). FIG. 1a is a scheme showing the carboxy-terminal residues of MEPE in man (RDDSSESSDSGSSSESDGD; SEQ ID NO. 1), mouse (RDSSESSSSGSSSESHGD; SEQ ID NO. 2), and rat (RDSSESSSSGSSSESSGD; SEQ ID NO. 3) respectively. The ASARM-motif is highly conserved in man, macaque-monkey (REDSSESSDSGSSSESDGD; SEQ ID NO. 16), mouse and rat and is localized to the last COOH-terminal 18 amino acid residues of the large approximately 500 residue MEPE proteins as depicted in the scheme. Cathepsin-B (an osteoblast protease) specifically cleaves MEPE at the COOH-terminus releasing ASARM-peptide. The cathepsin-B cleavage site does not occur elsewhere in MEPE and is highly conserved between species. Moreover, the ASARM-peptide is uniquely resistant to many proteases (trypsin, papain, proteinase K, carboxypeptidases, tryptase etc). The ASARM-motif is found in members of the SIBLNG protein family (MEPE, DMP-1, osteopontin, DSPP) and in osteopontin occurs in the mid-region of the molecule and also salivary statherin. The dark boxes represent the position of the ASARM-motif in MEPE and osteopontin and the number of amino acid residues for each respective protein is indicated.

FIG. 2 is a scheme illustrating a proposed role and molecular mechanism for PHEX, MEPE and the ASARM-peptide in mineralization. Cathepsin-B is expressed in the osteoblast together with PHEX and MEPE. NEP, ECEL-1/DINE and cathepsin-D and MEPE are up regulated in Hyp osteoblasts that have defective PHEX. PHEX protects MEPE from cathepsin-B and protease cleavage (possibly by sequestration on the cell surface) and prevents release of the ASARM-peptide. Thus, in rickets due to defective PHEX, increased levels of ASARM-peptide lead to inhibition of mineralization. Also, the ASARM-peptide is resistant to a vast range of proteases due to its unusual sequence. The MEPE ASARM-peptide (NH₂-FSSRRRDDSSESSDSGSSSESDGD-COOH; SEQ ID NO: 4) inhibits mineralization in-vivo.

FIGS. 3a-3b show SDS-polyacrylamide electrophoretic separation and Western blotting of purified insect expressed full-length human-MEPE (Hu-MEPE). FIG. 3a shows a Coomassie stained gel with three lanes containing bovine serum albumin (BSA), a single lane of standard protein molecular weight markers (Ma) and two lanes containing MEPE. FIG. 3b shows the corresponding PVDF membrane-blot containing immobilized proteins screened with mid-region (RGD-peptide) MEPE polyclonal (see methods section). Confirmation of MEPE identity was achieved by N-terminal amino-acid sequence analysis of the excised PVDF blot region corresponding to the positive Western-band. Purified protein contained N-terminal amino acid residues APTFQ (SEQ ID NO. 7) confirming cleavage of predicted nascent-MEPE signal-peptide by the sf9 insect cells.

FIGS. 4a-4c show that dose-dependent in-vitro inhibition of ³³PO₄-uptake is induced by Hu-MEPE in primary human proximal-tubule epithelial cells (RPTEC) and a human renal cell-line (Hu-CL8). FIGS. 4a-4b graphically illustrate the inhibition using lineweaver-burke plots (inverse percentage rate of inhibition against the inverse MEPE concentration (ng/mL)). The Hu-CL8 cell line (FIG. 4a) and human RPTEC primary renal-cells (FIG. 4b) have similar Kₘ values (23.8 ng/mL and 27.39 ng/mL respectively) but differing Vₘₐₓ values (9.1% and 53.4% respectively). FIG. 4c is a histogram illustrating Hu-MEPE and PTH dose-dependent inhibition observed with RPTEC cells in-vitro: normal (buffer solvent), MEPE-25 (25 ng/mL), MEPE-50 (50 ng/mL), MEPE-100 (100 ng/mL), PTH-10 (10 ng/mL), PTH-100 (100 ng/mL). Differences in FIG. 4c were assessed statistically by the use of Newman-Keuls multiple comparison equations after one-way analysis of variance (non-parametric). A p value of less than 0.05 was considered significant. The standard error of the mean (SEM) was used as a representative measure of how far the sample mean differed from the true population mean (see bars). For FIGS. 4a-4c each data point contained N=12 and N=15 replicates for MEPE and PTH respectively.

FIGS. 5a-5c show that MEPE and PTH induce hypophosphatemia and increase the fractional excretion of phosphate (FEP) when administered intraperitoneally (i.p.). FIG. 5a is a histogram of mean (±SEM) serum phosphate measured after 31 hours and four bolus i.p. injections of MEPE40 (40 μg/kg/30-hours), MEPE400 (400 μg/kg/30-hours) and PTH (80 μg/kg/30-hours). FIGS. 5b-5c are histograms illustrating the changes in FEP after 7-hours (3 bolus injections i.p.) and 31-hours (4 bolus injections i.p.). Differences were assessed statistically by the use of Newman-Keuls multiple comparison equations after one-way analysis of variance (non-parametric). A p value of less than 0.05 was considered significant. Each group contained N=7 animals.

FIGS. 6a-6c show that Hu-MEPE and MEPE-ASARM-peptide (CFSSRRRDDSSESSDSGSSSESDGD; SEQ ID NO. 5)) inhibit BMP2-mediated mineralization of mouse osteoblast cell-line 2T3. Wells were stained for mineralization nodule formation using von Kossa and the results after 26 days culture are shown. In FIG. 6a, upper two wells (BMP2), BMP2 (100 ng/mL); middle two wells (control), control cells with no BMP2 or peptide; lower two wells (BMP2 & control peptide), BMP2 (100 ng/mL) with control peptide (CGSGYTDLQERGDNDISPFSGDGQPF; SEQ ID NO. 6) at 300 ng/mL (108.6 picomoles/mL). In FIG. 6b, upper two wells (BMP2 & MEPE 100), Hu-MEPE (100 ng/mL) plus BMP2 (100 ng/mL); middle two wells (BMP2 & MEPE 500), Hu-MEPE (500 ng/mL) plus human-BMP2 (100 ng/mL); lower two wells (BMP2 & MEPE 800), MEPE (800 ng/mL) plus BMP2 (100 ng/mL). Cells in which MEPE was added in the absence of BMP2 were indistinguishable to control cells (FIG. 6a, middle two wells). In FIG. 6c, upper two wells (BMP2 & ASARM 60), BMP2 (100 ng/mL) plus MEPE-ASARM-peptide (CFSSRRRDDSSESSDSGSSSESDGD; SEQ ID NO. 5) at 60 ng/mL (22.7 picomoles/mL); lower two wells (BMP2 & ASARM 300), BMP2 (100 ng/mL) plus MEPE-ASARM-peptide at 300 ng/mL (113.5 picomoles/mL).

FIGS. 7a-7b shows quantification of mineralization inhibition of mouse osteoblast 2T3 cell-line by Hu-MEPE as assessed by von Kossa staining. Concentrations above 100 ng/mL completely inhibited mineralization (see FIG. 6). Thus effects at 10, 100, and 500 ng/mL MEPE are shown after day 13 (FIG. 7a) and day 20 (FIG. 7b) respectively. The mineralized bone-matrix formation of 2T3 cells were quantitated by computer image analysis as previously described (Chen, et al., J Cell Biol 142:295-305, 1998) and the data represent the mean (±SEM) for three samples (see Methods). One-way analysis of variance (ANOVA) non-parametric and Neuman-Keuls multiple comparison confirm highly significant inhibition of BMP2 mediated mineralization by Hu-MEPE (see graph).

FIG. 8 illustrates salivary-statherin and MEPE consensus ASARM-motif: mineralization-inhibition and ancestral genes on chromosome 4. The depicted scheme illustrates the remarkable association of MEPE, DMP-1 and the SIBLINGs to an ancestral mineralization-gene that is also thought to play a key role in phosphate calcium transport in saliva (salivary statherin). Statherin maps to chromosome 4 in the SIBLING/MEPE region and also contains an ASARM-motif. Statherin is a 62 residue peptide with asymmetric charge and structural properties. The upper scheme (FIG. 8a) depicts a clustal alignment of the COOH terminal region of human-DMP-1 human-MEPE, mouse-MEPE and rat-MEPE with human-Statherin. In MEPE the ASARM-peptide is the most distal region of the molecule encompassing the last 17 residues of the COOH-terminus and the region is highlighted with a boxed cartouche labeled MEPE ASARM-peptide (FIG. 8a). In DMP-1 the ASARM-region is also at the carboxy terminus but ends at residue 480 slightly upstream of the distal COOH terminus (protein 513 residues long). The short 62 residue statherin molecule contains an ASARM-motif region as depicted in the diagram and the key residues are highlighted in the consensus string shown at the bottom of FIG. 8a. The boxed cartouche labeled as statherin ASARM-peptide indicates the region of statherin shown to play a biological role in inhibiting spontaneous precipitation of supersaturated salivary calcium and phosphate and maintaining the mineralization dynamics of tooth enamel. As with the MEPE ASARM-peptide a single cathepsin B site is present in statherin that would potentially release the highly charged and phosphorylated aspartate-serine rich statherin ASARM-peptide (indicated by line between statherin arginine (R) residues 29 and 30; FIG. 8a). In Statherin the cathepsin B cleavage site is adjacent and located COOH-terminal to the motif. In MEPE the cathepsin B cleavage site is also adjacent to the ASARM-motif but asymmetrically arranged NH₂-terminal to the motif between the arginine and aspartate. In both cases (MEPE and statherin) cleavage would result in the release of a short phosphorylated aspartate/serine rich acidic peptides of low pI and almost identical physiochemical properties. A feature of the MEPE ASARM-region is the repeat (D)SSES/E sequence. The MEPE ASARM-region is highly homologous to the DMP-1 but the single cathepsin B site in DMP-1 is located further upstream towards the NH₂-terminus (FIG. 8a). FIG. 8b schematically presents the remarkable clustering of MEPE, DMP-1, statherin and other SIBLING genes on chromosome 4. All contain an ASARM-motif in differing structural contexts.

FIG. 9 shows a table of insect-expressed recombinant human-MEPE injected i.p. into mice (N=7) four times and at two different doses (see methods). Three boluses were given over 6-hours and serum/urine collected 1 hour after the third bolus (7 hour time points) and a final fourth bolus was given 30-hours after the first and serum/urine collected 1 hour after the final injection (31 hour time-points). PTH was used as a positive control. Measurements were made on individual (N=7) serum samples except for 1,25-dihydroxy vitamin-D₃ where group samples were pooled and the experiment repeated twice with the same results. Also, group urine samples from respective metabolic cages were pooled and the experiments repeated with the same experimental outcome (see methods). Urine total-protein, K, Cl and glucose were not significantly different between groups and within the normal range. FEP (fractional excretion of phosphate), RPC (renal phosphorus clearance), PEI (phosphate excretion index) calculations are provided in the methods section. Experimentals are coded as PTH-1-34 (PTH at 80 μg/kg/30-h), MEPE40 (40 μg/kg/30-h) and MEPE400 (400 μg/kg/30-h). The results shown represent standard error of the mean (SEM) and the following symbols represent P values calculated using analysis of variance (ANOVA) non parametric and Neuman-Keuls multiple comparison test relative to normal/vehicle groups: P<0.05=*, P<0.01=**, P<0.001=***. The following units are used to express the data: urinary and serum Pi, Ca, creatinine and glucose (mg/dl), urinary/serum Na, K, and Cl (MEQIL), alkaline phosphatase (U/L) and serum 1,25-dihydroxy vitamin-D₃ (pg/mL). The measurements of renal phosphate handling were FEP (%), RPC (μL/min) and PEI (dl/mg).

FIG. 10 is a graph showing serum phosphate levels for four groups of 7 mice that were injected subcutaneously twice a day for three weeks. The first group received vehicle only as a control, the second group received etidronate at 60 mg/kg/day, the third group received ASARM peptide at 1.3 mg/kg/day (“ASARM low”), and the fourth group received ASARM peptide at 4 mg/kg/day (“ASARM high”). Calcein/tetracycline was administered to the animals 4 days and 2 days before the end of the protocol. The vehicle group had a level of 9.62 mg/dl, the etidronate group had a level of 10.04 mg/dl, the low ASARM group had a level 10.60 mg/dl, and the high ASARM group had a level of 10.01 mg/dl.

FIG. 11 is a graph showing alkaline phosphatase levels in serum for the four groups of mice described for FIG. 10 above. Etidronate-treated mice displayed significantly higher levels of serum alkaline phosphatase, whereas both groups of mice treated with ASARM displayed levels similar to that of the control (vehicle).

FIG. 12 is a graph showing serum calcium levels for the four groups of mice as described above for FIG. 10. The mice given etidronate, low ASARM dosage, and high ASARM dosage all displayed lower serum calcium levels than the vehicle (control).

FIG. 13 is a graph showing phosphate excretion in urine from the mice as described for FIG. 10 above. Urine was collected from the groups over a 24-hour period and phosphate was measured. Urinary phosphate excretion was significantly higher in the low ASARM and high ASARM dosage groups than in the etidronate and control groups.

FIGS. 14A-14B are graphs showing the Fractional Excretion of Phosphate (FEP) in two different experiments with differing protocols and amounts of peptide administered (see graphs).

FIG. 15 is a graph showing that both etidronate and ASARM can induce hyperphosphatemia in mice. Three groups of 5 mice each received a subcutaneous injection once a day for 12 days. The first group of mice received an injection of vehicle only. The second group of mice received etidronate at 10 mg/kg/day. The third group of mice received ASARM peptide at 2 mg/kg/day.

FIG. 16 is a graph showing that both etidronate and ASARM peptide induced hypophosphaturia in those mice given etidronate and ASARM peptide under dosage conditions that induce hyperphosphatemia, as described for FIG. 15.

FIG. 17 is a graph showing that urine creatinine was significantly elevated in mice receiving etidronate or ASARM peptide, as described for FIG. 15.

FIG. 18 is a graph showing that Fractional Excretion of Phosphate was significantly lower in the groups that received etidronate or ASARM as described for FIG. 15, compared to the group that received vehicle.

FIG. 19 is a graph showing that tubular reabsorption of phosphate was higher in etidronate and ASARM peptide groups (as described for FIG. 15) compared with the vehicle control group.

FIG. 20 is a graph showing the Phosphate Excretion Index (PEI) of all three mouse groups as described for FIG. 15. Mice receiving etidronate or ASARM display a lower PEI than mice receiving vehicle alone.

FIG. 21 is a graph showing renal phosphate clearance in the three mouse groups as described for FIG. 15. Mice receiving etidronate or ASARM displayed significantly less phosphate clearance than mice receiving vehicle alone.

FIG. 22 is a graph showing urinary phosphate excretion over 24 hours in the three groups of mice as described for FIG. 15. Both etidronate and ASARM peptide groups excreted far less phosphate than did the control group

FIG. 23 is a graph showing serum calcium levels in the three groups of mice as described for FIG. 15.

FIG. 24 is a graph showing urine calcium levels in the three groups of mice as described for FIG. 15.

FIG. 25 is a graph showing serum alkaline phosphatase levels in the three groups of mice as described for FIG. 15.

FIG. 26 shows fluorescent staining in calvariae of representative mice from the three groups of mice as described for FIG. 15.

FIG. 27 is a graph showing that CHO cells expressing MEPE form tumors of dramatically reduced size in nude mice, compared to CHO cells not expressing MEPE.

FIG. 28 is a graph showing that mice containing MEPE-expressing tumors display a decrease in serum phosphate, relative to control mice.

FIG. 29 is a graph showing that mice containing MEPE-expressing tumors display a decrease in serum levels of 1,25-vitamin D3, relative to control mice.

FIG. 30 is a Biacore sensorgram of chip-immobilized MEPE, PHEX and IgG ligands against secPHEX analyte (mobile phase). Ligands (MEPE, PHEX and IgG) were each coupled to a Biacore CM-5 chip at 3500 RU and 2 μM secPHEX analyte flowed through each cell for 6 min. The sec-PHEX, six minute injection-pulse is indicated as a line above the sensorgram.

FIG. 31 is a composite of Biacore sensorgrams of chip-immobilized MEPE ligand against different concentrations of secPHEX analyte (mobile phase). The secPHEX binding to MEPE is dose dependent with a distinct plateau or saturation shown at 10 μM secPHEX. The CM-5 chip was coupled with 3500 RU units of proteins as indicated in FIG. 30. No interaction was detected with control proteins (IgG) on the same chip (see FIG. 30).

FIG. 32 shows binding of secPHEX to MEPE as calculated from data presented in FIGS. 30 and 31. The same coupling of 3500 RU of protein was used. A Bₘₐₓ of 680 RU and an EC₅₀ of 553 nM was computed for secPHEX (analyte) binding to chip immobilized MEPE (ligand). Graph B presents the binding hyperbola of Graph A in linear form by transform plotting of X and Y coordinates as inverse values for secPHEX-MEPE binding.

FIG. 33 are composite PO₄-ASARM-peptide competition Biacore sensorgrams of chip-immobilized MEPE ligand against added secPHEX analyte (mobile phase) in the presence of differing concentrations of PO₄-ASARM-peptide. MEPE ligand was coupled to the chip surface at 6000 RU to increase signal response. PHEX was then injected at a constant 250 nM with a range of concentrations of PO₄-ASARM-peptide.

FIG. 34 shows solution competition of secPHEX-MEPE binding in the presence of PO₄-ASARM-peptide as calculated from data presented in FIG. 33, with MEPE ligand coupled to 6000 RU. A B_{max/inhib} of 68% inhibition of response units (RU) and an apparent K_{Dapp} of 15 nM was computed. Graph B presents the binding hyperbola of Graph A in linear form by transform plotting of X and Y coordinates as inverse values. The B_{max/inh} value represents the maximal peptide-mediated percentage-inhibition of PHEX-MEPE binding relative to the “non-peptide” control (0% inhibition of binding). This was calculated using a constant analyte flow-solution of 250 nM secPHEX against immobilized MEPE ligand at 6000 RU.

FIG. 35 shows epi-uv fluorescence-imaging of undecalcified tissues; A. calvariae and, B. tibia-femurs, from mice injected with vehicle (HEPES buffer), etidronate (10 mg/kg/day) and PO₄-ASARM-peptide (2 mg/kg/day) as indicated. Injections were given once a day for 12 days (except days 6 and 7) and all animals were injected with calcein (20 mg/kg/day) on days 3, 5, 9 and 11 respectively. Samples were imaged simultaneously on a large Bio-Rad imaging platen after epi uv-illumination-induced fluorescence and digital image-capture (FluorImger^{max} imaging-system). Image saturation was avoided by software-monitoring (Quantity-1 software from Bio-Rad, Hercules, Calif.) and the captured calcein-fluorescent images quantitated by use of the same software (see FIGS. 36a and 36b).

FIG. 36 shows quantitation of whole-fluorescent imaging as depicted in FIG. 35 by use of Bio-Rad (Hercules, Calif.) Quantity 1 software. Both the PO₄-ASARM-peptide and etidronate groups had significant and markedly quenched fluorescence with fluorescent intensity units of 568.7 (SEM=80.2; N=5; P<0.01) and 417.7 (SEM=48.3; N=5; P<0.001) respectively, compared to the vehicle group at 1100.5 (SEM=132.8; N=5). This represents a percentage quenching of 48.4% (P<0.01) for etidronate and 62% (P<0.001) for the PO₄-ASARM-peptide respectively. Graph B illustrates a similar quenching with tibiae-femurs and PO₄-ASARM-peptide group fluorescent intensity of 148.9 (SEM=15.1; N=5; P<0.01) relative to the vehicle at 268.52 (SEM=26.8; N=5). This corresponds to a PO₄-ASARM-peptide mediated fluorescence-signal reduction of 44.5% (P<0.01).

FIG. 37 shows representative photomicrographs of plastic-embedded, undecalcified mice-calvariae from FIGS. 35 and 36. Slides were analyzed under fluorescent microscope at 200× and the calcein-fluorescence captured by digital camera. The control group (vehicle) shows four lamellar fluorescent layers corresponding to the four calcein injections (20 mg/kg/day) given on days 3, 5, 9 and 11 respectively.

FIG. 38 shows corresponding Sanderson staining of representative mice-calvariae, undecalcified cross-sections (×200), embedded in plastic as described for FIG. 37. The darker staining osteoid is clearly visible in grayscale on the upper surface of each sample (A, B and C) and is highlighted with arrows in C. The inset histogram graphically illustrates the marked increase in thickness and thus impaired mineralization in groups B (etidronate) and C (PO₄-ASARM-peptide), with relative thicknesses of 5.4 (SEM=0.29; N=3; P<0.05) and 8.0 (SEM=0.57; N=3; P<0.001) respectively compared to vehicle (A) at 2.3 (SEM=0.33; N=3). Measurements were made from the exact same area (suture) in all three groups.

FIG. 39 shows competitive ELISA plots of percentage-quenching (y-axis) against non-biotinylated peptide (x-axis). Anti-ASARM-peptide polyclonal was first bound onto protein-G 96-micro-well plates. A constant concentration of biotinylated-ASARM-peptide (0.5 ng/mL) was then separately mixed with different concentrations of non-biotinylated ASARM-peptide and then added to the plates. The relative degree of chemiluminescent quenching was then assayed after addition of streptavidin-horseradish-peroxidase conjugate and measurement of light emission by a Bio-Rad FlourImager^{max} system as described in Example VII. Graph A illustrates percentage-quenching data directly plotted against ASARM-peptide (ng/mL) (K_{D} of 7.5 ng/mL and a Qmax (quench maximum) of 98.7%). Graph B illustrates the same data but with a Log₁₀ ASARM-peptide (ng/mL) transform on the X-axis.

FIG. 40 shows graphs of percentage quenching-data plotted against Log₁₀ (serum dilutions ×106). Graph A shows the human sera results (HYP patients (N=9) and normal subjects (N=9)). Graph B shows the results for male mice sera (male hyp-mice (N=3) and male normal siblings (N=3)). In both humans and mice the affected or HYP human/mice plots show a major and parallel shift relative to normals. This indicates an increase in ASARM-peptide epitope in HYP patients and hyp mice. The shift between normal and HYP human-plots is five fold. The shift is more pronounced in mice (approximately six fold). The slopes of both humans and mice are almost identical and parallel. Thus a major increase (five to six-fold) in ASARM-epitopes is present in HYP-human and hyp-mice sera relative to normals.

FIG. 41 is a histogram plot that compares levels of ASARM-epitope(s) in normal human and mice sera relative to HYP/hyp counterparts. Concentrations of ASARM-peptide were calculated from the concentrations of peptide determined at three different dilutions of sera for each individual (see FIG. 40). A five-fold (P=0.007) increase in ASARM-epitopes were measured in HYP-patients (N=9) relative to normal subjects (N=9). A six-fold increase (P=0.008) was measured in male hyp-mice (N=3) compared to normal male siblings (N=3).

FIG. 42 illustrates an immunohistochemical screen of hyp-mouse and normal-mouse renal cross-sections with anti-ASARM-peptide polyclonals. The expression of ASARM-peptide epitopes was dramatically increased in male hyp-mice (upper two panels) compared to normal male siblings mice at 8 weeks (lower two panels). The ASARM-peptide staining is anatomically consistent with the renal proximal-convoluted-tubules. This region also stains positive for the sodium phosphate cotransporter type IIa (NaPi-2a). Nuclear-counterstaining was performed using standard hematoxylin and renal glomeruli are clearly visible in normal and hyp kidneys. Magnification was ×10.

FIG. 43 shows the sequence alignment of human, macaque monkey, murine, and rat MEPE. The consensus sequence is also shown. The NEIP motif 1 and NEIP motif 2 are indicated with bracketed arrows.

### DETAILED DESCRIPTION

MEPE is a secreted RGD-matrix phosphoglycoprotein that promotes renal phosphate excretion and inhibits bone mineralization. MEPE is exclusively expressed in osteoblasts, osteocytes, and odontoblasts under normal conditions. Moreover, MEPE is markedly up-regulated in osteoblasts and bone from mice with hypophosphatemic rickets (HYP) and in oncogenic hypophosphatemic osteomalacia (OHO) tumors.

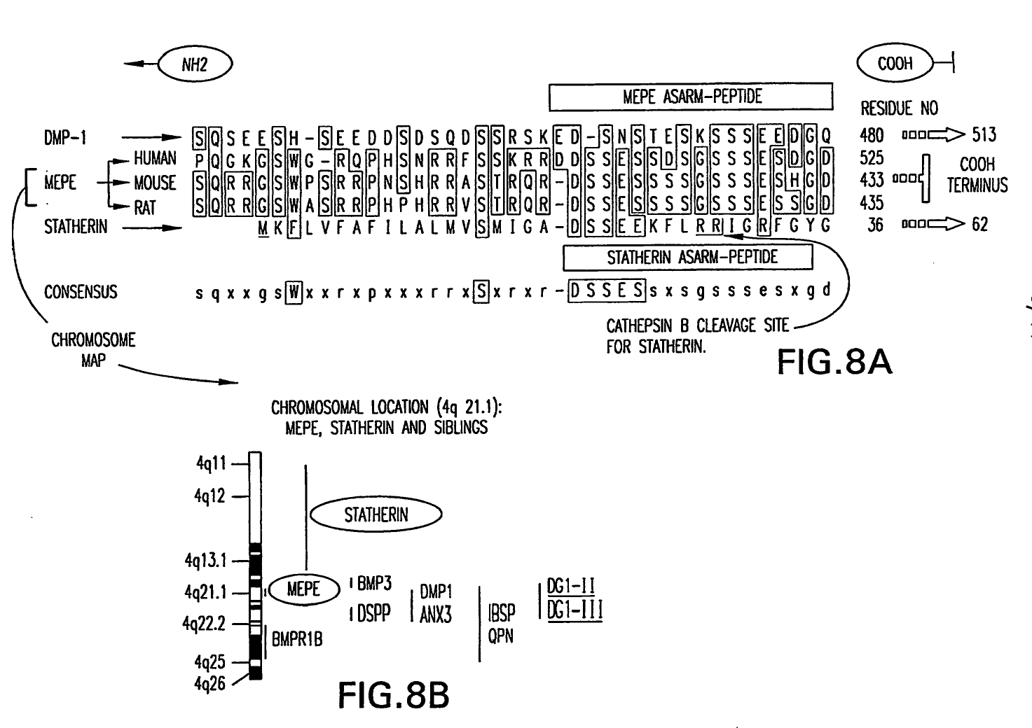

MEPE belongs to the short integrin-binding ligand interacting glycoprotein (SIBLING) family, all of whose members map to chromosome 4q21.1. In addition to MEPE, these proteins include dentin sialophosphoprotein (DSPP), osteopontin (SPP1), dentin-matrix-protein-1 (DMP-1) and bone sialophosphoprotein (BSP); see FIG. 8b.

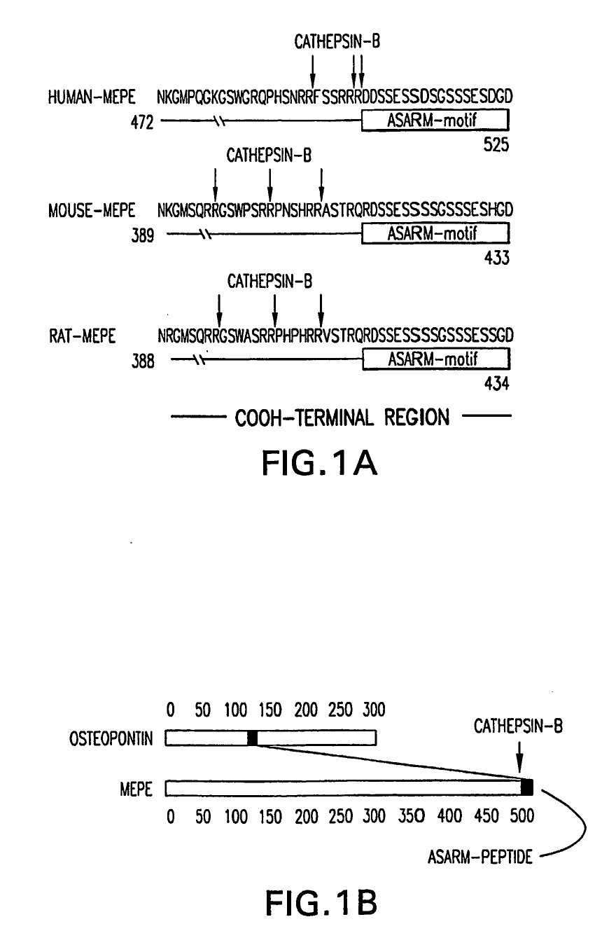

The disclosed subject matter is based on the discovery, as described herein, that a small (2 kDa) carboxy-terminal peptide of MEPE can be used, under various conditions, to either promote or inhibit renal and intestinal phosphate absorption, to promote bone and tooth mineralization and/or inhibit demineralization, and to inhibit soft tissue mineralization. Moreover, this peptide (ASARM-peptide), by virtue of its ability to bind to bone/hydroxyapaptite and physiochemical similarities to bisphosphonates can be a potent inhibitor of bone resorption (a feature of HYP in which ASARM-peptides are elevated in serum) and bone-cancer metastasis. Accordingly, as set forth herein, this small peptide can be used to treat diseases or conditions involving hyperphosphatemia or hypophosphatemia, Pagets disease and cancer (prevention of tumor metastasis). In particular, cancers most likely to spread to bones, breast, prostate, lung, kidney, and thyroid, and multiple myeloma would be particularly amenable to MEPE carboxy-terminal ASARM-peptide therapy. In addition, the MEPE carboxy-terminal peptide can be used to treat diseases involving aberrant bone mineralization (e.g., osteoporosis) or aberrant soft tissue mineralization (such as that resulting from renal disease). The MEPE carboxy-terminal peptide, which is released from MEPE by cleavage by cathepsin-B and/or other proteases, contains an acidic-serine-aspartate-rich motif (ASARM-peptide; see FIG. 1) that is responsible for its activity in vivo.

Also the disclosed subject matter is based on the surprising discovery, as described herein, that MEPE polypeptide and fragments thereof, including ASARM peptides, inhibit tumor growth and restricts tumor size. Accordingly, MEPE, MEPE peptides, and/or ASARM-peptides can be administered to cancer patients to slow the growth of their tumors. Such therapies can be administered alone or as an adjunct therapy to other types of cancer therapies (e.g., chemotherapy, radiation therapy, or surgery). Moreover, combinations of ASARM-peptide and MEPE therapy can be administered to inhibit tumor growth and prevent tumor metastasis to bone and/or soft tissue.

Normal Serum Phosphorus Levels

Normal serum phosphorus concentrations in humans are approximately 3 to 4.5 mg/dL.

Hyperphosphatemia

Hyperphosphatemia in humans is defined by the skilled artisan as serum phosphorus above about 4-5 mg/dL. Hyperphosphatemia is caused by accumulation of phosphate resulting from impaired excretion of phosphate.

For example, as is well known in the art, patients with renal disease can display hyperphosphatemia or hypophosphatemia. Dialysis and restriction of dietary phosphate are insufficient to prevent hyperphosphatemia in patients with renal insufficiency. Complications that arise from hyperphosphatemia include: ectopic calcification of soft tissues (in particular, coronary and aortic calcification is a major problem in hemodialysis patients); secondary hyperparathyroidism, which results in an abnormally high bone turnover due to an increase in osteoblasts and osteoclasts; and renal osteodystrophy. The skilled artisan will understand that patients having hyperphosphatemia will benefit from treatment by administration of ASARM peptide using a dosing regimen that inhibits phosphate resorption in the kidney and/or absorption in the gut (see, e.g., Examples II and III). Under a dosing regimen such that the renal and/or intestinal sodium-phosphate antiporter is continuously saturated with ASARM peptide, the antiporter is inactivated and thus cannot function to effect phosphate absorption/resorption. One of ordinary skill in the art will readily be able to determine the proper dosing regimen to treat such hyperphosphatemic individuals, such that their serum phosphate levels decrease and, ideally, are restored to normal phosphate levels.

The skilled artisan will understand that patients with hyperphosphatemia resulting from a broad variety of diseases and conditions (see, e.g., Table 25-9 (Causes of Hyperphosphatemia) R. Agarwal and J. Knochel, Chapter 25 “Hypophosphatemia and Hyperphosphatemia,” In: Brenner & Rector's The Kidney, 6^{th} ed., 2000, WB Saunders Co., which is incorporated by reference herein for its teachings of diseases and conditions relating to hyperphosphatemia) can be treated using the methods disclosed herein.

Hypophosphatemia

Hypophosphatemia in humans is defined by the skilled artisan as a serum inorganic phosphorus concentration of less than about 2.5 mg/dL (0.83 mM); severe hypophosphatemia is defined as serum phosphorus of about 1.5 mg/dL or less. Hypophosphatemia can be due to inadequate dietary intake, impaired gastrointestinal absorption, or increased renal excretion of phosphorus.

For example, as is well known to the skilled artisan, hypophosphatemia can result from severe diarrhea, starvation, Cushing syndrome, intestinal malabsorption syndromes, kidney transplantation, acute renal failure, end-stage renal disease, chronic alcoholism and alcohol withdrawal, severe thermal bums, acute malaria, and various drug therapies. See Table 25-5 (Causes of Severe Hypophosphatemia), R. Agarwal and J. Knochel, Chapter 25 “Hypophosphatemia and Hyperphosphatemia,” In: Brenner & Rector's The Kidney, 6^{th} ed., 2000, WB Saunders Co, which has been incorporated herein by a previous reference.

Parameters for identifying patients who require treatment for hypophosphatemia are well known in the art. For example, if the hypophosphatemia occurs in patients who are suspected to have previous renal and gastrointestinal phosphorus losses, or who have evidence of alcoholism, malnutrition, or increased catabolism, and/or if there is evidence of a phosphorus depletion syndrome, such patients should be treated for their hypophosphatemic conditions. See, e.g., “Treatment of Hypophosphatemia,” R. Agarwal and J. Knochel, supra. The skilled artisan will understand that such patients can be treated using the methods disclosed herein, by administration of ASARM peptide using a dosing regimen that enhances phosphate absorption/resorption (see, e.g., Example II). Administration of a single dosage of ASARM peptide that is sufficient to inhibit the activity of the sodium/phosphate antiporter will result in an increase of sodium-phosphate antiporter molecules in the kidney and/or intestine, via transcriptional or post-transcriptional up-regulation. Accordingly, phosphate absorption/resorption will be temporarily inhibited; however, once ASARM peptide is cleared from the body, there will be a net increase in phosphate absorption/resorption, due to an increase in the number of sodium-phosphate antiporter molecules. This process can be repeated as deemed necessary by the skilled artisan. One of ordinary skill in the art will readily be able to determine the proper dosing regimen to treat such hypophosphatemic individuals, such that their serum phosphate levels rise and, ideally, are restored to normal phosphate levels.

Osteoporosis

Bone, which is the body's largest reservoir of phosphorus, is an extremely metabolically active tissue that undergoes constant remodeling. In normal bone, new bone production by osteoblasts and bone resorption by osteoclasts is in net equilibrium. However, in patients with osteoporosis, there is a net loss of bone. Pharmaceutical compounds such as the bisphosphonates, which inhibit bone resorption by inhibiting osteoclast activity, are used to treat patients with osteoporosis. In accordance with the methods disclosed herein, ASARM peptide can be administered to such patients as a “biological bisphosphonate” to inhibit bone loss. As shown in Example III, ASARM peptide inhibits serum alkaline phosphatase activity, which is a measure of osteoclast activity, and thus, bone loss. Accordingly, ASARM peptide can be administered such that it inhibits osteoclast activity and thus inhibits bone loss, and one of ordinary skill in the art will understand how to determine the proper dosing regimen to treat such patients, such that the results obtained are comparable with or better than those results obtained with bisphosphonate compounds currently in use for such treatments.

Periodontal Disease

Gum disease (e.g., gingivitis, or bleeding gums) due to periodontal infections can lead to ectopic gum mineralization, which inhibits healing of the gums and worsens the periodontal disease. Accordingly, such periodontal disease can be treated or prevented by application of ASARM peptide to the affected area (e.g., as a mouthwash, in a toothpaste, or as some other orally-applied or otherwise-administered preparation), such that ectopic gum mineralization is inhibited and progression of the periodontal disease is inhibited, as will be understood by the skilled artisan.

Diseases Involving Pathologically High Levels of Endogenous ASARM Peptide

MEPE is markedly up-regulated in osteoblasts from mice with X-linked hypophosphatemic rickets (HYP) and in tumors from patients with oncogenic hypophosphatemic osteomalacia (OHO). The primary defect in HYP is due to mutation and functional inactivation of the gene-product PHEX, resulting in defective calcification of bone, renal phosphate wasting (hypophosphatemia) and abnormal vitamin-D metabolism. Prior to the present discovery, the molecular mechanisms responsible for these abnormalities were not known. However extensive studies in HYP mice have indicated that the defect in mineralization and renal function is mediated by circulating factor(s) derived from PHEX-defective osteoblasts. Thus, in HYP, the evidence strongly implicates one or more circulating humoral factors secreted by HYP osteoblasts, which affect mineralization and renal phosphate handling.

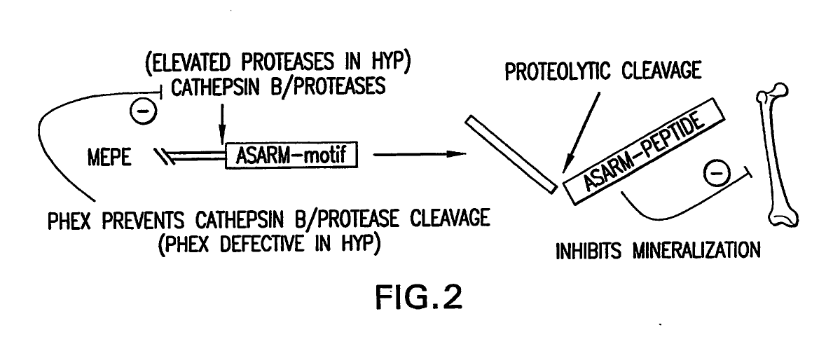

The present inventor has discovered that, under normal conditions, PHEX inhibits production of ASARM peptide in vivo, by binding to and preventing cleavage of MEPE at a carboxy-terminal cathepsin-B cleavage site (see FIGS. 1 and 2). Because PHEX is functionally inactive in HYP patients, there is an abnormally high level of MEPE cleavage. As a result, these patients produce pathologically elevated levels of ASARM peptide, which leads to hypophosphatemia and inhibition of bone mineralization (rickets).

Similarly, since patients with OHO tumors produce abnormally elevated levels of MEPE, such patients also produce pathologically elevated levels of ASARM peptide, which leads to hypophosphatemia and a decrease in bone mineralization (osteomalacia).

Accordingly, as described herein, such diseases associated with abnormally elevated levels of endogenous ASARM peptide can be treated by blocking the biological activity of endogenous ASARM peptide (e.g., with a blocking antibody or other compound, as described herein), in order to decrease overall phosphate excretion, increase phosphate resorption in the kidney and/or increase phosphate absorption in the intestine, and promote bone mineralization and/or inhibit bone demineralization.

Cancer

Tumors that have been found to express full-length MEPE (see, e.g., De Beur “Tumors associated with oncogenic osteomalacia express genes important in bone and mineral metabolism.” J Bone Miner Res 17:1102-10, 2002) tend to grow slowly and to be very small in size. Moreover, as shown in Example IV herein, tumors that secrete MEPE grow more slowly than tumors that do not secrete MEPE. Without being bound by theory, it is hypothesized that the presence of excessive MEPE in the body stimulates the immune system, thereby strengthening the vigilance of the immune system and allowing it to more effectively recognize tumor cells. Accordingly, full-length MEPE can be administered to patients in order to slow or inhibit tumor growth, as can biologically active fragments of MEPE that slow or inhibit tumor growth.

In a second approach, tumor cells can be obtained from patients and engineered to secrete MEPE (e.g., by transfecting the cells ex vivo with an expression vector encoding MEPE), after which the engineered tumor cells are returned to the patient to sensitize the immune system to the tumor. Such methods of tumor “vaccination” are well known in the art (see, e.g., Soiffer, et al., “Vaccination with irradiated, autologous melanoma cells engineered to secrete granulocyte-macrophage colony-stimulating factor by adenoviral-mediated gene transfer augments antitumor immunity in patients with metastatic melanoma,” J Clin Oncol 21(17):3343-50, 2003).

MEPE therapy can also be administered in combination with other types of anti-cancer therapies, e.g., surgical, chemotherapeutic, and/or radiation therapy. Supplemental phosphorus can be administered as necessary to cancer patients treated with MEPE, to ameliorate any hypophosphatemic effects that might occur from MEPE therapy.

ASARM-peptide therapy can also be used to treat diverse bone-cancer metastases. A pernicious feature of many cancers is the propensity of cells to break away from the primary cancerous-tumor. These cells then travel to other areas of the body through either the bloodstream or lymphatic channels and bone is a rich environment for cancer cells to grow and metastasize. Bone metastases are not the same as primary bone cancers as they do not necessarily originate from bone. The spine, ribs, pelvis and bones near the hips and shoulders are most susceptible to metastatic tumor-cells. These tumors are the most frequent causes of pain in people with cancer and cause fractures, hypercalcemia (high blood calcium levels because calcium is released from damaged bones), and other symptoms and complications that result in severe debilitation and morbidity. Breast, prostate, lung, kidney, thyroid and multiple myeloma cancers are most likely to spread to bones. Bisphosphonates share many biological and physiochemical features with the ASARM-peptide and have been used to treat bone cancer metastases. These shared features include low pI, high charge, phosphorylation, small size, protease-resistance, inhibitors of phosphate-transport, vitamin D metabolism, mineralization inhibitor, bind to hydroxyapatite and bone etc.

Not wishing to being bound by theory, it is believed that the ASARM-peptide like bisphosphonates can act by binding to bone and interrupting the “vicious cycle” of bone metastases, which begins when tumor cells settle into bone and stimulate the release of factors which cause the breakdown of bone tissue. The destruction of bone cells releases factors which stimulate the production of more tumor cells. Thus the ASARM-peptide like bisphosphonates can prevent the destruction of bone by cancer metastases and even cause those metastatic tumors to shrink. Unlike bisphosphonates, the ASARM-peptide (a biological-bisphosphonate) will not remain irreversibly bound to bone and will have a much reduced toxicity. Moreover, a combination of full-length MEPE and/or MEPE therapy (tumor growth inhibitor) and ASARM-peptide therapy (anti-metastases and tumor growth) will provide a biologically safe and effective treatment regimen for many cancers.

In this specification and in the claims that follow, reference is made to a number of terms that shall be defined to have the following meanings.

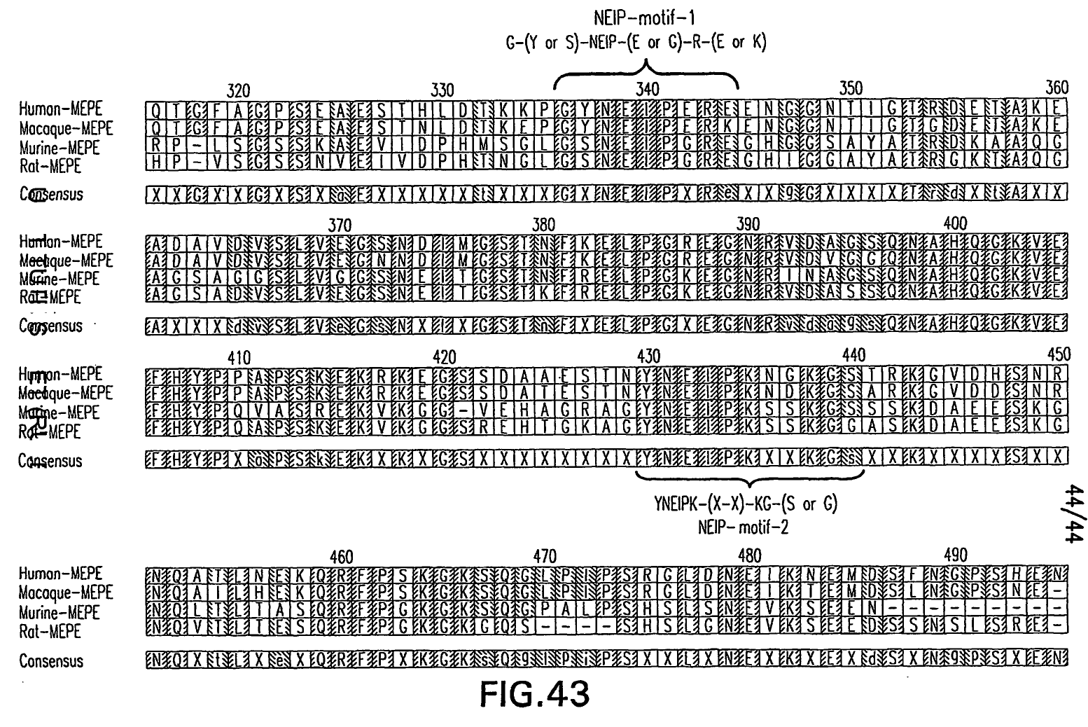

By “MEPE” is meant Matrix Extracellular Phosphoglycoprotein, a polypeptide encoded by a gene that was first cloned from an osteomalacia-causing tumor, as described in Rowe et al. (“MEPE, a New Gene Expressed in Bone Marrow and Tumors Causing Osteomalacia” Genomics 67:54-68, 2000). Human MEPE, amino acid variants thereof, and non-human (e.g., murine) homologs thereof, can be used in the methods disclosed herein (e.g., for treating cancer), as will be understood by those of ordinary skill in the art. “MEPE” includes the full-length man, mouse, rat, and macaque-monkey polypeptides given in SEQ ID NOS. 17, 18, 19, and 20, respectively. “MPEP” and “peptides derived from MEPE” include fragments of the full-length sequences. Examples of such fragments include, but are not limited to, SEQ ID NOS. 21, 22, 23, and 24 and any sequence that contains the NEIP motif 1 and NEIP motif 2 shown in FIG. 43.

By “endogenous ASARM peptide” is meant an ASARM peptide equivalent to the ASARM peptide that is produced in vivo by cleavage of full-length MEPE by cathepsin B (see, e.g., FIG. 1).

By “isolated ASARM peptide” is meant an ASARM peptide that has been obtained, for example, by expression of a recombinant nucleic acid encoding the polypeptide (e.g., in a cell or in a cell-free translation system), by extraction from a natural source (e.g., a prokaryotic or eukaryotic cell), or by chemically synthesizing the peptide. Isolated ASARM peptides disclosed herein have ASARM biological activity as defined herein, and can contain an amino acid sequence selected from one of ASARM peptide sequences shown in FIG. 8a (e.g., from human or non-human MEPE, or from DMP-1 or Statherin). ASARM peptides disclosed herein can also comprise any ASARM peptide fragment or variant thereof (see FIG. 1a and FIG. 8a) having ASARM biological activity, e.g., peptides comprising DSESS (SEQ ID NO. 8) or SSSES (SEQ ID NO. 9) (see FIG. 8a). An isolated ASARM peptide can also be from any human or non-human SIBLING protein, as can be appreciated from FIGS. 8a and 8b. The isolated ASARM peptide can be unphosphorylated or can be phosphorylated at one or more of the serine residues, including phosphorylation at each serine residue present within the peptide. Examples of suitable ASARM-peptide sequences include, but are not limited to, SEQ ID NOS. 1, 2, 3, 4, 5, 8, 9, 13, 14, and 16.

By “biological activity of an ASARM peptide” or “ASARM biological activity” and the like is meant the ability of an ASARM peptide to bind to the renal and/or intestinal sodium/phosphate antiporter and decrease antiporter activity such that renal resorption of phosphate and/or intestinal absorption of phosphate is decreased or even fully inhibited. Therefore, decreasing the biological activity of endogenous ASARM peptide in patients with diseases involving pathologically elevated levels of ASARM peptide will decrease overall phosphate excretion, increase phosphate resorption in the kidney and/or increase phosphate absorption in the intestine, and promote bone mineralization and/or inhibit bone demineralization.

Accordingly, the skilled artisan will understand that a compound that blocks the biological activity of an ASARM peptide can be a compound (e.g., an antibody, peptide, or other compound) that, e.g., binds to and sequesters ASARM peptide, thereby preventing interaction of the ASARM peptide with the renal and/or intestinal sodium/phosphate antiporter(s); or binds to the renal and/or intestinal sodium/phosphate antiporter(s) and blocks the site on the antiporter(s) to which ASARM binds, without blocking the phosphate transport activity of ASARM.

By “abnormal phosphate metabolism” is meant a level of serum phosphate, a level of phosphate absorption, resorption or excretion, and/or a level of bone phosphate-dependent bone formation or resorption that differs from the level(s) found in a normal individual with normal phosphate metabolism and no evidence of a disease involving altered phosphate metabolism. Abnormal levels of phosphate can be measured in blood, serum, urine, saliva, or bone, for example.

By “modulate” is meant to alter, either by increase or decrease.

By “phosphate-dependent disease” or “phosphate-dependent condition” is meant an affliction, disorder, or physiological state that involves or affects phosphate absorption, resorption, excretion, and/or tissue mineralization and/or demineralization or that is exacerbated by abnormal levels of phosphate absorption, resorption, excretion, and/or tissue mineralization and/or demineralization. Examples of phosphate-dependent diseases and conditions include, for example, osteoporosis, bone cancers, periodontal disease, dental caries, dental ectopic mineralization, renal osteodystrophy, renal transplant, end stage renal disease, renal toxicity, renal disease, ectopic renal-calcification, arterial calcification, tumor-induced osteomalacia, hypophosphatemia, linear sebaceous naevua syndrome with hypophosphatemia, neurofribromatosis with hypophosphatemia, and hyperphosphatemia.

By “antagonist” is meant any compound, natural or synthetic, that inhibits the activity of ASARM, MEPE, or ASARM or MEPE agonists.

By “sample” is meant an animal (i.e., vertebrate) including a cold-blooded animal (e.g., fish, reptile, or amphibian), a warm-blooded animal (e.g., a human, a farm animal, a domestic animal, or a laboratory animal, as described herein below); any body fluid (e.g., but not limited to, blood, urine, cerebrospinal fluid, semen, sputum, saliva, tears, joint fluids, body cavity fluids, or washings), tissue, or organ obtained from an animal; a cell (either within an animal, taken directly from an animal, or a cell maintained in culture or from a cultured cell line); a lysate (or lysate fraction) or extract derived from a cell; a molecule derived from a cell or cellular material (e.g., but not limited to, a MEPE molecule, an ASARM peptide molecule, or a sodium-phosphate antiporter molecule used in a screening assay); or a synthetic or naturally-occurring compound (e.g., a test compound), which is assayed or analyzed for its ability to modulate phosphate metabolism activity according to the methods disclosed herein.

By “subject” is meant any animal to which the ASARM peptide or MEPE, or modulator thereof, or a biologically active fragment thereof, disclosed herein is administered for therapeutic or experimental purposes, or to regulate phosphate metabolism. For example, the subject can be a cold-blooded animal, such as a fish, a reptile, or an amphibian, or the subject can be a warm-blooded animal, such as a human, a farm animal, a domestic animal, or a laboratory animal, as described herein. The subject can also be a cell or a DNA molecule in a cell-free environment (for example, a DNA molecule used as a transcription template in a cell-free transcription assay).

By “inhibition of tumor growth” is meant to reduce, decrease, or inhibit cellular proliferation or overall tumor size. Also meant by “inhibit tumor growth” is to inhibit angiogenesis or metastases. Preferably, the decrease (level of inhibition) is by at least about 20%, and can be larger, e.g., by at least about 21% to 40%, 41% to 60%, 61% to 80%, 81% to 90%, or 91% to 100%.

By prevention and/or inhibition of tumor metastasis is meant the reduction or complete inhibition of the spread of cancer cells. Preferably, the decrease (level of inhibition) is by at least about 20%, and can be larger, e.g., by at least about 21% to 40%, 41% to 60%, 61% to 80%, 81% to 90%, or 91% to 100%. Cancer cells can break away from a primary tumor and travel through the bloodstream or lymphatic system to other parts of the body. Cancers are capable of spreading through the body by two mechanisms: invasion and metastasis. Invasion is the direct migration and penetration by cancer cells into neighboring tissues. Metastasis is the ability of cancer cells to penetrate into lymphatic and blood vessels, circulate through the bloodstream, and then grow in a new focus (metastasize) in normal tissues elsewhere in the body.

By “reaction mixture” is meant any environment in which phosphate is regulated, transported, released, or absorbed, and can be used to measure phosphate metabolism activity of a test substance or phosphate metabolism activity within a sample. The reaction mixture can be, for example, within a test tube or within a well of a tissue culture dish or microtiter plate containing phosphate either in soluble form or bound to a solid phase; upon the surface of a filter or a polymer bead carrying phosphate; or within an animal, animal tissue, cell, cell lysate, or cell extract containing phosphate.

By “an effective amount” of an ASARM peptide or MEPE is meant an amount that is useful for performing the stated function (e.g., treatment or prevention of a disease or condition involving abnormal phosphate metabolism, or treatment or prevention of cancer) of the compound for which an effective amount is expressed. As will be described below, the exact amount required will vary, depending upon recognized variables, such as the subject to be treated, the reason for treatment, and the specific MEPE or ASARM-containing compound employed. Thus, it is not possible to specify an exact “effective amount.” However, as described below, an appropriate effective amount can be determined by one of ordinary skill in the art using only routine experimentation.

By “in need of” is meant a subject, such as a human, a farm animal, a domestic animal, or a laboratory animal, as described herein, or a cell, a cell-free transcription system, or a DNA molecule, for which it is desirable to treat, prevent, inhibit, or regulate a phosphate-dependent disease or condition, or that is subjected to experimental administration of peptide-containing compounds disclosed herein in order to study their pharmacological properties, such as, but not limited to, safety, efficacy, or physiological effects.

By “treat” is meant to administer a compound disclosed herein (e.g., an ASARM peptide, MEPE, or a mimetic or antagonist thereof) to a subject or a sample in order to: eliminate a phosphate-dependent disease or condition within a subject or sample; stabilize or delay the progression of a phosphate-dependent disease or condition within a subject or sample; or decrease the frequency or severity of symptoms and/or recurrences of a phosphate-dependent disease or condition within a subject or sample.

By “prevent” is meant to minimize the chance that a subject will develop a phosphate-dependent disease or condition, or to delay the development of a phosphate-dependent disease or condition in a subject. For example, the ASARM peptides disclosed herein can be administered to minimize the chance that a subject will contract osteoporosis. For subjects belonging to families having hereditary phosphate-dependent diseases and conditions, such as phosphate-dependent hypophosphatemia, phosphate-dependent hyperphosphatemia, osteoporosis, periodontal disease, dental caries, renal or arterial calcification, or bone cancer, for example, ASARM peptide therapy can be initiated prior to disease onset, thereby lessening the chance that the subject will fall prey to the disease, and/or delaying the onset of the disease, relative to the time that onset would have occurred, had ASARM peptide therapy not been initiated.

By “about” is meant a 10% of a recited value. Also, ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The terms “high,” “elevate,” or “enhance” refer to increases above basal levels, e.g., as compared to a control. The terms “low,” “reduce,” or “inhibit” refer to decreases below basal levels, e.g., as compared to a control. By “control” is meant either a subject, organ, tissue, or cell lacking a disease or injury, or a subject, organ, tissue, or cell in the absence of a particular variable such as a therapeutic agent. A subject, organ, tissue, or cell in the absence of a therapeutic agent can be the same subject, organ, tissue, or cell before or after treatment with a therapeutic agent or can be a different subject, organ, tissue, or cell in the absence of the therapeutic agent. Comparison to a control can include a comparison to a known control level or value known in the art. Thus, basal levels are normal in vivo levels prior to, or in the absence of, the addition of an agent (e.g., a therapeutic agent) or another molecule.

There are a variety of compositions disclosed herein that are amino acid based, including, for example, MEPE and ASARM. Thus, as used herein, “amino acid,” means the typically encountered twenty amino acids which make up polypeptides. In addition, it further includes less typical constituents which are both naturally occurring, such as, but not limited to formylmethionine and selenocysteine, analogs of typically found amino acids, and mimetics of amino acids or amino acid functionalities. Non-limiting examples of these and other molecules are discussed herein.

As used herein, the terms “peptide,” “polypeptide,” and “protein” refer to a class of compounds composed of amino acids chemically bound together. Non-limiting examples of these and other molecules are discussed herein. In general, the amino acids are chemically bound together via amide linkages (CONH); however, the amino acids may be bound together by other chemical bonds known in the art. For example, the amino acids may be bound by amine linkages. Peptide as used herein includes oligomers of amino acids and small and large peptides, including polypeptides and proteins.

In addition to the disclosed peptides, polypeptides, and proteins contemplated herein are derivatives and variants of the disclosed peptides, polypeptides, and proteins that also function in the disclosed methods and compositions. Protein variants and derivatives are well understood to those of skill in the art and in can involve amino acid sequence modifications. For example, amino acid sequence modifications typically fall into one or more of three classes: substitutional, insertional, and deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions, or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Table 1 and are referred to as conservative substitutions.

TABLE 1
Amino Acid Substitutions
Original Residue Exemplary Conservative Substitutions, others are
known in the art.

Ala; Ser
Arg; Lys, Gln
Asn; Gln, His
Asp; Glu
Cys; Ser
Gln; Asn, Lys
Glu; Asp
Gly; Pro
His; Asn, Gln
Ile; Leu, Val
Leu; Ile, Val
Lys; Arg, Gln;
Met; Leu, Ile
Phe; Met, Leu, Tyr
Ser; Thr
Thr; Ser
Trp; Tyr
Tyr; Trp, Phe
Val; Ile, Leu

The substitutions which in general are expected to produce the greatest changes in the protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g., Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T E Creighton, Proteins: Structure and Molecular Properties, WH Freeman & Co., San Francisco, pp. 79-86, 1983), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NOS. 1-5 set forth a particular sequence of ASARM. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman (Adv Appl Math 2:482, 1981), by the homology alignment algorithm of Needleman and Wunsch (J Mol Biol 48:443, 1970), by the search for similarity method of Pearson and Lipman (Proc Natl Acad Sci USA 85:2444, 1988), by computerized implementations of these algorithms (GAP, BESTFIT, FASTA, and TFASTA in the Wisconsin Genetics Software Package, Genetics Computer Group, Madison, Wis.), or by inspection.

The same types of homology can be obtained for nucleic acids by for example the algorithms disclosed in Zuker (Science 244:48-52, 1989), Jaeger, et al., (Proc Natl Acad Sci USA 86:7706-7710, 1989), and Jaeger, et al., (Methods Enzymol 183:281-306, 1989) which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

The peptides, polypeptides, and polypeptide fragments disclosed herein can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the sodium channels disclosed herein, for example, can be synthesized by standard chemical reactions. For example, a peptide or polypeptide fragment can be synthesized and not cleaved from its synthesis resin whereas another peptide or polypeptide fragment can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an sodium channel, or fragment thereof. (See Grant G A (1992) Synthetic Peptides: A User Guide. W. H. Freeman and Co., New York, N.Y. (1992); Bodansky M and Trost B., Ed. Principles of Peptide Synthesis. Springer-Verlag Inc., New York, N.Y. (1993)). Alternatively, the peptide or polypeptide is independently synthesized in vivo as described above.

For example, enzymatic ligation of cloned or synthetic peptide segments allow relatively short peptide fragments to be joined to produce larger peptide fragments, polypeptides or whole protein domains (Abrahmsen, et al. (1991) Biochemistry 30:4151). Alternatively, native chemical ligation of synthetic peptides can be utilized to synthetically construct large peptides or polypeptides from shorter peptide fragments. This method consists of a two step chemical reaction (Dawson, et al., Science 266:776-779, 1994). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini, et al., FEBS Lett 307:97-101, 1992; Clark-Lewis, et al., J Biol Chem 269:16075, 1994; Clark-Lewis, et al., Biochemistry 30:3128, 1991; Rajarathnam, et al., Biochemistry 33:6623-30, 1994).

Alternatively, unprotected peptide segments are chemically linked where the bond formed between the peptide segments as a result of the chemical ligation is an unnatural (non-peptide) bond (Schnolzer, et al., Science, 256:221, 1992). This technique has been used to synthesize analogs of protein domains as well as large amounts of relatively pure proteins with fall biological activity (deLisle Milton, et al. Techniques in Protein Chemistry IV. Academic Press, New York, pp. 257-267 (1992)).

As this specification discusses various amino acid sequences it is understood that the nucleic acids that can encode those amino acid sequences are also disclosed. This would include all degenerate sequences related to a specific amino acid sequence, i.e., all nucleic acids having a sequence that encodes one particular amino acid sequence as well as all nucleic acids, including degenerate nucleic acids, encoding the disclosed variants and derivatives of the amino acid sequences. Thus, while each particular nucleic acid sequence may not be written out herein, it is understood that each and every sequence is in fact disclosed and described herein through the disclosed amino acid sequence.

There are a variety of compositions disclosed herein that are nucleic acid based, including for example the nucleic acids that encode, for example, MEPE. Thus, as used herein, “nucleic acid” means a molecule made up of, for example, nucleotides, nucleotide analogs, or nucleotide substitutes. Non-limiting examples of these and other molecules are discussed herein. A nucleic acid can be double stranded or single stranded. It is understood that, for example, when a vector is expressed in a cell the expressed mRNA will typically be made up of A, C, G, and U.

As used herein, “nucleotide” is a molecule that contains a base moiety, a sugar moiety and a phosphate moiety. Nucleotides can be linked together through their phosphate moieties and sugar moieties creating an internucleoside linkage. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

“Nucleotide analog,” as used herein, is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

“Nucleotide substitutes,” as used herein, are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger, et al., Proc Natl Acad Sci USA, 86:6553-6556, 1989).

A “Watson-Crick interaction” is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A “Hoogsteen interaction” is the interaction that takes place on the Hoogsteen face of a nucleotide or nucleotide analog, which is exposed in the major groove of duplex DNA. The Hoogsteen face includes the N7 position and reactive groups (NH₂ or O) at the C6 position of purine nucleotides.

“Deletion,” as used herein, refers to a change in an amino acid or nucleotide sequence in which one or more amino acid or nucleotide residues, respectively, are absent relative to the reference sequence.

“Insertion” or “addition,” as used herein, refers to a change in an amino acid or nucleotide sequence resulting in the addition of one or more amino acid or nucleotide residues, respectively, as compared to the reference sequence.

“Substitution,” as used herein, refers to the replacement of one or more amino acids or nucleotides by one or more different amino acids or nucleotides, respectively, in a reference sequence.

“Isolated,” as used herein refers to material, such as a nucleic acid or a polypeptide, which is: (1) substantially or essentially free from components which normally accompany or interact with it as found in its naturally occurring environment. Although, the isolated material optionally comprises material not found with the material in its natural environment; or (2) if the material is in its natural environment, the material has been synthetically (non-naturally) altered by deliberate human intervention to a composition and/or placed at a locus in the cell (e.g., genome or subcellular organelle) not native to a material found in that environment. The alteration to yield the synthetic material can be performed on the material within or removed from its natural state.

Therapeutic uses of ASARM and MEPE

Phosphate and other minerals play a major role in cellular function, as well as in other extra-cellular physiological processes. It is desirable to employ ASARM therapy or anti-ASARM therapy, when possible, for the treatment, prevention, or regulation of a broad array of diseases and conditions involving altered phosphate metabolism. Compounds having phosphate regulatory activity, such as the ASARM peptide and MEPE polypeptide, are characterized by the ability to regulate levels of serum phosphate, as well as levels of phosphate in bones and teeth, for example. As such, they can be used in any instance in which it can be desirable to modulate phosphate activity.

For example, the ASARM peptides disclosed herein, or inhibitors thereof, can be used to regulate hypophosphatemia. Hypophosphatemia is defined as a decrease in the level of serum phosphate of a subject. The ASARM peptide, or inhibitors thereof, can therefore be used to treat, control, or ameliorate the symptoms of hereditary hypophosphatemic rickets with hypercalcuria, autosomal dominant hypophosphatemic rickets, receptor-defective rickets, familial rickets, vitamin-D dependent rickets type-1, defective 25-hydroxylase, Fanconi syndrome, oncogenous syndrome, osteodystrophy, or metaphyseal dystrophy, for example.

The ASARM peptides disclosed herein can also be used to regulate hyperphosphatemia. Hyperphosphatemia occurs when phosphorus load (from GI absorption, exogenous administration, or cellular release) exceeds renal excretion and uptake by bones, teeth, and other tissues. The ASARM peptides can therefore be used to treat, control, or ameliorate the symptoms of end stage renal disease, renal osteodystrophy, chronic renal disease, renal toxicity, calcification of kidneys, or calcification of arteries, for example.

The ASARM peptides and inhibitors thereof can also be used to regulate bone demineralization, osteoporosis, periodontal disease, or dental caries.

The ASARM peptides and/or other MEPE derived peptides and/or MEPE can also be used to inhibit tumor growth and/or bone and/or soft-tissue cancer metastasis associated with all tissues, cells, and types in general, including for example, breast, prostate, lung, kidney, thyroid, multiple myeloma cancers, fibrous dysplasia of bone, haemangiopericytomas, gynaecologic cancers, bladder cancer, laryngeal cancer and gastrointestinal cancers mesenchymal tumors, hemangiopericytomas, neurofibromas, tumors associated with linear sebaceous nevus syndrome, oat cell carcinomas, sarcomas, lymphomas (Hodgkin's and non-Hodgkin's), leukemias, carcinomas, carcinomas of solid tissues, squamous cell carcinomas, adenocarcinomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

Full-length MEPE and/or ASARM-peptide and/or MEPE peptides can also be used to treat various types of cancer. Tumors that have been found to express MEPE (see, e.g., De Beur, “Tumors associated with oncogenic osteomalacia express genes important in bone and mineral metabolism,” J Bone Miner Res 17:1102-1110, 2002) tend to grow slowly and to be very small in size. Moreover, as shown in Example IV herein, MEPE inhibits tumor growth. Therefore, MEPE can be administered to patients in order to slow or inhibit tumor growth. MEPE therapy can also be administered in combination with other types of anti-cancer therapies, e.g., surgical, chemotherapeutic, and/or radiation therapy.

Methods of Administration

The ASARM peptides and inhibitors thereof, MEPE polypeptides, and compounds identified using any of the methods disclosed herein can be administered to subjects with a pharmaceutically acceptable diluent, carrier, or excipient, in unit dosage form. By “pharmaceutically acceptable” is meant a material that is not biologically or otherwise undesirable, i.e., the material can be administered to an individual along with an ASARM peptide, inhibitor thereof, or MEPE polypeptide, without causing any undesirable biological effects or interacting in a deleterious manner with any of the components of the pharmaceutical composition in which it is contained. Conventional pharmaceutical practice can be employed to provide suitable formulations or compositions to administer such compositions to subjects. Any appropriate route of administration can be employed, for example, but not limited to, intravenous, parenteral, transcutaneous, subcutaneous, intramuscular, intracranial, intraorbital, ophthalmic, intraventricular, intracapsular, intraspinal, intracisternal, intraperitoneal, intranasal, intrarectal, intravaginal, aerosol, or oral administration. Therapeutic formulations can be in the form of liquid solutions or suspensions; for oral administration, formulations can be in the form of tablets or capsules; for intranasal formulations, in the form of powders, nasal drops, or aerosols; for intravaginal formulations, vaginal creams, suppositories, or pessaries; for transdermal formulations, in the form of creams or distributed onto patches to be applied to the skin; for dental formulations, in the form of mouthwashes or toothpastes.

Methods well known in the art for making formulations are found in, for example, Remington: The Science and Practice of Pharmacy (19th ed.) ed. A R Gennaro, Mack Publishing Company, Easton, Pa. 1995. Formulations for parenteral administration can, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers can be used to control the release of the compounds. Other potentially useful parenteral delivery systems for molecules disclosed herein include ethylene-vinyl acetate copolymer particles, osmotic pumps, implantable infusion systems, and liposomes. Formulations for inhalation can contain excipients, for example, lactose, or can be aqueous solutions containing, for example, polyoxyethylene-9-lauryl ether, glycocholate and deoxycholate, or can be oily solutions for administration in the form of nasal drops, or as a gel.

Dosage

The ASARM peptides, ASARM inhibitors, and MEPE polypeptides disclosed herein can be administered to a subject in an amount sufficient to modulate phosphate metabolism in a subject in need thereof, or to treat, prevent, inhibit, or regulate a phosphate-dependent condition in a subject in need of such treatment, prevention, inhibition, or regulation. One of ordinary skill in the art will understand that optimal dosages used will vary according to the individual being treated, the particular compound being used, and the chosen route of administration. The optimal dosage will also vary among individuals on the basis of age, size, weight, gender, and physical condition. Methods for determining optimum dosages are described, for example, in Remington: The Science and Practice of Pharmacy (19th ed.) ed. A R Gennaro, Mack Publishing Company, Easton, Pa. 1995.

The amount of active ingredient that can be combined with a carrier material to produce a single dosage form will vary depending upon the disease treated, the animal species, and the particular mode of administration. A therapeutically effective amount can be determined by routine experimentation and by analogy from the amounts used to treat the same disease states with analogous phosphate-regulatory compounds, such as the bisphosphonates (which can be used to treat osteoporosis and osteomalacia, and which can promote either phosphate retention or excretion, depending upon the dosage regimen administered, as is well known in the art), and sevelamer hydrochloride (RENAGEL, Genzyme, Cambridge, Mass.), which is used to prevent ectopic calcification and renal osteodystrophy in patients with renal failure. In general, about 50 μg to about 500 mg of ASARM peptide per day is administered. For example, for treatment of humans, an ASARM peptide disclosed herein could be administered in an amount ranging from approximately 0.001 to 10 mg/kg of body weight. The compounds can be administered daily (e.g., one to four times per day) or can be administered weekly, monthly, or sporadically, as is well known in the art.

An effective dose of an ASARM peptide, an ASARM inhibitor, or a MEPE polypeptide can be delivered by pulsatile infusion, continuous infusion, bolus administration, or a combination of all three. The volume of the bolus (or total volume of multiple boluses in the case of a “pulsed” delivery) is selected accordingly to deliver an effective amount of the composition within the ranges prescribed above.

Efficacy

The efficacy of administration of a particular dose of an ASARM peptide, ASARM inhibitor, or MEPE polypeptide can be determined by evaluating the particular aspects of the medical history, signs, symptoms, and objective laboratory tests that are known to be useful in evaluating the status of a subject requiring treatment, prevention, inhibition, or regulation of a phosphate-dependent disease or condition. These signs, symptoms, and objective laboratory tests will vary, depending upon the particular disease or condition being treated or prevented, as will be known to any clinician who treats such subjects or to a researcher conducting experimentation in this field. For example, if, based on a comparison with an appropriate control group and knowledge of the normal progression of disease in the general population or the particular individual: 1) a subject's frequency or severity of recurrences is shown to be improved, 2) the progression of the disease is shown to be stabilized or delayed, or 3) the need for use of other medications for treating the condition or disease is lessened or obviated, then a particular treatment will be considered efficacious.

In a specific example, in using the MEPE polypeptide disclosed herein to treat a tumor, a slowing or partial or complete inhibition of tumor growth or metastases, or even shrinkage of the tumor, are indications that the MEPE polypeptide treatment is efficacious. Similarly, a decrease in the signs or symptoms of a diagnosed phosphate-dependent disease or condition (such as hyperphosphatemia, hypophosphatemia, or the effects thereof) after ASARM peptide or ASARM inhibitor treatment indicates the efficaciousness of the treatment.

Delivery of Nucleic Acids Encoding MEPE and/or ASARM-Peptide to Tumor Cells

MEPE can be administered to patients (e.g., subjects with cancer) as a polypeptide, or can be administered as a nucleic acid that encodes MEPE. For example, as described herein, tumor cells can be removed from a patient and transfected ex vivo with an expression vector that is capable of driving MEPE expression in the tumor cells, after which the tumor cells are re-introduced into the patient prior to re-introduction into the patient, the tumor cells can optionally be irradiated to inhibit their ability to divide, as is well known in the art). Such tumor “vaccination” protocols enhance the ability of the immune system to destroy the tumors.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, et al., Science 247:1465-1468, 1990; and Wolff, Nature 352:815-818,1991. Such methods are well known in the art and readily adaptable for use with the compositions and methods described herein.

Inhibition of ASARM Biological Activity

The disclosed subject matter features methods of inhibiting the biological activity of ASARM to treat patients with diseases involving abnormally high levels of ASARM (e.g., but not limited to, X-linked hypophosphatemic rickets and oncogenic osteomalacia). Any compound can be used in the methods disclosed herein, as long as it inhibits ASARM biological activity, as described herein.

Antibodies

Antibodies can be used to inhibit ASARM biological activity. As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain ASARM binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. “Antibodies: A Laboratory Manual,” Cold Spring Harbor Publications, New York, (1988), which is incorporated by reference herein for its teachings of methods for producing and screening antibodies).

Also included within the meaning of “antibody or fragments thereof” are conjugates of antibody fragments and antigen binding proteins (single chain antibodies) as described, for example, in U.S. Pat. No. 4,704,692, which is incorporated by reference herein for its teachings of conjugates of antibody fragments and antigen binding proteins.

Also included within the meaning of antibody or fragments thereof” are fully human or humanized antibodies. Examples of techniques for human monoclonal antibody production include those described by Cole et al. (Monoclonal Antibodies and Cancer Therapy, Alan R. Liss, p. 77, 1985) and by Boemer, et al., Immunol 147:86-95, 1991. Human antibodies (and fragments thereof) can also be produced using phage display libraries (see Hoogenboom, et al., J Mol Biol 227:381, 1991; Marks, et al., J Mol Biol 222:581, 1991). Methods for humanizing non-human antibodies are well known in the art. For example, humanized antibodies can be generated according to the methods of Winter and co-workers (Jones, et al., Nature 321:522-525, 1986; Riechmann, et al., Nature 332:323-327, 1988; Verhoeyen, et al., Science 239:1534-1536, 1988), by substituting rodent CDRs or CDR sequences for the corresponding sequences of a human antibody. Methods that can be used to produce humanized antibodies are also described in U.S. Pat. Nos. 4,816,567, 5,565,332, 5,721,367, 5,837,243, 5, 939,598, 6,130,364, and 6,180,377.

Contemplated herein is a composition comprising a molecule that inhibits ASARM, wherein the molecule is an antibody. The antibody can bind to and inhibit the activity of any molecule associated with promoting ASARM biological activity, including ASARM itself, MEPE, cathepsin-B, or the renal or intestinal sodium-phosphate antiporter.

The antibodies can be either polyclonal or monoclonal.

Test Compounds

In general, compounds that modulate ASARM biological activity can be identified from large libraries of natural products or synthetic (or semi-synthetic) extracts or chemical libraries according to known assays and screening methods as described herein, and as will be apparent to the skilled artisan. Those skilled in the field of drug discovery and development will understand that the precise source of test extracts or compounds is not critical to the screening procedure(s) that allow identification of compounds that modulate ASARM biological activity. (By “compounds that modulate ASARM activity” is meant compounds that inhibit endogenous ASARM peptide production and/or activity, as well as compounds that stimulated endogenous ASARM peptide production and/or activity. Also included in this definition are ASARM mimetics, which can be administered to treat diseases and conditions involving abnormal phosphate metabolism, as described herein.) Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, polypeptides, small molecules, antibodies, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g. by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their ability to modulate ASARM activity should be employed whenever possible.

When a crude extract is found to have ASARM activity or ASARM inhibitory activity, for example, further fractionation of the positive lead extract is necessary to isolate chemical constituents responsible for the observed effect. Thus, the goal of the extraction, fractionation, and purification process is the careful characterization and identification of a chemical entity within the crude extract having an activity that mimics or antagonizes ASARM. The same assays described herein for the detection of activities in mixtures of compounds can be used to purify the active component and to test derivatives thereof. Methods of fractionation and purification of such heterogeneous extracts are known in the art. If desired, compounds shown to be useful agents for treatment are chemically modified according to methods known in the art. Compounds identified as being of therapeutic value can be subsequently analyzed using animal models for diseases or conditions in which it is desirable to regulate ASARM activity, as described herein.

### EXAMPLES

The disclosed subject matter is more particularly described in the following examples, which are intended as illustrative only since numerous modifications and variations thereof will be apparent to those of ordinary skill in the art.

### Example I

### MEPE Causes Hypophosphatemia and Modulates Mineralization

A. Materials and Methods

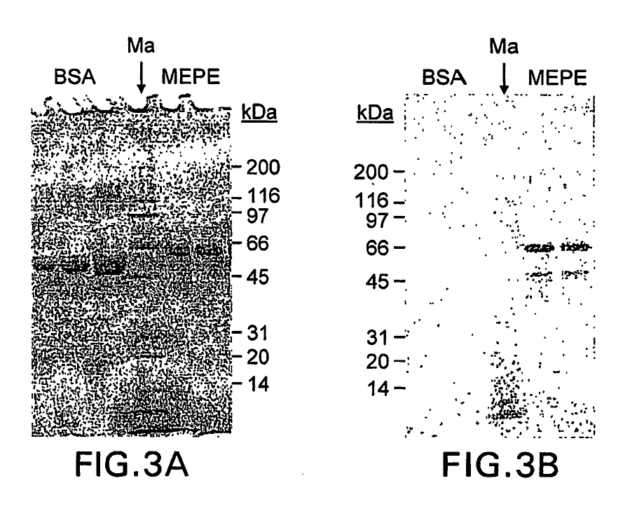

Recombinant Hu-MEPE. Insect S. frugiperda cells were infected with Baculovirus containing the human MEPE gene at a multiplicity of 1.0. Cells were grown in a 10 L bioreactor for 48 hours and conditioned media was concentrated (5 fold) and used as the starting material for purification. The purification scheme employed cation-exchange chromatography as the capture step followed by affinity chromatography and size-exclusion chromatography for final polishing and buffer exchange. The final product integrity was verified by N-terminal sequence analysis and Western blot analyses using antibodies to the N-terminus, mid-region and C-terminus. Purified protein contained N-terminal amino acid residues APTFQ confirming cleavage of predicted nascent-MEPE signal-peptide by the S. frugiperda insect cells. FIG. 3 shows SDS-PAGE (Coomassie stain) and Western blot of representative material used in these studies.

MEPE ASARM-peptide and polyclonal-antisera. The C-terminal MEPE ASARM-peptide (CFSSRRRDDSSESSDSGSSSESDGD; SEQ ID NO. 5) with NH₂-terminal cysteine was used as an experimental peptide and also to raise polyclonal antisera (FIG. 1). The following peptide was used as a control in the mineralization experiments (CGSGYTDLQERGDNDISPFSGDGQPF; SEQ ID NO. 6). Synthetic MEPE peptides corresponding to regions in the N-terminus (APTFQPQTEKTKQSC; SEQ ID NO. 10) mid region (TDLQERGDNDISPFSGDGQ; SEQ ID NO. 11) and C-terminal region (GRQPHSNRRFSSRRRDDSS; SEQ ID NO. 12) were also used to raise polyclonal antisera in rabbits. Antibodies to these peptides were affinity purified using the respective peptides and titred for optimal use in Western blot detection of MEPE. All peptides are oriented amino-terminal to carboxy-terminal.

Measurement of ³³PO₄ uptake and ¹⁴C-α-methyl D-glucose uptake. A novel technique, scintillation proximity assay (SPA), was used to measure ³³PO₄ uptake in-vitro. Human renal proximal-tubule primary cells (RPTEC) were purchased from Clonetics-Biowhittaker (Walkersville, Md.) (cat. no. CC2553) and were cultured and subcultured using the manufacturers recommended protocols, methods, buffers and media (cat. nos. CC3190, CC5034). Also a human renal cell-line (Hu-CL8) was cultured as previously described (Rowe et al. Candidate 56 and 58 kDa protein(s) responsible for mediating the renal defects in oncogenic hypophosphatemic osteomalacia. Bone 18(2):159-169, 1996). For ³³PO₄-uptake measurement cells were grown in REBM media supplemented with 5% fetal calf serum (REBM-FCS) to 80% confluence (Clonetics-Biowhitaker, Walkersville, Md.: cat. nos. CC2553, CC3190 and CC5034) and seeded into 96 well (3000 cells/well) cytostar-T scintillating-microplates (Amersham-Pharmacia Biotech, Piscataway, N.J.: cat. no. RPNQ 0162). Cells were then cultured in 200 μL of REBM-FCS media for 24 h. Media was then replaced with serum free media (REBM) and the cells incubated for a further 3-4 h with Hu-MEPE, PTH (1-34) or media control respectively in REBM media. The concentrations of PTH (1-34) and Hu-MEPE were 1 to 500 ng/nl and each experimental dose was repeated eight times for statistical analysis. Following incubation media was removed and replaced with either phosphate uptake buffer (PUB) consisting of 150 mM NaCl/1 mM Cacl2/1.8 mM MgSO₄/10 mM HEPE pH 7.0/0.1 mM phosphate buffer pH 7.0 and 3 uCi mL^{-1 33}P as orthophosphoric acid (18 MBq/mL or 5775.7 Gbq/mg stock solution Perkin Elmer, Wellesley, Mass.: cat. no. NEZ080). In order to determine the Na-dependent phosphate co-transporter activity a separate set of control cells were incubated with the same buffer but with choline chloride (150 mM) substituted for NaCl (CHUB buffer). After 10 min at 37° C. the reaction was terminated by removing the PUB and CHUB buffers and replacing with STOP buffer (CHUB buffer without radioisotope). The cytostar scintillation plates were then directly assayed for counts (CPM) using a Hewlett-Packard (Palo Alto, Calif.) Multiplate Top-counter calibrated for ³³P SPA assay. Due to the unique design of the 96 well cytostar-plate radioactive decay in solution is not detected by the counter. In contrast, radioisotope attached to or taken into the cells initiates release of plastic-scintillant photons that are detected by the top-counter photo-multiplier tube. Thus, no washes are required and direct CPM can be counted in living cells. Following CPM determination the buffers were removed from the living cells and fresh media supplemented with cell-titre-96 (Promega-Biotech, Madison, Wis.: cat. no. G5421) cell-number detection media. Cells were then further incubated at 37° C. for 4 h. Conversion of a tetrazolium salt to a colored formazan product by living cells was then measured at 490 nm using a 96 well multi-well spectrophotometer with KC-4 software (absorbance at 490 nM is directly proportional to the number of living cells). Na-dependent ³³P uptake was then calculated by subtracting background counts and the choline chloride non Na-dependent ³³P uptake and results expressed as ³³PO₄ femtomoles/10⁴-cells/6 min. The measurement of Na-dependent α-methyl D-glucose uptake was accomplished using a previously described method of ¹⁴C incorporation (Rowe, et al., Candidate 56 and 58 kDa protein(s) responsible for mediating the renal defects in oncogenic hypophosphatemic osteomalacia, Bone 18(2):159-169, 1996). The culture of cells and incubation with Hu-MEPE, PTH and controls were identical to the experiment for determination of phosphate uptake in RPTEC and Hu-CL8 cells.

Intraperitoneal administration of Hu-MEPE and PTH (1-34) into mice. Male ICR-Swiss mice were used as recipients for intraperitoneal administration (100 μl) of 0.1% phosphate-buffered saline/0.1% bovine serum albumin (vehicle) and experimental conditions (Hu-MEPE and PTH 1-34) dissolved in 100 μL of vehicle. Two separate (repeat) experiments were conducted consisting of four groups with 6 and 7 animals per group. The control group was administered with 100 μL of vehicle (Group-1). Group-2 consisted of animals administered with 0.8 μg/injection of parathyroid hormone (PTH 1-34). Group 3 were injected with 0.4 μg/injection of Hu-MEPE and Group-4 with 4 μg/injection of Hu-MEPE. On day one, three bolus injections were given at 0, 3 h, and 6 h. Serum samples were then collected via retro-orbital bleed one hour after the 6 h bolus administration. The next day or 30 hours after the first bolus a fourth bolus (same amount) was given and after 1 h the animals were sacrificed and serum prepared after cardiac exsanguination. The total amounts of PTH and MEPE administered over the 30 h were 80 μg/kg/30 h PTH 1-34, 40 μg/kg/30 h Hu-MEPE (MEPE-40) and 400 μg/kg/30 h Hu-MEPE (MEPE-400). Each group was placed in a separate metabolic cage and urine was collected for the 6-hour period. After the first 6-hour urine collection mice were replaced in thoroughly cleaned metabolic cages for the remaining 24 hours. The second 24-hour urine was collected immediately after the animals were sacrificed. The urine volumes for the second and first collections were measured. Collecting urine samples from large numbers of small mice over the time frame of the experiment was not feasible. Therefore, urine samples from each group (n=7) were pooled and volumes measured via a specially designed collecting duct in the metabolic cage. To ensure reproducibility the experiment was repeated twice (the same changes occurred). To calculate the fractional excretion of phosphate (FEP) the following equation was used: 100−(100×(1−(A/B))) where A=(urine phosphorus in mg/dL)×(serum creatinine in mg/dL) and B=(urine creatinine in mg/dL)×(serum phosphorus in mg/dL). To calculate renal phosphorus clearance (RPC) in mL/minute following computation was used: (urine phosphorus in mg/dL)×(urinary volume in mL)/((serum phosphorus in mg/dL)×(time of collection in minutes)). The phosphate excretion index (PEI) was calculated using the same method described by Shimada et al. (Cloning and characterization of FGF23 as a causative factor of tumor-induced osteomalacia, Proc Natl Acad Sci USA 98:6500-6505, 2001) using the following formula: ((urinary concentration of phosphorus in mg/dL)/(serum concentration of phosphorus in mg/dL))/(total urinary creatinine in mg).

Serum and urine assays. Serum and urine assays for phosphate, calcium, chloride, potassium, creatinine, glucose and alkaline phosphatase were accomplished using 96 well format KC4-spectrophotometer multiplate kinetic/end-point analyses using routine methods and diagnostic-kits Sigma-Diagnostics). Serum 1,25-dihydroxy vitamin-D₃ levels were estimated using the method of Reinhardt, et al. (A microassay for 1,25-dihydroxyvitamin D not requiring high performance liquid chromatography: application to clinical studies, J Clin Endocrinol Metab 58:91-98, 1984). Analysis of data for fractional excretion of phosphate (FEP) was carried out following the same method described previously by Shimada et al. (Mutant FGF-23 responsible for autosomal dominant hypophosphatemic rickets is resistant to proteolytic cleavage and causes hypophosphatemia in vivo, Endocrinology 143:3179-82, 2002) for FGF23 bolus administration studies and by Nesbitt et al. (Crosstransplantation of kidneys in normal and Hyp mice: Evidence that the Hyp mouse phenotype is unrelated to an intrinsic renal defect, J Clin Invest 89:1453-59, 1992) for renal cross-transplantation experiments in Hyp mice.

Mineralized bone matrix formation assay. A mouse osteoblast cell-line (2T3-cells) were monitored using a mineralized matrix formation assay as described by Bhargava et al. (Ultrastructural analysis of bone nodules formed in vitro by isolated fetal rat calvaria cells. Bone 9:155-63, 1988) and cultured as described previously (Bhargava et al., supra; Chen et al. Differential roles for bone morphogenetic protein (BMP) receptor type IB and IA in differentiation and specification of mesenchymal precursor cells to osteoblast and adipocyte lineages. J Cell Biol 142:295-305, 1998; and Zhao, et al. Bone morphogenetic protein receptor signaling is necessary for normal murine postnatal bone formation. J Cell Biol 157:1049-60, 2002). Mineralization of the murine osteoblastic 2T3 cell-line has been extensively studied using physical techniques, electron microscopy and histological techniques. This cell line (2T3) undergoes exogenous BMP2-induced mineralized-matrix indistinguishable from normal bone-mineral hydroxyapatite in-vitro (Bhargava et al., supra; Chen et al., supra; and Zhao et al., supra). The cells were plated in 24-well culture plates at density of 2×10⁴ cells/well and cultured with minimal essential medium (MEM) supplemented with 10% FCS. When the cells reached confluency (day 0), the medium was changed to MEM containing 5% FCS, 100 μg/mL ascorbic acid and 5 mM β-glycerol phosphate with or without 100 ng/mL of BMP-2. The effects of Hu-MEPE, MEPE ASARM-peptide and control peptide at different doses (see results section) in the absence and presence of BMP2 were measured using the von Kossa stain for mineralization. The medium was changed every other day and fresh reagents were added. Von Kossa stain of mineralized bone matrix was performed as follows. The cell cultures were washed with PBS twice, fixed in phosphate buffered formalin for 10 min, and then washed with water, and serially dehydrated in 70%, 95%, and 100% ethanol, twice each and air dried. The plates were rehydrated from 100% to 95% to 80% ethanol to water before staining. The water was removed, a 2% silver nitrate solution was added, and the plates were exposed to sunlight for 20 min, after which the plates were rinsed with water. 5% sodium thiosulfate was added for 3 min and the plates were then rinsed with water. The modified van Gieson stain was then used as a counterstain after the von Kossa stain. The unmineralized collagen matrix can be recognized by the yellow/red van Gieson stain. The acid fuchsin solution (5 part of 1% acid fuchsin, 95 part of picric acid, and 0.25 part of 12 M HCl) was added for 5 min. The plates were washed with water, and then 2×95% ethanol and 2×100% ethanol, and dried for image analysis. The area of von Kossa stained matrix was quantitated as previously described (Chen et al., supra). Briefly matrix was quantified by automated image analysis using a video analysis program (Jandel Scientific, San Rafael Calif.) and also a Bio-Rad (Hercules, Calif.) Quantity-1 image analysis capture program. A video screen camera (CCD/RGB; Sony Corp., Park Ridge, N.J.) linked to a microscope (model BH2; Olympus Corp., Precision Instruments Division, Lake Success, N.Y.) equipped with metallurgical lens was used to image plates.

SDS-polyacrylamide electrophoresis (SDS-PAGE) and Western analysis. Proteins were separated and visualized using 4-12% SDS-PAGE Novex gel gradient and Coomassie blue staining as described previously (Rowe et al. MEPE, a new gene expressed in bone-marrow and tumors causing osteomalacia. Genomics 67 (1):54-68, 2000). Polyclonal antisera raised in rabbits were used to screen SDS-PAGE separated proteins electro-transferred and immobilized onto PVDF membranes (Western-blotting) using methods also previously described (Rowe et al., supra).

Statistical methods. Differences were assessed statistically by the use of Newman-Keuls or Bonferroni (as indicated) multiple comparison equations after one-way analysis of variance (non-parametric). A p value of less than 0.05 was considered significant. The standard error of the mean (SEM was used as a representative measure of how far the sample mean differed from the true population mean.

B. Results

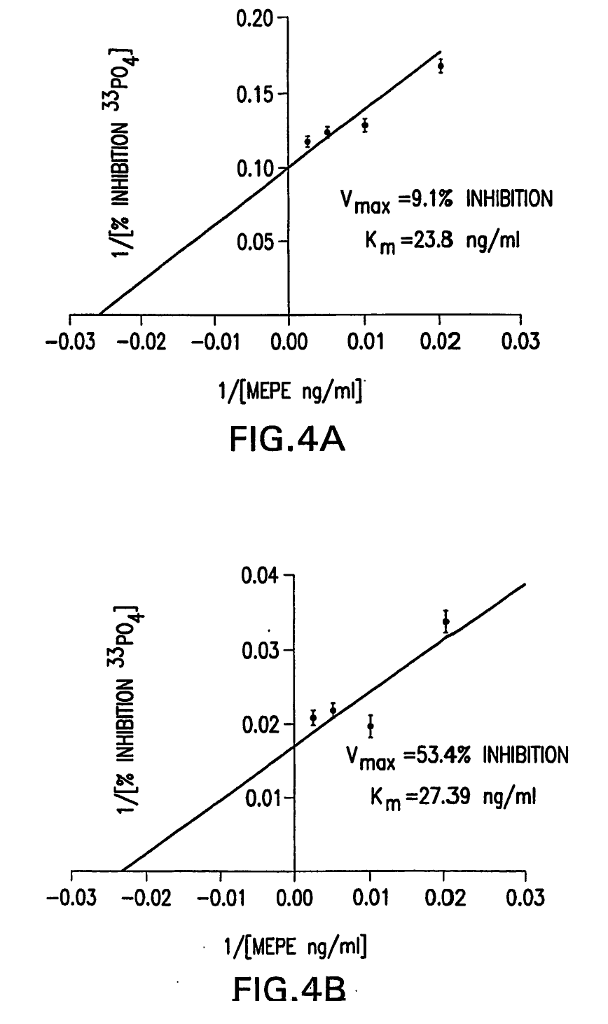

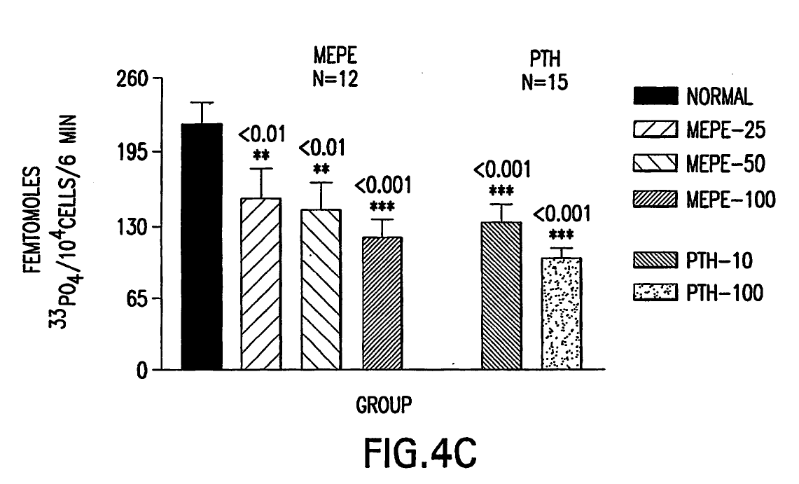

Measurement of ³³PO₄ uptake and ¹⁴C-α-methyl D-glucose uptake (SPA). Dose dependent inhibition of Na⁺ dependent phosphate co-transport occurred in both primary human renal proximal tubule epithelial cells (RPTEC) and a human renal proximal tubule cell-line (Hu-CL8) with PTH (1-34) and Hu-MEPE (FIGS. 4A-4C). Although the Km for Hu-MEPE was similar with RPTEC (27.4 ng/mL) and Hu-CL8 cells (23.8 ng/mL) the Vₘₐₓ of percentage inhibition differed significantly. Primary RPTEC cells exhibited a Vₘₐₓ of 53.4% inhibition and Hu-CL8 cells a lowered Vₘₐₓ of 9.1% inhibition (FIGS. 4a & b). FIG. 4c graphically (histogram) illustrates the dose-dependent inhibitory effects of Hu-MEPE and PTH (1-34) on RPTEC cells. No significant differences were noted in the Na-dependent uptake of ¹⁴C-α-methyl D-glucose uptake between controls and experimentals.

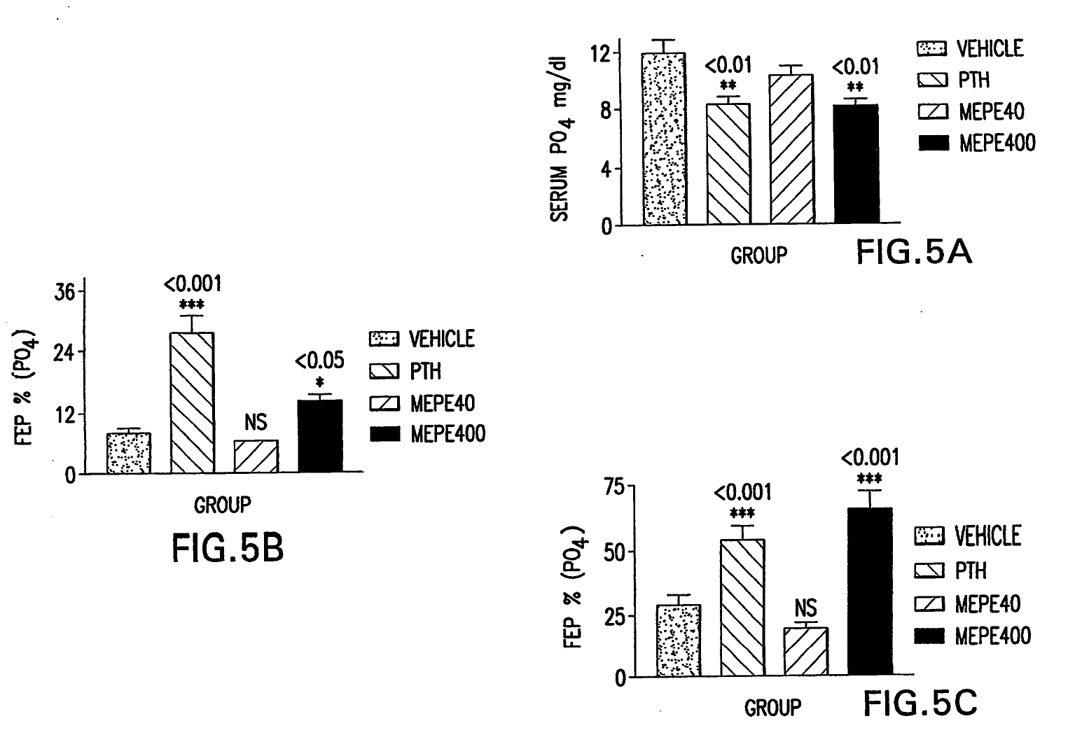

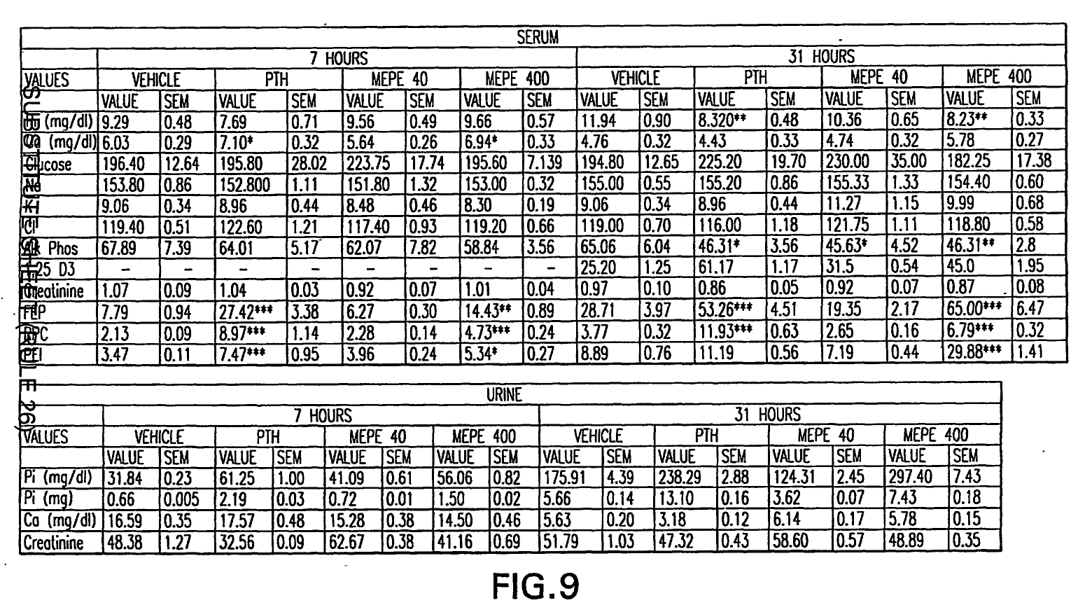

Intraperitoneal administration of Hu-MEPE and PTH (1-34) into mice. There were no significant differences in serum phosphate between vehicle, PTH, and MEPE animals after 7 hours. However, a marked and significant reduction in serum phosphate relative to vehicle (11.94 mg/dL; SEM 0.9) occurred in the PTH (8.32 mg/dL; SEM 0.48; P<0.01) animals and the high dose MEPE-400 (8.23 mg/dL; SEM 0.334; P<0.01) group after 31 hours (one hour after final bolus administration). A drop in serum phosphate in the low dose MEPE-40 animals (10.6 mg/dL; SEM 0.65) was also observed relative to vehicle after 31 h but this was not statistically significant (FIG. 5a). Urine phosphate increased dose dependently relative to vehicle (31.8 mg/dL; SEM 0.23) in PTH (61.6 mg/dL; SEM 1.0), MEPE-40 (41.0 mg/dL; SEM 0.61) and MEPE-400 (56.1 mg/dL; SEM 0.82) groups after 7 h dramatically, and reproducibly (FIG. 9). Also consistent with the observed hypophosphatemia in PTH and MEPE mice the 31 h second urine phosphate excretion was also markedly and reproducibly elevated in PTH mice (238.3 mg/dL; SEM 2.88) and MEPE400 (297.4 mg/dL; SEM 7.43) mice relative to vehicle (175.9 mg/dL; SEM 4.39) (FIG. 9). In contrast to the 7 h collections the 31 h low-dose MEPE-40 urine was not significantly different to the 31 h vehicle. This was not surprising as the smaller change elicited by the low Hu-MEPE dose was probably diluted by the overnight collection of urine (24-hours since last low dose Hu-MEPE bolus). The dose-dependent MEPE and PTH hyperphosphaturia was reproducible and occurred after three bolus injections of Hu-MEPE and PTH at 0, 3, and 6 h and was also markedly evident in the second overnight urine collection. Thus, the administration of Hu-MEPE and PTH resulted in hypophosphatemia and hyperphosphaturia.

Although the hypophosphatemia was manifest in PTH and Hu-MEPE groups after 31-h but not after 7-h, hyperphosphaturia was apparent dose dependently after 7 h and also after 31 h. Moreover, measurement of serum/urine creatinine, phosphate, urine volume and calculation of the fractional excretion of phosphate (FEP) together with renal phosphorus clearance (RPC) confirmed a dramatic and dose dependent loss of phosphate after 7-h and also 31-h (FIG. 9 and FIG. 5b-5c). This loss was underlined by a dramatic and reproducible increase in FEP (after 7 h) relative to vehicle (7.8%; SEM 0.94), in PTH (27.4%; SEM 3.38, <0.001) and MEPE-400 (14.43%; SEM 0.99, P<0.01) animals (FIG. 5b). Also the dramatic increase in FEP relative to vehicle (28.7%; SEM 3.97) was still evident in the PTH (53.3%; SEM 4.51, P<0.01) and MEPE-400 (65.0%; SEM 6.47, P<0.001) after 31 h (FIG. 5c). The low dose MEPE-40 group was not significantly different in FEP to the vehicle confirming a dose-dependent Hu-MEPE phosphaturic effect. The levels of serum calcium were significantly elevated in the PTH animals after 7 hours (one hour after third bolus administration). The low Hu-MEPE dose and vehicle dose were not significantly different. There were no differences in serum calcium between groups after 31 hours (single bolus second day administration). Although urine calcium was not significantly different to vehicle in all groups after 7-h the PTH animals were significantly hypocalciuric after 31 hours compared to Hu-MEPE and vehicle (FIG. 9). Serum values for a range of parameters including K, Cl, Na, glucose, protein, and albumin were not significantly different between groups (FIG. 9).

As expected the serum levels of 1,25-dihydroxy vitamin-D₃ in the PTH 1-34 animals (61.2 pg/mL) were reproducibly elevated after 31 h relative to vehicle (25.2 pg/mL). A dose dependent increase of 1,25-dihydroxy vitamin-D₃ also occurred reproducibly with Hu-MEPE with values of 31.5 pg/mL and 45.05 pg/mL for MEPE-40 and MEPE-400 respectively (FIG. 9). Insufficient serum was obtained via retro-orbital bleed to assay the 7-h time point. Serum alkaline phosphatase was not significantly different between vehicle and other groups after 7-h. By contrast, a highly significant and reproducible suppression of serum alkaline phosphatase was observed after 31 h between vehicle (65.06 U/L SEM 4.4) and MEPE-40 (45.63 U/L; SEM 4.52, P<0.05), MEPE-400 (46.31 U/L; SEM 2.8, P<0.01) and PTH (46.3 U/L; SEM 3.56, P<0.01) groups (FIG. 9).

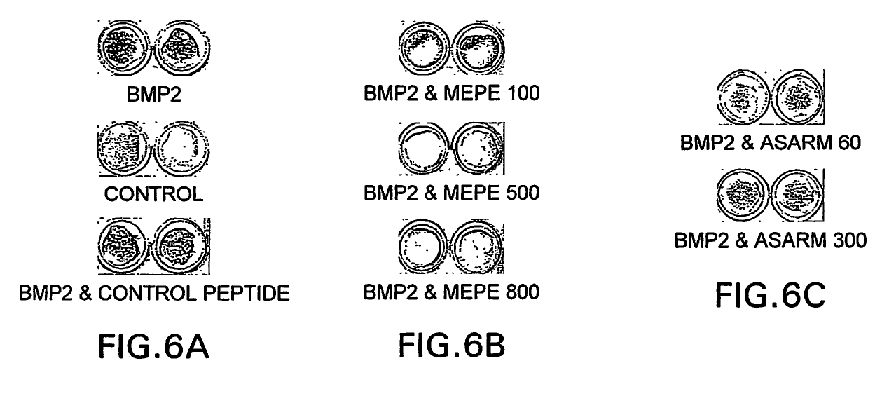

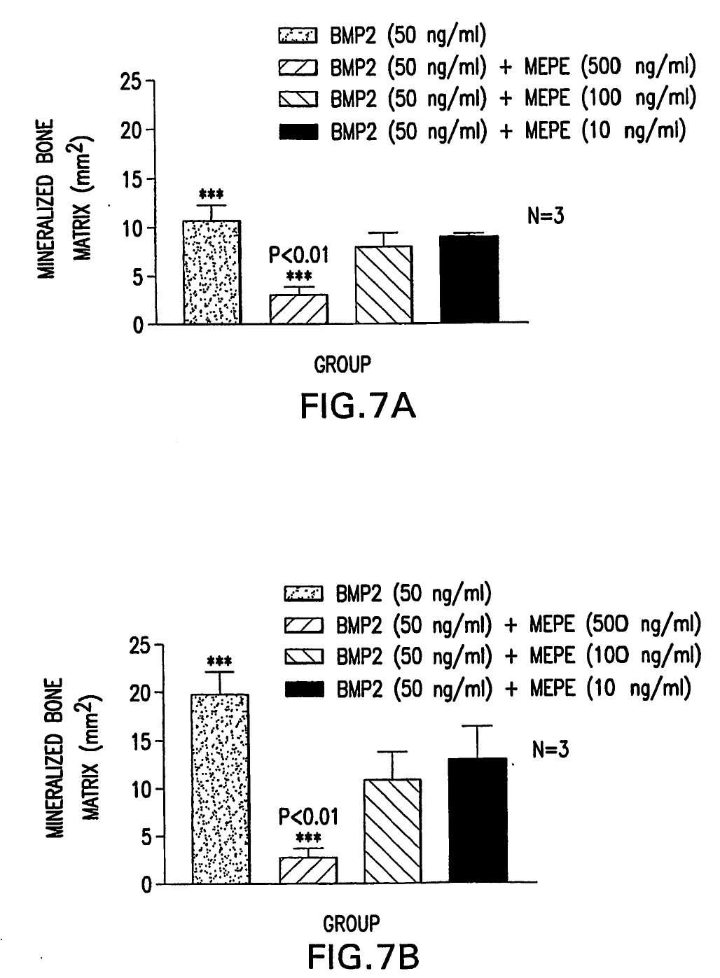

Mineralized bone matrix formation assay and effects of Hu-MEPE and MEPE ASARM-Reptide. BMP2 (100 ng/mL) induced mineralization of 2T3 osteoblasts as assayed using the von Kossa stain after 12 days. No von Kossa positive staining was apparent in the control cells without BMP2 up to 31 days. The results for 26 days are shown in FIG. 6 and FIG. 7 shows quantification of nodule formation after days 13 and 20 respectively. Addition of Hu-MEPE at concentrations of 10, 100, 500, and 800 ng/mL in the presence of BMP2 (100 ng/mL) dose dependently and reproducibly inhibited mineralization of 2T3 osteoblasts (FIGS. 6A-6B and 7A-7B). Doses above 100 ng/mL were very effective inhibitors. MEPE in the absence of BMP2 had no effect on mineralization and cells were identical to controls confirming an inhibitory role for MEPE or derived MEPE-peptide(s). Addition of the MEPE ASARM-peptide (DDSSESSDSGSSSESDGD; SEQ ID NO. 13) also dose-dependently inhibits BMP2 mediated von Kossa staining and bone mineral nodule formation in 2T3 cells (FIGS. 6A and 6C). A control peptide failed to elicit any changes in mineralization (positive or negative); see FIG. 6A. Quantification of the inhibitory effects of Hu-MEPE is shown in FIG. 7 for days 13 and 20.

### Example II

### Dosages of ASARM Peptide Affect Fractional Excretion of Phosphate

A. Materials and Methods

Four groups of 7 mice were injected subcutaneously twice a day for three weeks. The first group received vehicle only as a control, the second group received etidronate at 60 mg/kg/day, the third group received ASARM peptide at 1.3 mg/kg/day (“low ASARM”), and the fourth group received ASARM peptide at 4 mg/kg/day (“high ASARM”). Calcein/tetracycline was administered to the animals 4 days and 2 days before the end of the protocol for visualization purposes.

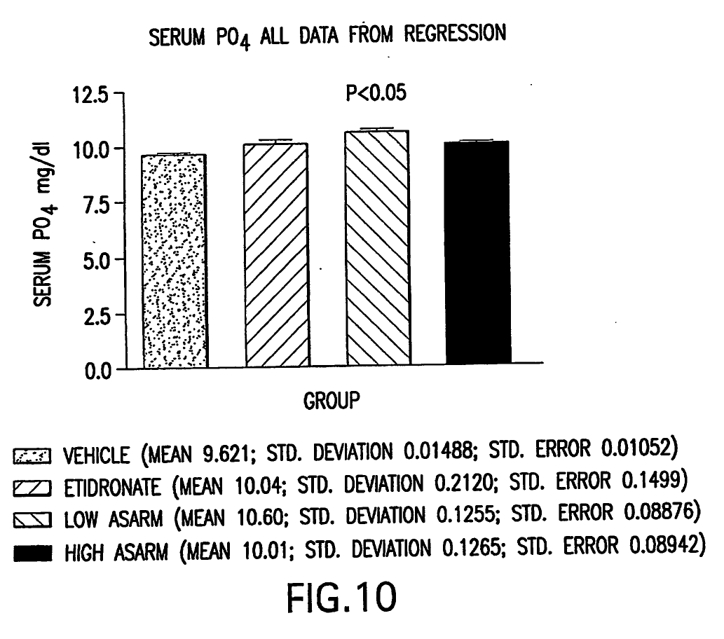

Serum phosphate levels were measured for all four groups (FIG. 10). The vehicle group had a mean level of 9.62 mg/dL, the etidronate group had a mean level of 10.04 mg/dl, the low ASARM group had a mean level 10.60 mg/dL, and the high ASARM group had a mean level of 10.01 mg/dL.

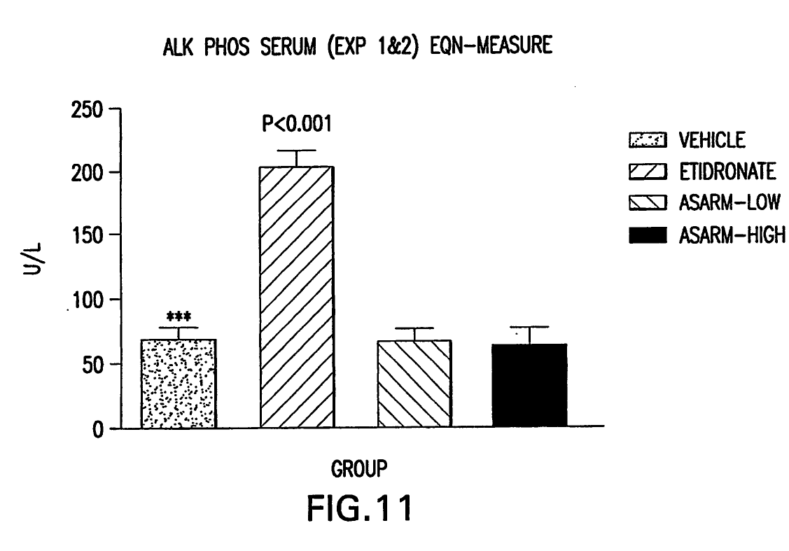

Alkaline phosphatase levels in serum were also measured for all four groups and compared using Bonferroni's multiple comparison test (FIG. 11). The etidronate caused significantly higher levels of alkaline phosphate in the serum, while both ASARM groups remained at levels similar to that of the vehicle (control).

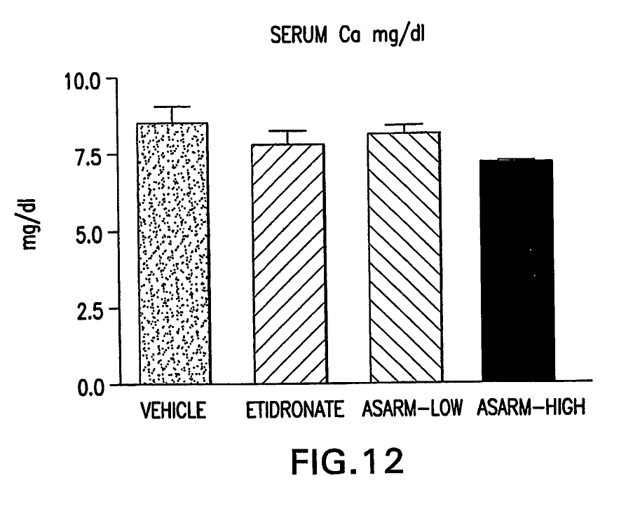

Serum calcium levels were also measured (FIG. 12). The mice given etidronate, low ASARM dosage, and high ASARM dosage all displayed lower serum calcium levels than the vehicle (control).

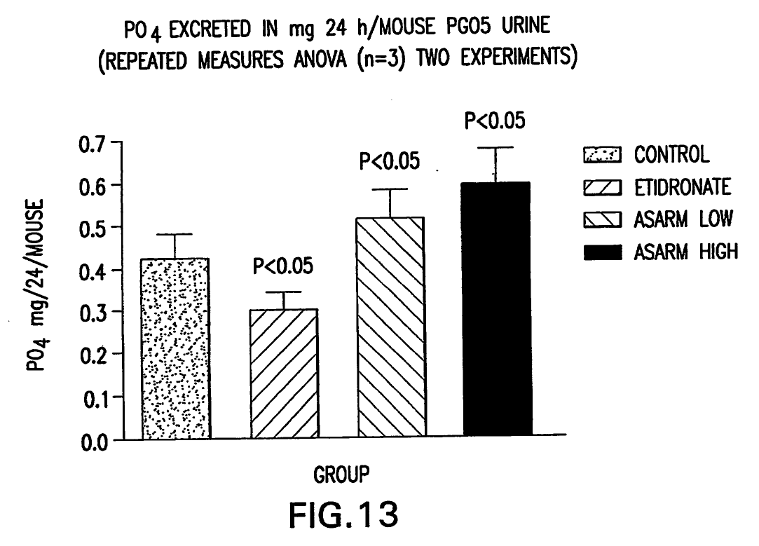

Phosphate excretion in urine of the mice was measured and compared using Newman-Keuls multiple comparison (FIG. 13). Urine was collected from the groups over a 24-hour period and phosphate levels measured. Urinary phosphate excretion was significantly higher in the low ASARM and high ASARM dosage groups, compared the etidronate and control groups.

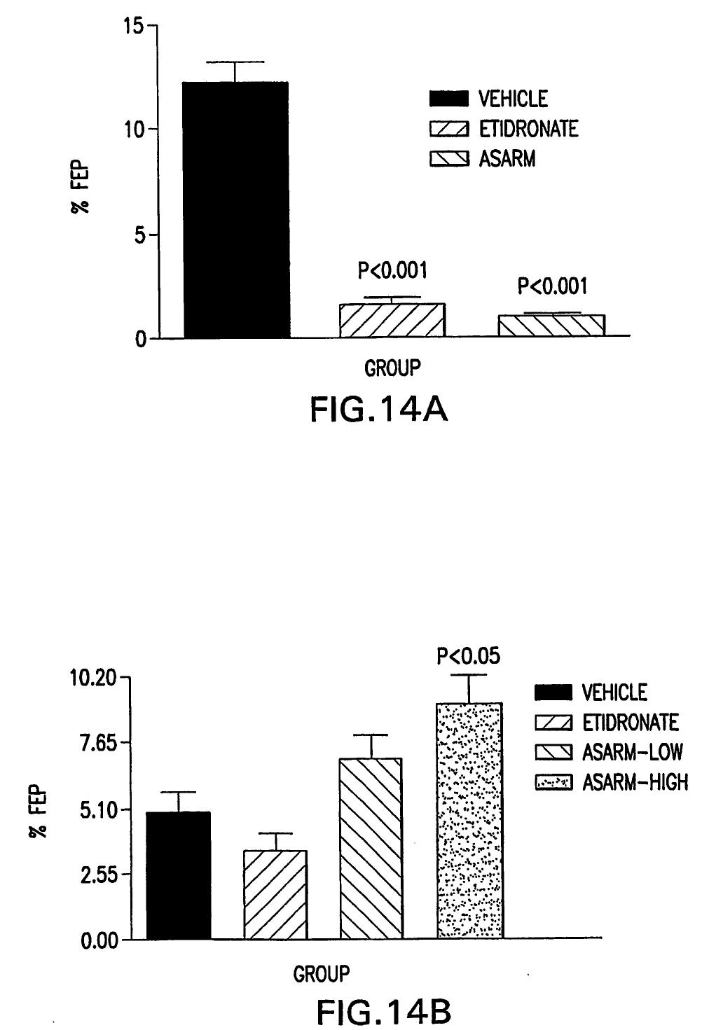

FIGS. 14a and 14b show the Fractional Excretion of Phosphate (FEP) in two different experiments. In the experiment shown in FIG. 14a, each of three groups of mice was treated with one injection per day, over two weeks, of either: (a) vehicle alone, (b) etidronate (10 mg/kg/day), or (c) ASARM peptide (2 mg/kg/day). In the experiment show in FIG. 14b, each of four groups of mice was treated with two injections per day, over three weeks, of either: (a) vehicle alone, (b) etidronate (60 mg/kg/day), (c) ASARM (1.34 mg/kg/day; “ASARM-low”), or (d) ASARM (4.00 mg/kg/day; “ASARM-high”).

In the experiment in which mice received one injection per day, the animals given ASARM peptide showed a significantly lower % FEP than the control group, and a slightly lower % FEP than the etidronate group. In the experiment in which mice received two injections per day, both the low ASARM and the high ASARM animals showed much higher % FEP levels than the control animals and the etidronate animals.

B. Results

A single bolus of ASARM peptide injection per day results in a decrease in FEP over a two week period. In contrast, the animals that were given two boluses of ASARM peptide per day over three weeks showed in an increase in FEP, showing that, under such saturating conditions, poisoning of the sodium/phosphate antiporter by ASARM can occur. This is consistent with direct binding of ASARM peptide to NPT2. Such dual effects have been reported for phosphonoformic acid, but phosphonoformic acid, unlike ASARM peptide, has been established as being too toxic for medical use.

### Example III

### ASARM can Induce Hyperphosphatemia and Inhibit Bone Mineralization

A. Materials and Methods

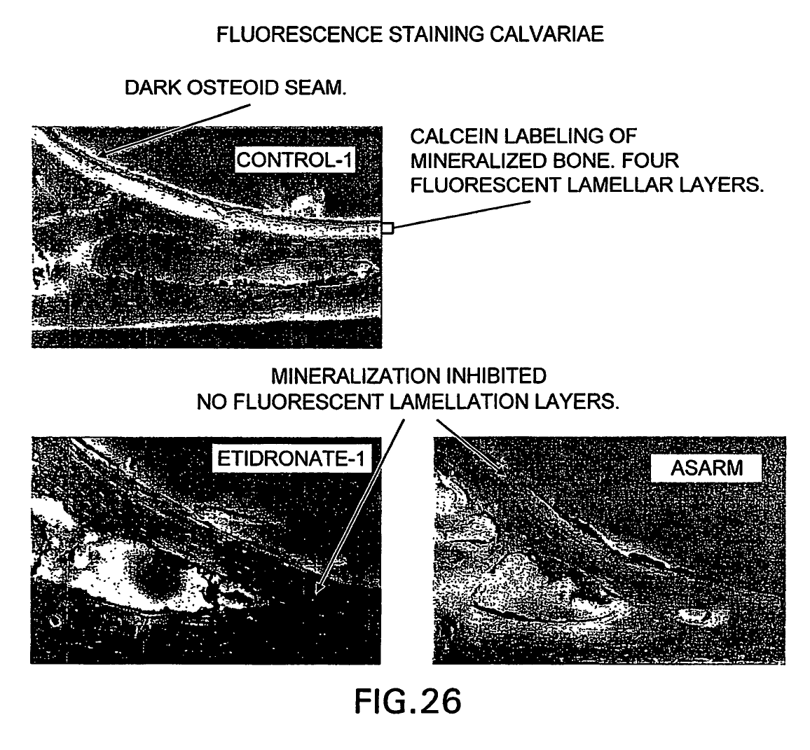

Three groups of 5 mice each received a subcutaneous injection once a day for 12 days. The mice did not receive treatment for 2 of these 12 days. Calcein was given at 20 mg/kg/day on days 3, 5, 8, and 10 to allow visualization. The first group of mice were given an injection of vehicle only. The second group of mice were given etidronate at 10 mg/kg/day. The third group of mice were given ASARM peptide at 2 mg/kg/day. Mineralization was greatly inhibited in the calvariae and long bones of the mice given etidronate and ASARM, but not in those given vehicle only. Using one way ANOVA (non-parametric) Neuman-Keuls multiple comparison, mineralization of calvariae (adj. vol CNT/mm²), 48.4% inhibition of mineralization was seen in those mice given etidronate, and 62.0% inhibition of mineralization was seen in those mice given ASARM peptide. Furthermore, in fluorescent staining in calvariae, mineralization is inhibited in both the etidronate and ASARM-treated calvariae, while the control displays a dark osteoid seam, showing that mineralization occurred (FIG. 26).

Inhibition of mineralization also occurred in the tibia/femur of those mice given ASARM peptide. 24.4% inhibition occurred in the tibia/femur of mice given ASARM peptide, and 19.2% inhibition occurred in the tibia/femur of mice given etidronate. 44.6% inhibition was seen in the femur of mice given ASARM peptide, and 15.8% inhibition was seen in the femur of mice given etidronate. 35.3% inhibition in tooth mineralization was seen in mice given ASARM peptide, and 53.2% inhibition was seen in mice given etidronate. No significant inhibition of mineralization was seen in any of the mice given vehicle only.

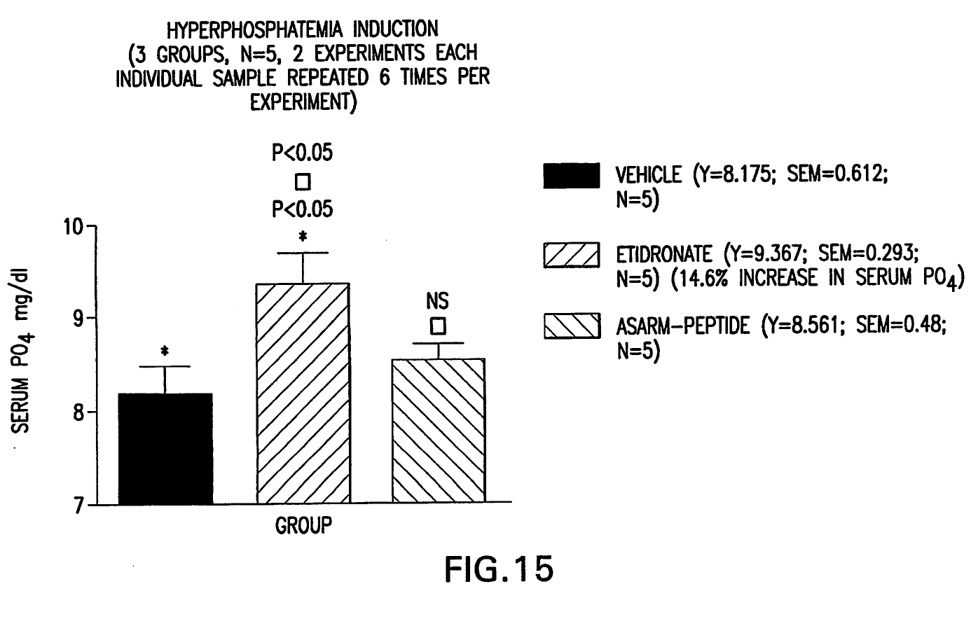

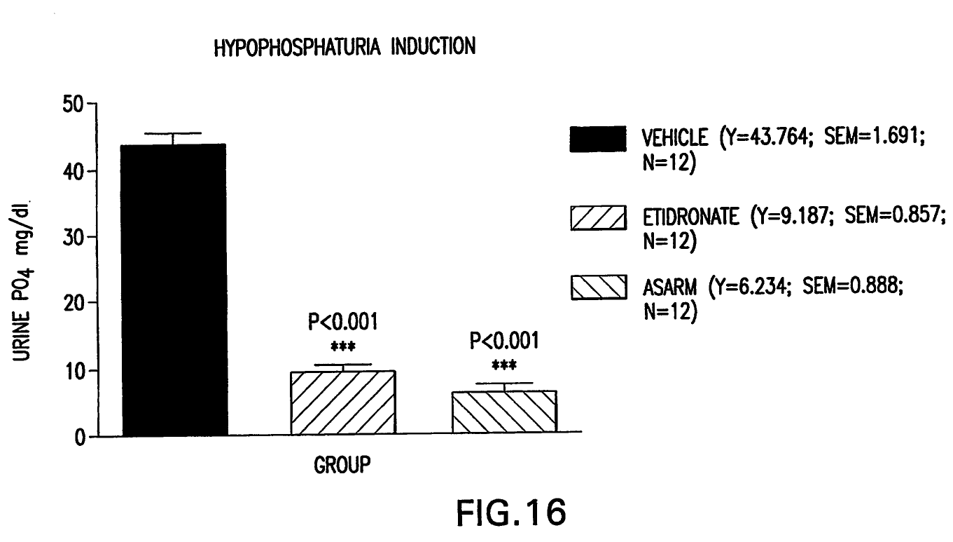

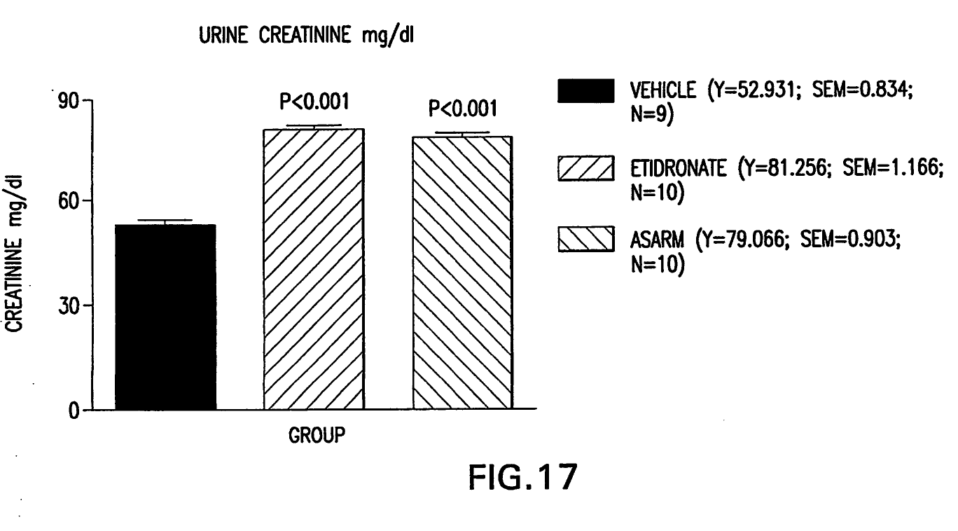

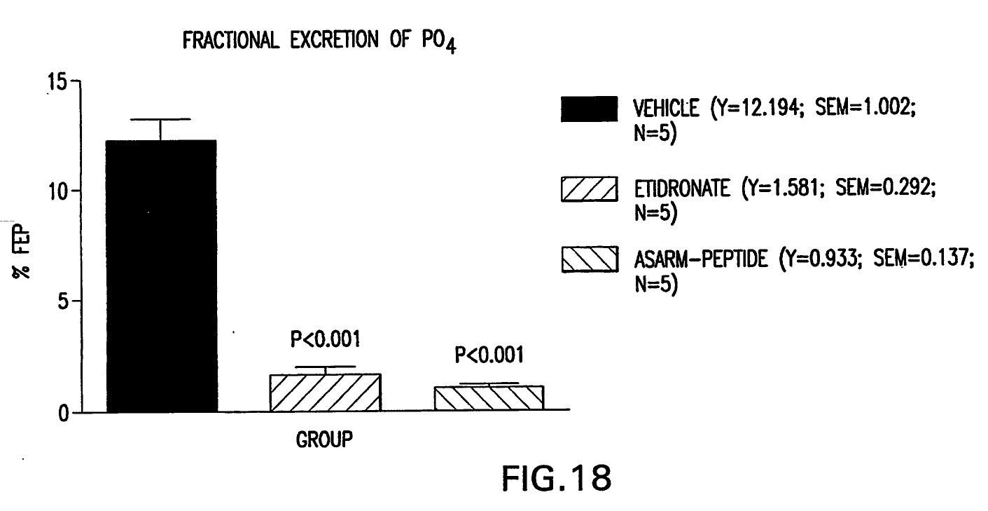

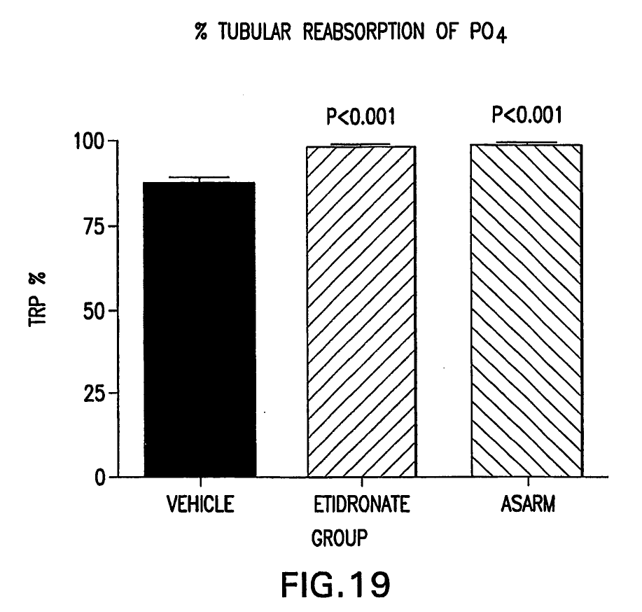

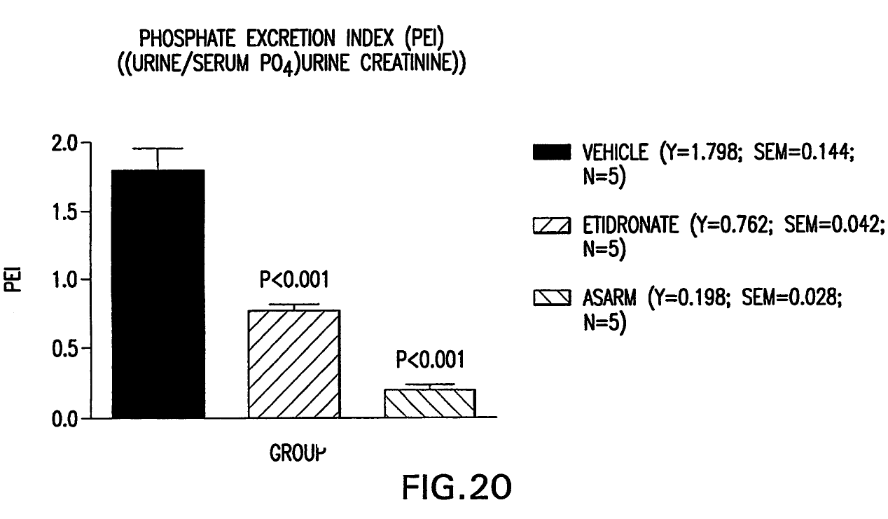

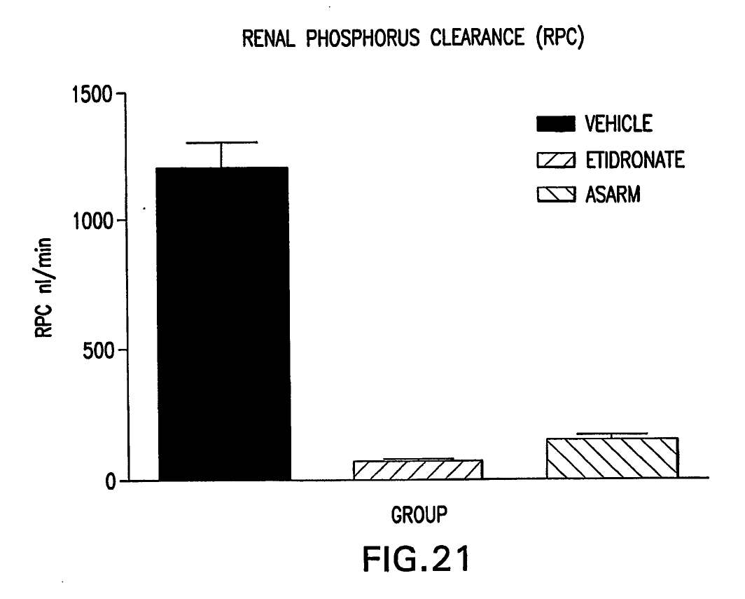

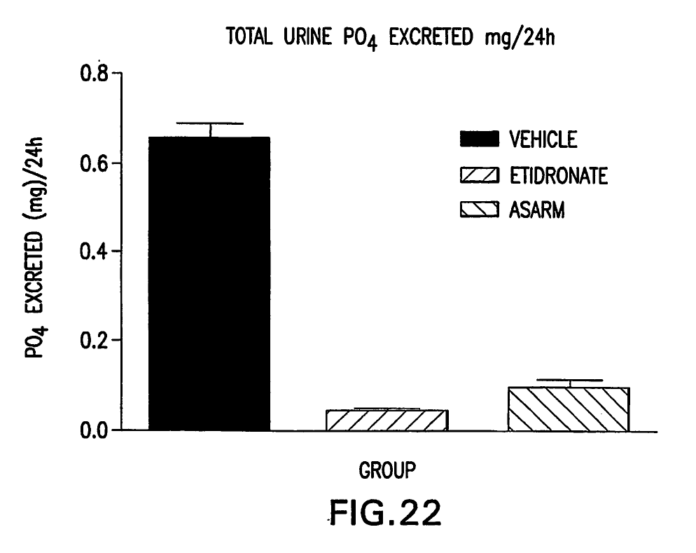

ASARM was found to induce hyperphosphatemia (FIG. 15). Furthermore, both etidronate and ASARM peptide also induced hypophosphaturia, as seen by the Neuman-Keuls multiple comparison test (FIG. 16). Both etidronate and ASARM peptide also induced significant elevation of urine creatinine in treated mice (FIG. 17). Fractional Excretion of Phosphate (FEP) was significantly lower in both the etidronate-treated and ASARM-treated groups compared with the vehicle-treated group (FIG. 18). Tubular reabsorption of phosphate was also higher in the etidronate-treated and ASARM-treated groups compared with the vehicle-treated group, as measured by percent tubular reabsorption of phosphate (FIG. 19). The Phosphate Excretion Index (PEI) for each of the three groups was calculated. The PEI measures urine/serum phosphate and urine creatinine (FIG. 20). ASARM-treated mice displayed a much lower PEI than either etidronate-treated or vehicle-treated mice groups. Renal phosphorous clearance was also measured. ASARM-treated mice and etidronate-treated mice displayed significantly less clearance than vehicle-treated mice (FIG. 21). Moreover, both etidronate-treated and ASARM-treated groups excreted far less urinary phosphate in a 24-hour period than did the control group (FIG. 22).

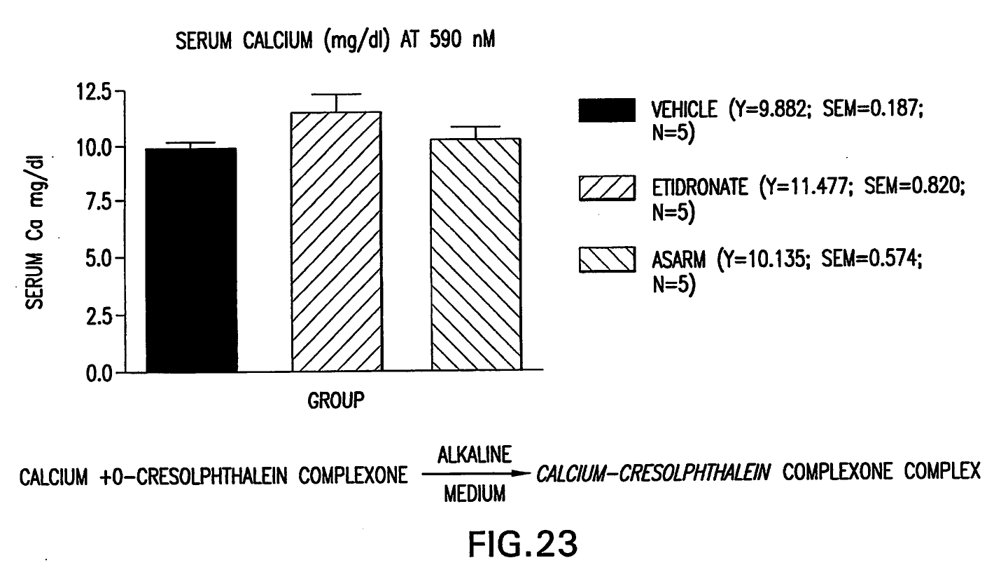

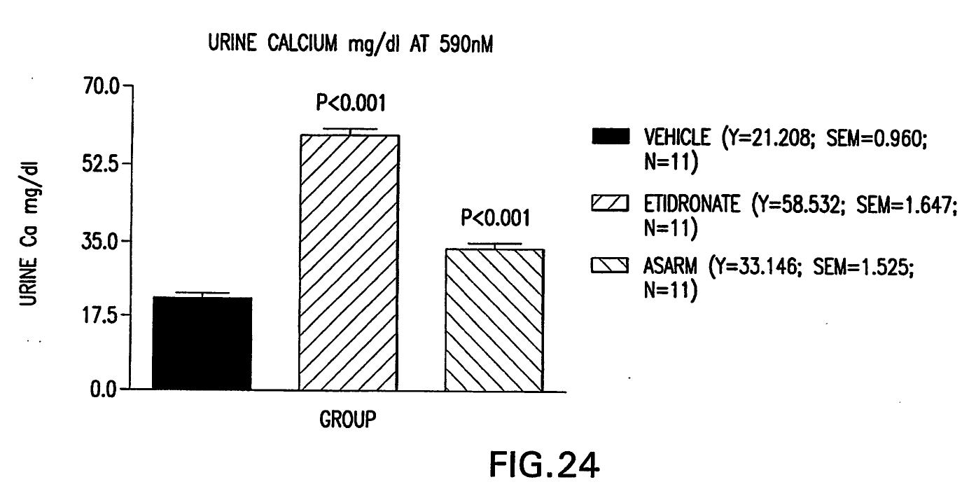

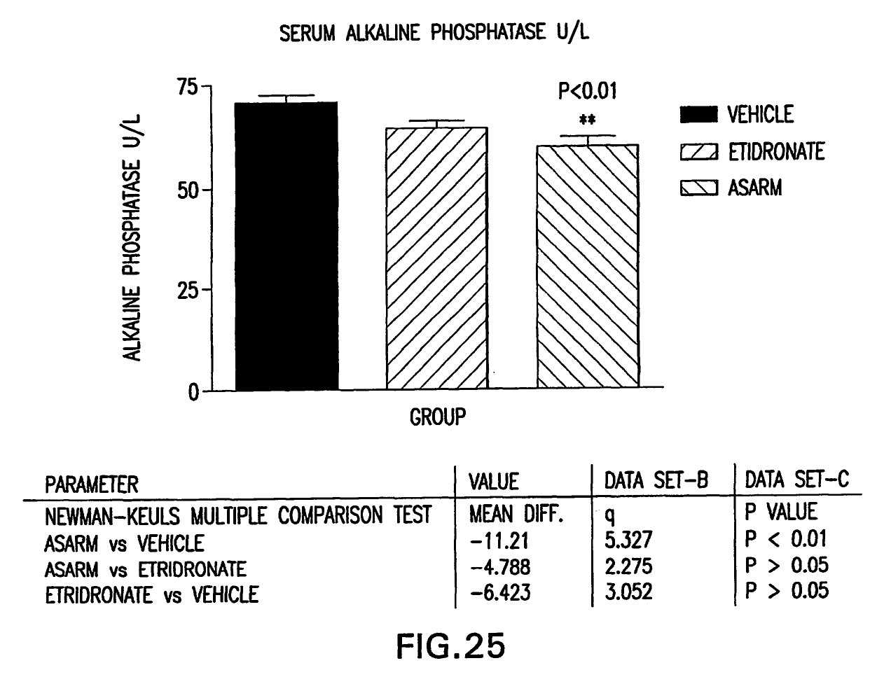

Serum calcium was also measured in mg/dl at 590 nM (FIG. 23). The ASARM-treated group did not show a significantly elevated level of serum calcium compared to the control, whereas the etidronate-treated group showed a slightly higher level of serum calcium. Urine calcium levels were also measured (FIG. 24) in mg/dl at 590 nM. Whereas the etidronate group showed a 75.9% increase in urinary calcium levels compared to the control, the ASARM group showed a more modest 56.3% increase in urinary calcium levels compared to the control. In addition, the ASARM group displayed lower levels of serum alkaline phosphatase than did the etidronate or vehicle groups.

B. Results

Under the conditions of the experiments described above, mineralization was inhibited (as measured by the quenching of calcein-fluorescence) in calvariae, femur/tibia, and teeth of mice given ASARM peptide. Under these experimental conditions, ASARM induced hyperphosphatemia (an elevation in serum phosphate) and hypophosphaturia (a decrease in urinary phosphorus), and decreased the Fractional Excretion of Phosphate (FEP).

### Example IV

### Growth Inhibition CHO Cell Tumors that Express MEPE

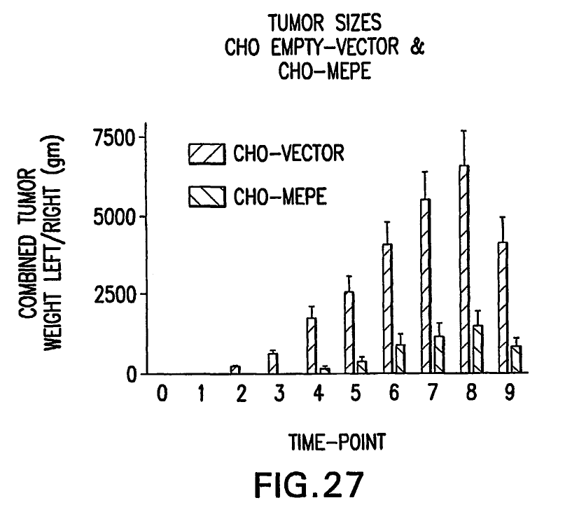

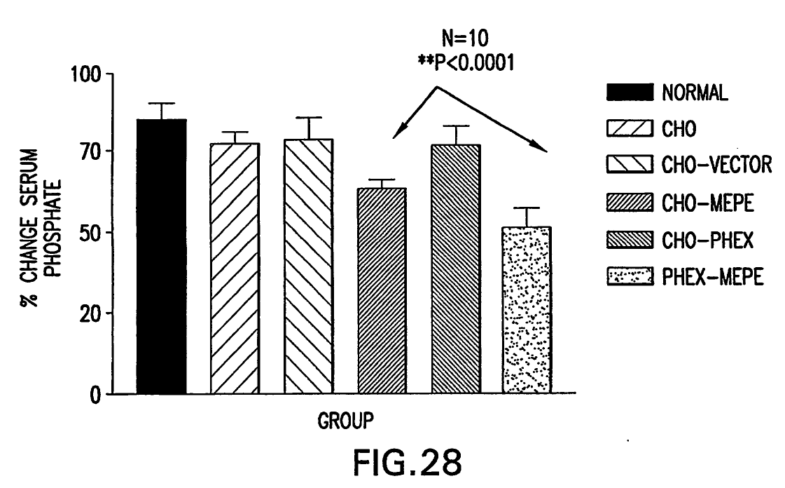

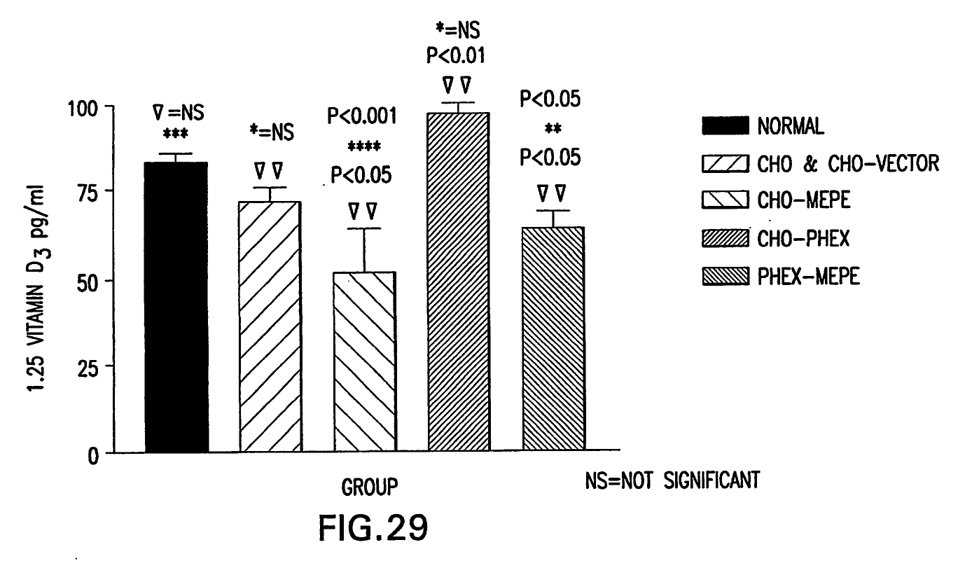

Six groups of athymic nude mice were injected with the one of the following: normal saline, CHO cells, CHO cells containing an empty vector, CHO cells expressing MEPE (CHO-MEPE), CHO cells expressing PHEX (CHO-PHEX), and CHO cells expressing PHEX and MEPE (CHO-PHEX-MEPE). Tumor size was assessed based on combined tumor weight at nine different time points. As shown in FIG. 27, tumor size was significantly reduced in the mice containing CHO cells expressing MEPE as compared to the mice containing CHO cells with an empty vector. Furthermore, those mice expressing MEPE (both CHO-MEPE and CHO-PHEX-MEPE groups) showed a significant percent change in serum phosphate levels after thirteen days, showing hypophosphatemia in mice expressing MEPE as compared to the other four groups (FIG. 28). Also, those mice expressing MEPE (both CHO-MEPE and CHO-PHEX-MEPE groups) showed a decrease in 1,25-vitamin D3 serum levels as compared to the four groups of mice not expressing MEPE (FIG. 29).

### Example V

### Immunodetection of ASARM Peptide in Patient with X-Linked Hypophosphatemic Rickets (HYP)

The serum of a patient with X-linked hypophosphatemic rickets was subjected to solid phase extraction for al peptides less than 3 kDa using HPLC C18 RP. A signal at the position found to be positive for synthetic ASARM peptide was detected. To further analyze these results, Western blotting was used to detect endogenous ASARM peptide using a specific anti-ASARM antibody, confirming that the patient was immunopositive for the ASARM peptide.

Furthermore, immunodensitometric quantification of the ASARM peptide was performed in serum obtained from the father of the patient, who was unaffected by the disease. Using chemiluminescence and FluorImager^{max} (Bio-Rad, Hercules, Calif.), the results were negative for the father, showing that the peak-1 regions and peak-2 regions were relatively the same as the control when 1 ml of serum was assayed for ASARM peptide.

### Example VI

### MEPE Binds to PHEX via the MEPE-ASARM Motif: Surface Plasmon Resonance

A. Materials and Methods

Expression of insect-expressed MEPE and soluble mammalian expressed PHEX (secPHEX). Expression and purification of full-length insect-expressed human-MEPE was as described previously in Example 1. Pure, mammalian expressed human recombinant secreted PHEX (secPHEX), was donated as a kind gift by Dr Philippe Crine and Dr Guy Boileau (Department of Biochemistry, University of Montreal) and was prepared as previously described (Campos, et al., Human recombinant endopeptidase PHEX has a strict S1′ specificity for acidic residues and cleaves peptides derived from fibroblast growth factor-23 and matrix extracellular phosphoglycoprotein, Biochem J 373(Pt 1):271-9, 2003; Boileau, et al., Characterization of PHEX endopeptidase catalytic activity: Identification of parathyroid-hormone-related peptide107-139 as a substrate and osteocalcin, PPi and phosphate as inhibitors, Biochem J 355(Pt 3):707-13, 2001). Briefly, to generate a soluble, secreted form of PHEX, the signal peptide/membrane anchor domain (SA domain) of the protein was transformed into a cleavage-competent signal sequence using a strategy similar to that previously described for NEP (Lemire, et al., Secretion of a type II integral membrane protein induced by mutation of the transmembrane segment, Biochem J 322(Pt 1):335-42, 1997) with modifications for secPHEX as described previously (Campos, et al., supra; Boileau, et al., supra). Purified secPHEX was also analyzed and validated by SDS/PAGE, Western-blotting and N-terminal sequence determined again as described previously (Campos, et al., supra; Boileau, et al., supra ).

Synthetic peptides. Four sets of peptides were synthesized using standard techniques and purchased from MPS Biosystems (Multiple Peptide Systems, Inc. San Diego, Calif.). Peptide purity was greater than 80% via HPLC, ion-exchange and also mass spectrometry. Three of the peptides were derived from MEPE sequence. One of the MEPE-peptides was derivative of the mid-region (residues 238 to 262) of MEPE and contained the RGD-motif (MEPE-RGD). The other two MEPE-peptides consisted of the last 19 COOH-terminal residues encompassing the ASARM-motif (residues 507 to 525). One of the ASARM-peptides was phosphorylated (PO₄-ASARM-peptide) and the other non-phosphorylated (ASARM-peptide). All the peptide sequences are shown in Table 2.

TABLE 2

Sequences and text codes of peptides used for Surface
Plasmon Resonance (SPR) & mineralization studies.

| | | |
|---|---|---|
| Peptide region | Sequence | Code in text |

| | | |
|---|---|---|
| MEPE-ASARM-peptide | RDDSSESSDSG(Sp)S(Sp)E(Sp)DGD | PO₄-ASARM |
| (507-525) | (SEQ ID NO. 14) | peptide |
| MEPE-ASARM-peptide | CFSSRRRDDSSESSDSGSSSESDGD | ASARM-peptide |
| non-phosphorylated | (SEQ ID NO. 5) | |
| (502-525) | | |
| MEPE-RGD mid-region | GSGYTDLQERGDNDISPFSGDGQPF | MEPE-RGD |
| peptide (238-262) | (SEQ ID NO. 15) | |

The MEPE-RGD (mid-region) peptide served as negative control and did not competitively inhibit MEPE-PHEX interactions (see results). The design of an ASARM scrambled peptide was not feasible due to the unique nature of the sequence. Specifically, the key characteristics of the motif are the enriched aspartic acid (D), serine (S) and glutamic acid residues (E), thus, scrambling of the sequence would not substantively alter the key physicochemical properties (i.e., low pI, high charge, acidic-nature and serine-phosphorylation). To overcome this potential pitfall, as mentioned, the control peptide from another region of MEPE (MEPE-RGD) was used as a negative control.

Surface Plasmon Resonance (SPR). A Biacore 3000 SPR instrument (Biacore, Piscataway, N.J.) in conjunction with CM5 research grade chips were used to conduct the Surface Plasmon Resonance (SPR) experiments, as previously described (Morgan, et al., A conserved clathrin assembly motif essential for synaptic vesicle endocytosis, J Neurosci 20(23):8667-8676, 2000; Morgan, et al., Uncoating of clathrin-coated vesicles in presynaptic terminals: Roles for Hsc70 and auxilin, Neuron 32(2):289-300, 200 1; Morgan, et al., Eps15 homology domain-NPF motif interactions regulate clathrin coat assembly during synaptic vesicle recycling, J Biol Chem 278(35):33583-33592, 2003). The experiments were performed at 25° C. using Biacore (Piscataway, N.J.) buffer (10 mM HEPES pH 7.4, 150 mM NaCl, 0.005% surfactant P20) supplemented with 2 mM ZnCl₂ (HBS-P-Zn) or without ZnCl₂ (HBS-P) as indicated. Injected samples also contained 2 mg/mL carboxymethyl-dextran to reduce non-specific association with the dextran matrix of the chip and a flow rate of 5 μL/min was used. The surfaces of research grade CM5 chips were activated by a 6 min injection of a solution containing 0.2M N-ethyl-N9-(dimethylaminopropyl) carbodiimide and 0.05M N-hydroxy-succinimide. MEPE, PHEX and IgG were immobilized on the same chip (ligands) in different individual flow-cells. Each chip contains four separate flow-cells, one cell was left blank as a negative control. The experiments were repeated on two different chips with 3500 RU (response-units) and 6000 RU of immobilized proteins respectively. The response unit or RU is a measure of surface charge density-oscillation and surface-plasmon-positron generation as detected by changes in incident refractive-index monitored by the Biacore optical unit. Specifically, 1000 RU unit equals a change of approximately 1 ng/mm² in surface protein concentration on the chip surface. After immobilization, each surface was blocked by a 6 min injection of 1 M ethanolamine at pH 8.5. Following immobilization, PHEX (analyte) was passed over the surfaces for 6 min at the indicated concentrations in HBS-P-Zn or HBS-P buffer, followed by a 6 min dissociation. Surfaces were then regenerated by a 1 min injection of 6 M guanidine-HCl. Protein-protein interactions between analyte (PHEX) and immobilized ligand (MEPE, PHEX or IgG) are reported as sensorgrams which are plots of RU versus time. An increase in RU reflects changes in the concentration of molecules at the surface of the sensor chip as a result of a specific interaction between analyte and ligand. The following equation was used to calculate EC₅₀ from the Biacore sensorgram: RU=Bₘₐₓ×(secPHEX(μM))/EC₅₀+(secPHEX(μM)). The Bₘₐₓ value indicates the maximal RU (protein-protein interaction) achieved at saturation or high doses of secPHEX.

Surface Plasmon Resonance: Synthetic peptides & competitive PHEX-MEPE binding studies. For these experiments CM-5 chips containing flow-cells immobilized with MEPE, PHEX and IgG ligands at 6000 RU were used. Just prior to SPR injection, stock solution of specific-peptide (as defined for each experiment; see Table 1), and dissolved in analyte buffer (HBS-P—Zn) were added to a constant 250 nM PHEX-analyte solution to give final peptide concentrations of either 0, 11, 22, 42, or 83 nM. In this way a dose dependent inhibition curve was calculated for each of the peptides and apparent K_{D}'s calculated using solution competition (K_{Dapp}). The following equation was used to compute K_{Dapp} values: RU=B_{max/inhib}×(peptide(μM))/K_{Dapp}+(peptide(μM)). The B_{max/inh} value represents the maximal percentage inhibition change in RU (i.e., PHEX-MEPE binding) relative to the “non-peptide” control (0% inhibition of binding), using a constant analyte flow-solution of secPHEX of 250 nM against immobilized MEPE ligand at 6000 RU. Only the phosphorylated ASARM-peptide (PO₄-ASARM-peptide) and the non-phosphorylated ASARM-peptide (ASARM-peptide) were able to inhibit MEPE-PHEX interactions (see results). The control MEPE-RGD peptide had no effect on MEPE-PHEX interactions.

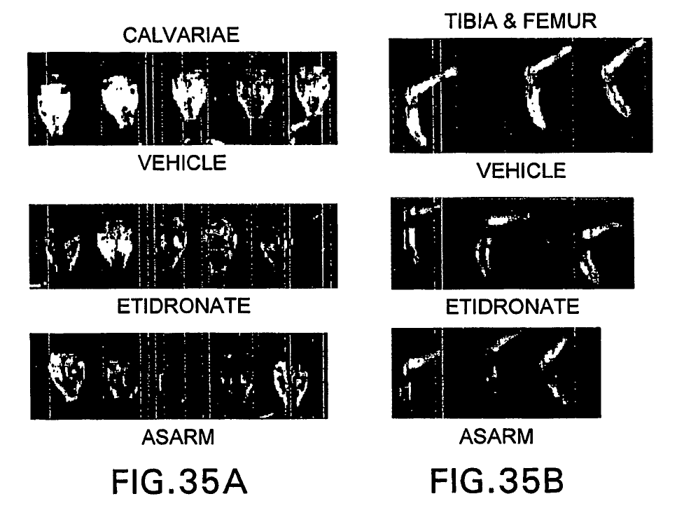

Subcutaneous administration of PO₄-ASARM-peptide, etidronate (EHDP), and calcein. Three separate groups of male ICR Swiss mice (4-5 weeks) were subcutaneously injected once a day (9:00AM), for 12 days except for days 6 and 7 with 50 μL solutions of either (1) Eitidronate (EHDP) (10 mg/kg/day); (2) PO₄-ASARM-peptide (2 mg/kg/day); or (3) vehicle consisting of 5 mM HEPES pH 7.4/150 mM NaCL/0.1% BSA. The synthetic PO₄-ASARM-peptide was dissolved in vehicle (1.0 mg/mL). Etidronate was first dissolved in 150 mM NaCl and then buffered to pH 7.5 with 1M NaOH to give a 5 mg/mL solution. The 10 mg/kg/day doses of etidronate have previously been reported to inhibit mineralization in rats and were used as positive controls (Trechsel, et al., Am J Physiol 232(3):E298-305, 1977; Bonjour, et al., Am J Physiol 229(2):402-8, 1975). All three groups were subcutaneously injected with calcein (20 mg/kg/day) on days 3, 5, 9, and 11. The degree of calcein fluorescence and/or fluorescence-quenching is an indicator of mineralization status (see next section). The calcein injections when given were administered 1 h after the daily injections of PO₄-ASARM-peptide, EHDP or vehicle. Animals were then terminated humanely and calvariae plus femurs/tibia were fixed in 70% ethanol (non-decalcified). After fluorescence imaging (see FIG. 35) calvariae were processed in plastic and stained with Sanderson's stain (see below). Five animals per group were used for statistical analysis of fluorescence quenching and histological Sanderson staining.

Calcein fluorescence in mice calvariae, hind-limbs & fluor-imager analysis of mineralization. A Bio-Rad (Hercules, Calif.) FluorImager^{max} fluorescent imager was used to simultaneously image all the samples from each of the 3 groups (15 animals; five animals per group). This consisted of 10 tibia/femurs and 5 calvariae per group. Samples were placed on top of the FluorImager plate contained within a light-tight box. Ultra-violet light was used to generate calcein-mediated epi-fluorescence and the fluorescent-image was captured on a −40° C., peltier-cooled, 1,340×1040 pixel CCD resolution, CCD digital-camera. Quantitation of fluorescence was carried out using Quantity-1 (Bio-Rad, Hercules, Calif.) fluorescence-software. Pixel saturation was prevented by internal software calibration and exposure adjustment. All fluorescence readings were thus accurately quantitative and non-saturating. Photo-micrographic UV fluorescent analysis of calvarial cross sections embedded in plastic and mounted as slides (<100 micrometer sections) was also undertaken and a description of the fixation and processing is given in the next section.

Sanderson-staining and fluorescence detection of calvarial osteoid & mineralized bone. Staining of non-decalcified calvarial sections was performed using the method described by Sanderson and Bachus (Staining technique to differentiate mineralized and demineralized bone in ground sections, J Histotechnol 20(2): 119-122, 1997). This technique provides differential staining between unmineralized osteoid and mineralized bone. Prior to staining, calvariae were dissected and attached tissue removed. Calvariae were then placed in 70% ethanol for further fixation as described by Sanderson and Bachus (supra). After fixing and processing undecalcified calvariae were embedded in plastic (LR-white acrylic resin (London Resin Co., Reading, England)) as previously described (Garrett, et al., Selective inhibitors of the osteoblast proteasome stimulate bone formation in vivo and in vitro, J Clin Invest 111(11):1771-1782, 2003). Plastic embedded sections were ground to <100 micrometers using a specialized grinding-pestle as described by Donath and Breuner (A method for the study of undecalcified bones and teeth with attached soft tissues. The Sage-Schliff (sawing and grinding) technique, J Oral Pathol 11(4):318-26, 1982). Calcein fluorescence (indicator of mineralization; calcein binds to mineralizing-bone) was then visualized using UV microscopy.

Statistical methods. Differences were assessed statistically by the use of Newman-Keuls or Bonferroni multiple comparison equations (as indicated) after one-way analysis of variance (non-parametric). A P value of less than 0.05 was considered significant. Quantity-1, Bio-Rad (Hercules, Calif.), software was used to analyze the intensity of fluorescence emitted by epi-uv illumination of samples and this was captured by a Bio-Rad (Hercules, Calif.) FluorImager^{max} digital imaging system and data incorporated into GraphPad Prizm-4 software (Graphpad Software. Inc., San Diego, Calif.), for statistical analysis as indicated.

B. Results

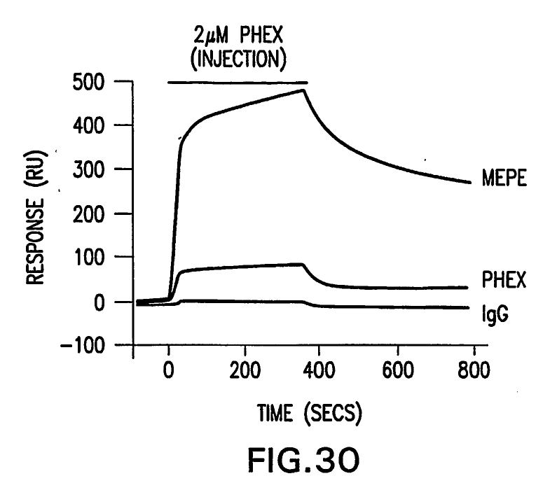

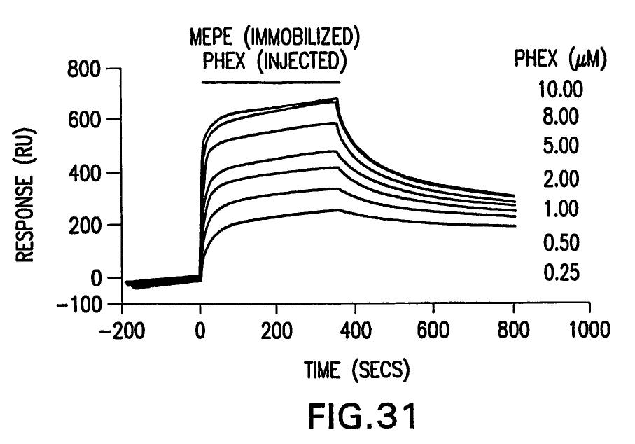

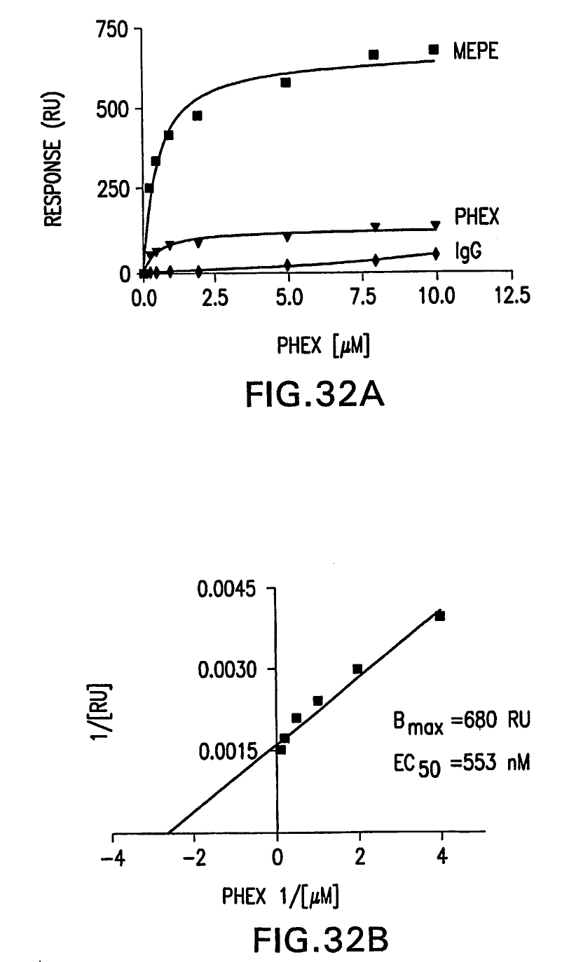

Specific Zn-dependent & dose dependent direct-binding of MEPE to PHEX. FIG. 30 shows a direct protein-protein Zn-dependent interaction between secPHEX and MEPE as monitored and plotted as an SPR sensorgram. A classic protein-association phase was followed by dissociation after the 6 min pulse of secPHEX. There were no significant signals generated between secPHEX and blank activated/blocked chip or control IgG protein. There was a very low-level barely-detectable autologous-interaction between injected secPHEX (analyte) and chip-immobilized secPHEX (ligand). The strong MEPE-PHEX interaction, was dose dependent (see FIG. 31) with an EC₅₀=553 nM (concentration of MEPE required to reach half-maximal binding or RU units). FIGS. 32a and 32b graphically illustrate the dose-dependent PHEX-MEPE binding derived from the Biacore sensorgram shown in FIG. 30. No interaction was observed between MEPE and PHEX in buffer lacking ZnCl₂ (HBS-P) in concentrations of sec PHEX analyte up to 10 μM. Thus, MEPE specifically binds to PHEX in a dose-dependent, Zn-dependent and saturable manner indicative of a ligand-receptor interaction following the law of mass action.

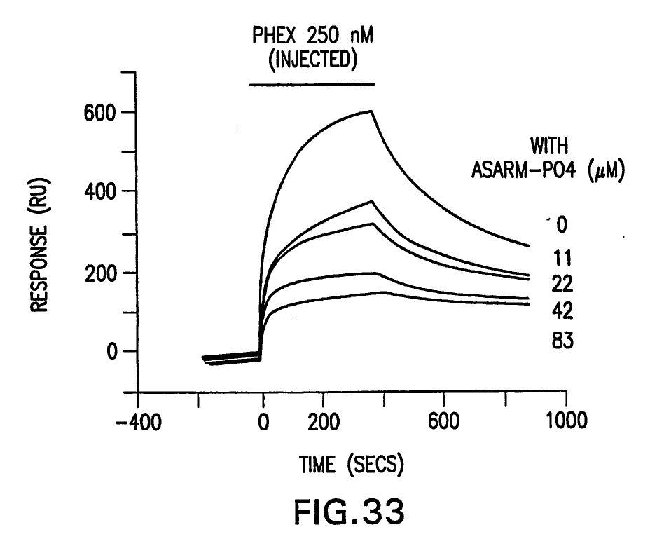

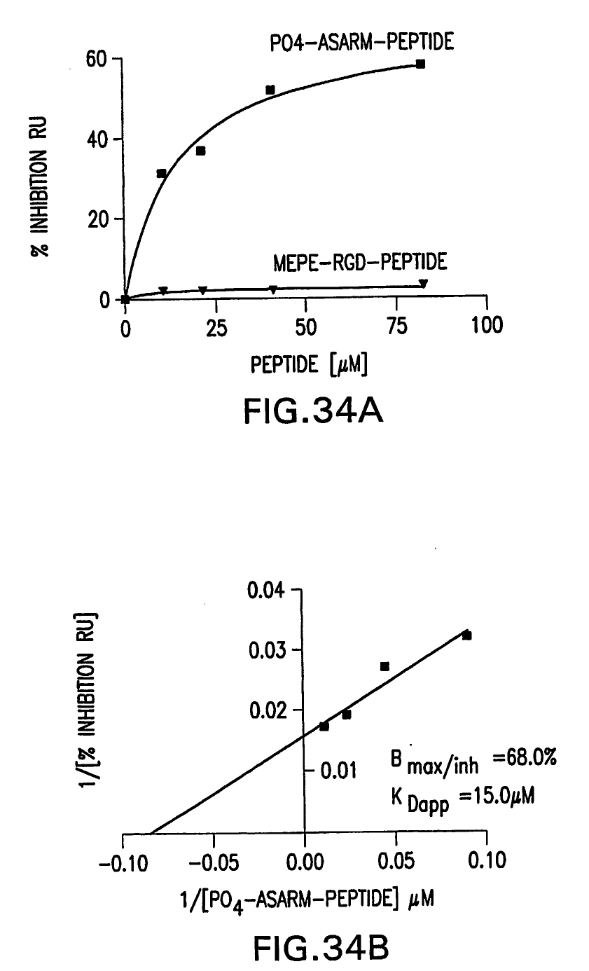

MEPE ASARM-peptides competitively inhibit binding of PHEX to MEPE. Pre-mixing of PO₄-ASARM-peptide or ASARM-peptide with secPHEX prior to injection resulted in a dramatic dose-dependent inhibition of MEPE-PHEX protein-protein interaction (see FIG. 33). The MEPE-PHEX binding also reached saturation at high doses of secPHEX as shown in the SPR sensorgram (FIG. 33). An apparent K_{Dapp} of 15 μM was calculated for the PO₄-ASARM-peptide with a binding maximal inhibition (B_{max/inh}) of 68% (see FIGS. 34a and 34b). The calculated B_{max/inh} value represents the peptide-mediated, maximal percentage-inhibition of MEPE-PHEX binding relative to the non-peptide control (0% inhibition). This value was determined using a constant analyte flow-solution of 250 nM secPHEX against immobilized MEPE ligand at 6000 RU. The unphosphorylated ASARM-peptide was less effective at competitively inhibiting the MEPE-PHEX interaction with an apparent K_{Dapp} of approximately 35 μM compared to 15 μM for PO₄-ASARM-peptide (FIGS. 33 and 34). There was no inhibition of PHEX-MEPE interaction detected at high and low concentrations (up to 85 μM) of control MEPE-RGD-peptide under identical secPHEX pre-mixing conditions.

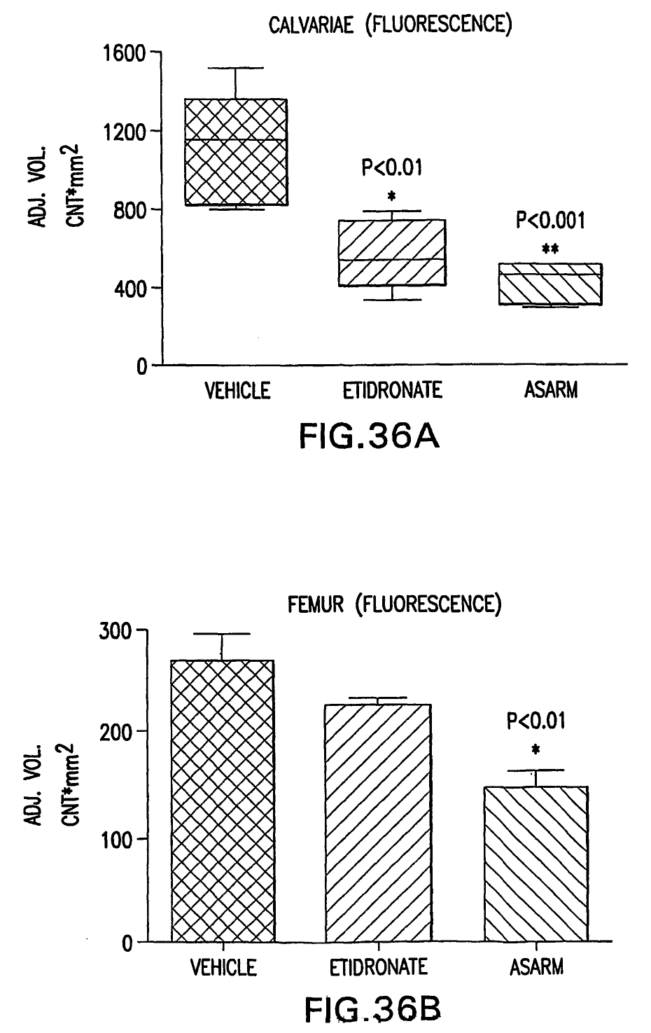

ASARM-peptide & etidronate mediated in-vivo quenching of calcein fluorescence. UV induced calcein epi-fluorescence in dissected calvariae, tibiae and femurs was quenched significantly and markedly in both ASARM-peptide (experimental) and etidronate (positive control) and groups compared to vehicle. FIG. 35 dramatically illustrates the fluorescence-quenching of calvariae and hind-limbs (imaged simultaneously) as captured using a cooled-digital camera (Bio-Rad FluorImager^{max} a system (Hercules, Calif.)). The non-saturated fluorescence was quantitated using Quantity-1 imaging software (Bio-Rad, Hercules, Calif.) and the graphical results for calvariae and tibia/femurs are shown in FIGS. 36a and 36b. A significant drop in calvarial fluorescence of 62% (P<0.001) in PO₄-ASARM-peptide and 48% (P<0.01) in the etidronate group was measured relative to vehicle (see FIGS. 36a and 36b for statistics and graph). A similar marked quenching also occurred with tibia/femur and the PO₄-ASARM-peptide group with fluorescent-intensity reduced by 44.5% (P<0.01) relative to vehicle (FIG. 36b). The quenching of fluorescence in the etidronate group was expected. This is because etidronate potently inhibits mineralization in man and rodents at the levels administered (Trechsel, et al., supra; Bonjour, et al., supra; McCloskey, et al., Comparative effects of intravenous diphosphonates on calcium and skeletal metabolism in man, Bone 8(Suppl 1):S35-41, 1987; McCloskey, et al., Diphosphonates and phosphate homoeostasis in man, Clin Sci (Lond) 74(6):607-612, 1988). The quenching in the ASARM-group was even more marked than the etidronate group in both calvariae and femurs/tibia confirming a major ASARM-peptide inhibition of mineralization in-vivo.

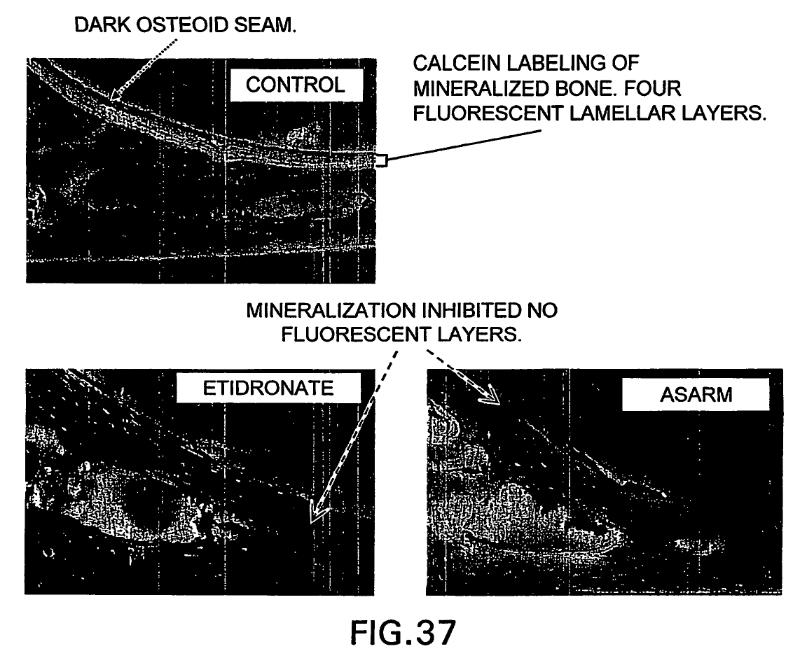

Absence of lamellar fluorescence-bands in calvarial cross-sections of ASARM-peptide & etidronate grouls. FIG. 37 depicts representative UV-fluorescent photo-micrographs of calvarial cross sections from vehicle, etidronate and ASARM-peptide groups. Four distinct lamellar fluorescent layers representing the four separate calcein injections (20 mg/kg/day) are clearly visible in the vehicle group. The fluorescent bands represent calcein-binding to actively mineralizing bone-surface. In contrast, there is a complete absence of these layers in the ASARM-peptide and etidronate treated groups. This strongly suggests that mineralization is inhibited by the addition of ASARM-peptide and by etidronate in-vivo. These results are in agreement with the whole-fluorescence imaging of calvariae and tibiae/femurs described above.

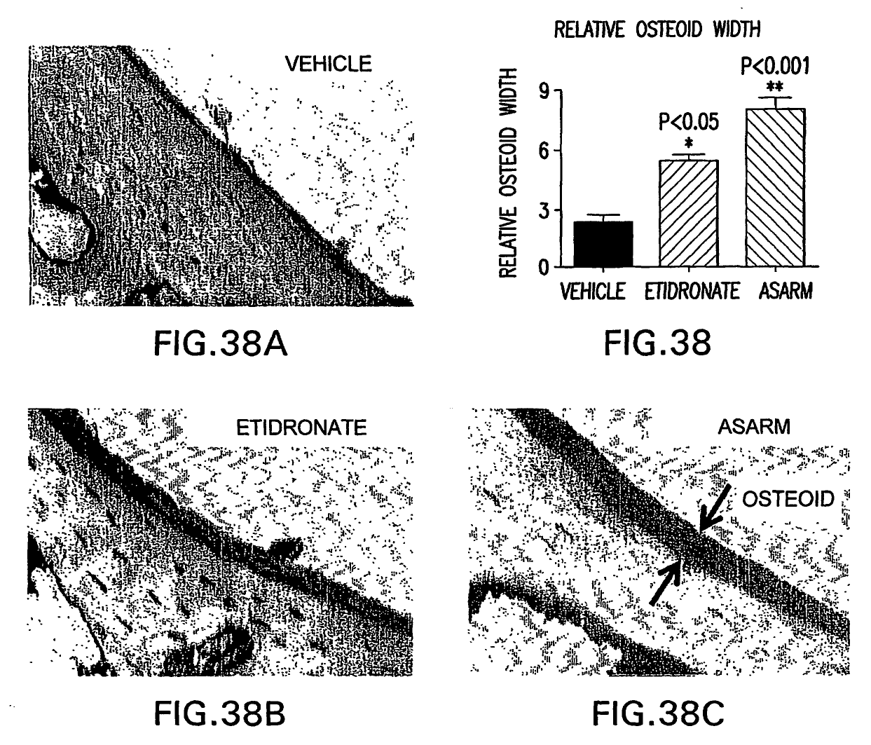

Sanderson's osteoid-staining is increased in calvarial sections of ASARM & etidronate groups. Non-decalcified sections of calvariae from PO₄-ASARM-peptide and etidronate treated groups showed marked increased in osteoid in calvarial cross-sections compared to the vehicle group (FIG. 38). Sanderson histological-staining colors mineralized bone pink and non-mineralized matrix or osteoid blue (Sanderson and Bachus, J Histotechnol 20(2):119-122, 1997). FIG. 38 contains representative photomicrographs of calvarial cross-sections that clearly demonstrate an increased osteoid layer thickness in both ASARM-groups (3.5 fold increase; P<0.001) and the etidronate (2.35 fold increase; P<0.05) relative to vehicle. This increase was statistically significant and confirms inhibition of mineralization by ASARM-peptide groups as well as etidronate. This finding is also consistent with the data described in FIGS. 35 to 37.

### Example VII

### Serum MEPE-ASARM are Elevated in X-Linked Rickets (HYP) and Cause Phosphaturia and Defective Mineralization

A. Materials and Methods

Serum samples. Serum from families with X-linked hypophosphatemic rickets (HYP), from affected and non-affected individuals were previously described and obtained under approved IRB protocols (Frances, et al., HYP-consortium, Nat Genet 11:130-136, 1995; Rowe, et al., Hum Genet 93:291-294, 1994; Rowe, et al., Hum Genet 97: 345-352,1996; Rowe, et al., Hum Mol Genet 6:539-549, 1997). All clinically affected members had fully characterized mutations in the PHEX gene and as previously described were part of a data-set originally used to map and clone the HYP gene. Sera from nine affected HYP patients (three affected males (hemizygous), and six females) and nine normal (five males, four females) were analyzed. Hyp mice were purchased from the JAX laboratories (Bar Harbor, Me.) with the mutation bred into a C57/BL6 background. Standard PCR and southern genotyping was used to confirm hyp phenotypes (Strom, et al., Hum Mol Gent 6(2):165-171, 1997). The sera from six male hyp-mice, three affected (hemizygous) and three normal individuals were used in this study.

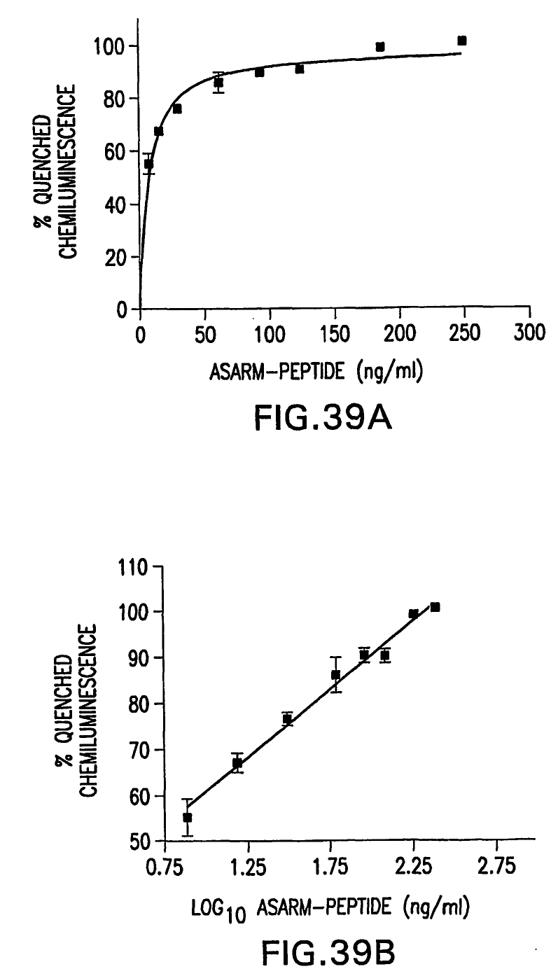

MEPE ASARM-peptide (phosphorylated and non-phosphorylated) & polyclonal antibodies. The carboxy terminal region of MEPE containing residues 507-525 were synthesized using routine techniques by multiple peptide cloning systems (Multiple Peptide Systems, San Diego, Calif.). For ELISA studies, both phosphorylated and non-phosphorylated peptides were synthesized. The phosphorylated-form of the peptide (NH₂—RDDSSESSDSG(Sp)S(Sp)E(Sp)DGD-COOH; SEQ ID NO. 14) was biotinylated using a Pierce EZ-Link Sulfo-NHS-LC-Biotinylation Kit (Pierce & Co, Rockford, Ill.) and the peptide de-salted using an Amersham Biosciences PD-10 Desalting column (Amersham Biosciences, Piscataway, N.J.). Both phosphorylated and non-phosphorylated peptides were equally effective in competition assays and the non-phosphorylated form was routinely used to generate standard curves (see FIGS. 39a and b). Rabbit polyclonal antibodies raised against ASARM-peptide NH₂-CFSSRRRDDSSESSDSGSSSESDGD-COOH (SEQ ID NO. 5) were used in these studies and have been previously described (Rowe, et al., Bone 34:303-319, 2004). Pre-immune sera from the same rabbits were used as negative controls.

ELISA Assay. A new enzyme-linked-immunosorbent-assay (ELISA) was designed to specifically quantitate ASARM-peptide(s) in sera from humans and mice. Components of this assay included 96 well Reacti-Bind Protein-G Coated Plates Pierce & Co, Rockford, Ill.), anti-ASARM-peptide polyclonal antibody (see above), non-phosphorylated ASARM-peptide (also described above), a biotinylated ASARM-peptide (bio-ASARM-peptide), Streptavidin horseradish-peroxidase conjugate (Zymed Laboratories, Inc., San Francisco, Calif.), and an ECL Advance chemiluminescent detection Kit (Amersham BioSciences, Piscataway, N.J.). The overall rationale behind the ELISA was briefly as follows. Initially, the Protein-G-immobilized 96 well plates were used to specifically bind anti-ASARM-IgG antibodies. Synthetic bio-ASARM-peptide was then bound specifically to the immobilized ASARM-antibodies and streptavidin conjugated to horseradish-peroxidase was then allowed to bind to the biotin of the bio-ASARM-peptide. The highly sensitive ECL-enhance chemiluminescence reagent light-emission system (Amersham BioSciences, Piscataway, N.J.) was then used to generate a horseradish-peroxidase catalyzed light-signal. The light-signal was in turn detected by a highly sensitive Bio-Rad FluorImaging system camera (−40° C., peltier-cooled, 1,340×1040 pixel CCD resolution, CCD digital-camera (Bio-Rad, Hercules, Calif.). Quantitation of chemiluminescence was carried out using Quantity-1, Bio-Rad (Hercules, Calif.), imaging-software. Pixel saturation was prevented by internal software-calibration and exposure adjustment. All light-emission readings were thus accurately quantitative and non-saturating. Pre-incubation of bio-ASARM-peptide with increasing amounts of synthetic non-biotinylated ASARM-peptide prior to addition to plates coated with protein-G-anti-ASARM antibody, resulted in a peptide competition for binding to the immobilized anti-ASARM-peptide polyclonal IgG. This in turn resulted in a quenching of the chemiluminescent signal as a function of increasing amounts of non-biotinylated ASARM-peptide in the presence of a constant amount of bio-ASARM-peptide. Maximum chemiluminescence (0% quenching) was achieved in the absence of non-biotinylated peptide (fall bio-ASARM-peptide mediated light-signal emission) and maximum quenching (100%) was achieved in the presence of excess non-biotinylated ASARM-peptide (reduced light emission and binding of bio-ASARM-peptide). Pre-immune serum was also used as a negative control (zero chemiluminescence). Thus, maximum light or 0% quenching indicates low levels of standard synthetic ASARM-peptide or ASARM-peptides in the experimental unknown, and, low-light emission or maximum quenching (100%) is indicative of high concentrations of ASARM-peptide. The following describes the experimental protocol in more detail.

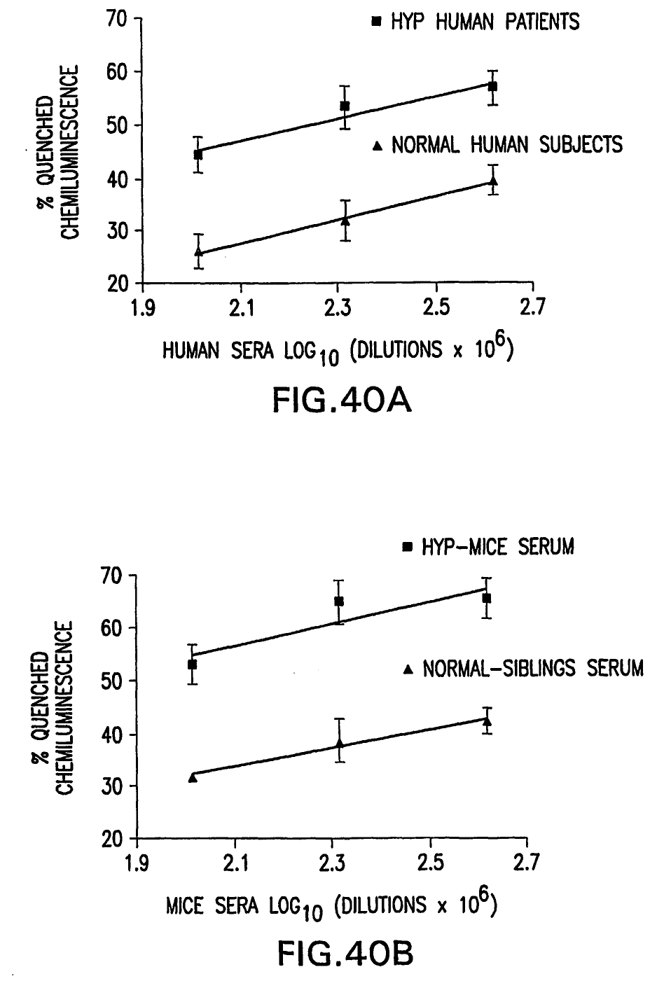

First, to block non-specific binding, 40 μL of Tris-buffered saline (TBS) supplemented with 0.1% (v/v) Tween-20, and 5% non fat dried skimmed milk (TBST-M) was added to each well of a 96 well Protein G coated plates and then incubated at 5° C. with shaking overnight. Plates were then washed three times with the same buffer but without the non-fat dried skimmed milk (TBST). After washing with TBST, 40 μL of a 1:4000 dilution of anti-ASARM antibody was added to each well (diluted in TBST-M). Plates were then incubated at room temperature for 1 hour to facilitate binding of anti-ASARM IgG antibodies to plate-immobilized protein-G. Plates were then washed three times with a TBST. The competition assay was then carried out by separately mixing a constant 0.5 ng/mL concentration of bio-ASARM-peptide with differing concentrations of non-biotinylated ASARM-peptide standards (standard curve) or a range of dilutions of sera as discussed in results and shown FIGS. 39 and 40. All dilutions were made using the TBST buffer. For competition with a 0.5 ng/mL bio-ASARM-peptide the following standard concentrations of non-biotinylated ASARM were optimal: 250, 187, 125, 93, 60, 30, 15 and 7.5 ng/mL (see FIG. 39). Both pre-immune sera and excess non-biotinylated ASARM (1 μg/mL) were used as separate negative controls (zero chemiluminescence). The competition solutions (40 μL) were then added to individual wells of the 96 well plate containing immobilized anti-ASARM-IgG and left for 1 hour at room temperature. Plates were than washed three times with TBST solution. Forty microliters of a 1:20,000 dilution of streptavidin horseradish peroxidase conjugate in TBST was then added to each well and incubated for 25 minutes at room temperature. The plate was washed a further three times with TBST and then directly developed with ECL plus-Advance chemiluminescence kit reagents (Amersham Bioscience, Piscataway, N.J.). The plate was then left to incubate for 5 minutes in the dark and reagents removed from each well prior to chemiluminescence detection by the FluorImager camera (Bio-Rad, Hercules, Calif.). Spiking of sera with non-biotinylated ASARM peptide was also carried out to determine recovery.

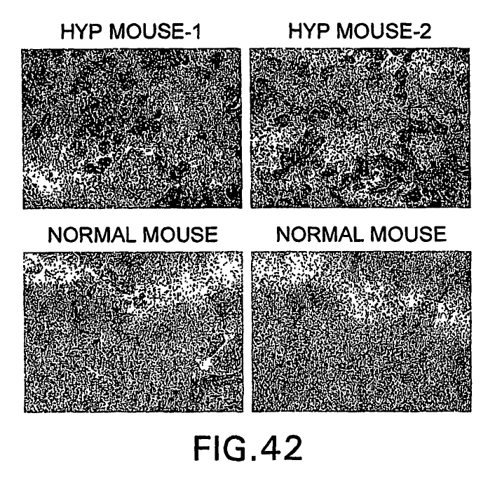

Immunohistochemistry of renal sections using anti-ASARM-peptide polyclonals. Mice (three hyp male mice and three normal male siblings) were first anaesthetized with metofane and cardiac exsanguination used to remove blood for serum-preparation using humane methods and protocols approved by IACUC and UTHSCSA. After exsanguination, left kidneys were then removed from mice and immediately preserved in Millonig's phosphate buffered formalin (UPBF; Medical Industries Inc.). The collected and processed mice sera were used for ELISA analysis as described above. For immunohistochemical detection of ASARM-peptide epitopes, 3 μM thin sections prepared from paraffin-embedded kidneys were incubated with polyclonal anti-ASARM-peptide antibodies raised against the same MEPE C-terminal ASARM-peptide used for ELISA analysis (see above). The immunological reaction was visualized by an ABC alkaline phosphatase kit (Vector, Burlingame, Calif.) and counter-stained with Mayer's hematoxylin-eosin (see FIG. 42).

Statistical methods. Differences were assessed statistically by the use of Newman-Keuls, Bonferroni multiple comparison equations after one-way analysis of variance (non-parametric) or t-tests (as indicated). A P value of less than 0.05 was considered significant. The standard error of the mean (SEM) was used as a representative measure of how far the sample mean differed from the true population mean. Quantity-i Bio-Rad (Hercules, Calif.) software was used to analyze the intensity of chemiluminescent light-emission of samples and this was captured by a Bio-Rad FluorImager^{max} digital imaging system and data incorporated into GraphPad Prizm-4 software (Graphpad Software, Inc., San Diego, Calif.), for statistical analysis.

B. Results

Competitive ELISA using biotinylated and non-biotinylated ASARM-peptides. A competitive ELISA method was used to measure the levels of ASARM-peptide epitopes in serum as described in methods. FIGS. 39a and b show the results derived from standard competition experiments (6 experiments each sample in triplicate) containing 0.5 ng/mL biotinylated peptide and increasing concentrations of non-biotinylated synthetic ASARM-peptide. In FIG. 39a, a one site binding hyperbola clearly demonstrates a classic increased quenching of chemiluminescence correlated with increasing amounts of non-biotinylated ASARM-peptide in the presence of a constant amount (0.5 ng/1 mL) of biotinylated peptide. A K_{D} of 7.5 ng/mL and a Qₘₐₓ (quench maximum) of 98.7% was obtained. A plot of log₁₀ transformation of concentrations against % quenching (FIG. 39b) enabled a linear regression curve to be computed (P<0.0001 and r²=0.9405). The linear range for ASARM-peptide assay was 10-200 ng/mL (4.8 to 95.2 nM). Spiking of serum samples with non-biotinylated ASARM-peptide confirmed a greater than 87% recovery of product.

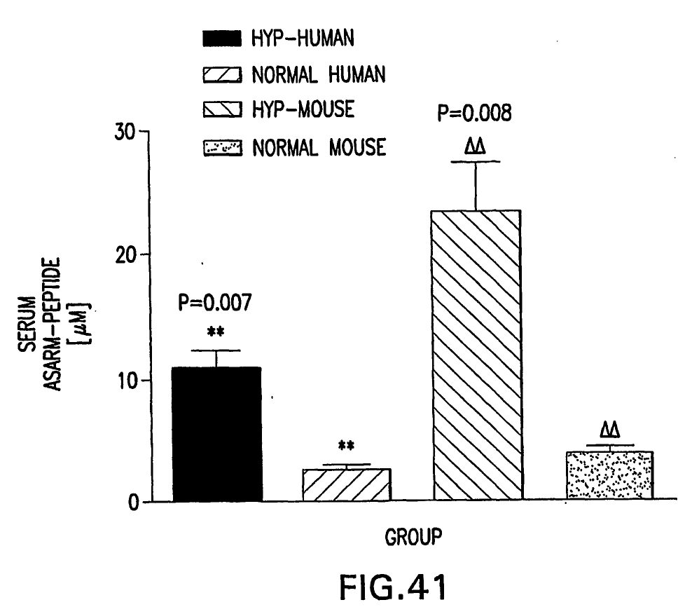

Serum ASARM-peptides in human and mouse X-liked rickets plyp/hyp). FIGS. 40a and b show log₁₀ dilution-transformation linear-regression curves for human HYP patients and hyp mouse serum compared to normals. The linear relationship of quenched-chemiluminescence is arithmetically consistent with the dilution of serum used in each assay. Both human and mouse graphs are remarkably consistent with almost identical slopes (see graph for values). In both the mice and human hyp/HYP subjects a major shift in percentage quenching compared to normals occurs (see FIG. 40). This confirms a substantial increase in serum ASARM-peptide in the HYP/hyp disease state. FIG. 41 illustrates this by comparing the amount of ASARM-peptide in normal and HYP man and mouse. The levels of ASARM-peptide epitope are elevated 5 fold in human subjects affected with HYP (P=0.007) relative to normal subjects (3.25 ρM normal, 15.74 μM HYP). The levels of ASARM-peptide are also dramatically elevated in hyp-mice but even more so with a 6.2 fold increase compared to normal male siblings (3.73 μM and 23.4 μM).

ASARM-peptide epitope elevated binding in hyp renal proximal convoluted tubules. FIG. 42 shows a cross section of hyp mice and normal littermates renal cortex paraffin sections screened with MEPE anti-ASARM polyclonal antibodies. Immuno-positive staining was markedly more pronounced in the hyp renal sections relative to normals. Moreover the staining was localized to areas anatomically consistent with the proximal convolute tubules. The glomeruli are also clearly visible in all sections but were not immuno-positive.

### INCORPORATION BY REFERENCE

Throughout this application, various publications, patents, and/or patent applications are referenced in order to more fully describe the state of the art to which this invention pertains. The disclosures of these publications, patents, and/or patent applications are herein incorporated by reference in their entireties, and for the subject matter for which they are specifically referenced in the same or a prior sentence, to the same extent as if each independent publication, patent, and/or patent application was specifically and individually indicated to be incorporated by reference.

### Other Embodiments

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of treating hyperphosphaternia in a subject, comprising administering an isolated ASARM peptide to the subject using a dosage regimen that decreases serum phosphate levels, thereby treating hyperphosphatemia in the subject.

2. The method of claim 1, wherein the hyperphosphatemia is associated with end stage renal disease, renal osteodystrophy, chronic renal disease, renal toxicity, calcification of kidneys, kidney stones, calcification of arteries, or atherosclerotic lesions.

3. The method of claim 1, wherein at least one serine residue of the isolated ASARM peptide is phosphorylated.

4. The method of claim 3, wherein all serine residues of the isolated ASARM peptide are phosphorylated.

5. A method of treating hypophosphatemia in a subject, wherein the subject displays a normal level of endogenous ASARM peptide, comprising administering an isolated ASARM peptide to the subject using a dosage regimen that increases serum phosphate levels, thereby treating hypophosphatemia in the subject.

6. The method of claim 5, wherein the subject displaying a normal level of endogenous ASARM peptide does not have X-linked hypophosphateric rickets or oncogenic hypophosphatemic osteomalacia.

7. The method of claim 5, wherein the hypophosphatemia is associated with hereditary hypophosphatemic rickets with hypercalciuria, autosomal dominant hypophosphatemic rickets, receptor defect rickets, familial rickets, vitamin-D dependent rickets type-1, defective 25-hydroxylase, Fanconi syndrome, oncogenous syndrome, osteodystrophy, Pagets disease, or metaphyseal dysplasia.

8. The method of claim 5, wherein at least one serine residue of the isolated ASARM peptide is phosphorylated.

9. The method of claim 8, wherein all serine residues of the isolated ASARM peptide are phosphorylated.

10. A method of treating or inhibiting osteoporosis in a subject, comprising administering an isolated ASARM peptide to the subject in a dosage regimen that decreases bone resorption, thereby treating or inhibiting osteoporosis in the subject.

11. A method of treating a subject with a disease involving pathologically elevated levels of endogenous ASARM peptide, comprising inhibiting ASARM peptide activity in the subject, thereby treating the subject with a disease involving pathologically high levels of endogenous ASARM peptide.

12. The method of claim 11, wherein the disease involving pathologically elevated levels of endogenous ASARM peptide is X-linked hypophosphatemic rickets or oncogenic hypophosphatemic osteomalacia.

13. A method of treating or inhibiting ectopic tissue mineralization in a subject, comprising administering ASARM peptide to the subject such that ectopic tissue mineralization is inhibited, thereby treating or inhibiting ectopic tissue mineralization in the subject.

14. The method of claim 13, wherein the subject has ectopic mineralization associated with periodontal disease.

15. The method of claim 13, wherein the subject has ectopic mineralization associated with kidney disease.

16. A method of inhibiting tumor growth in a subject, comprising administering one or more compounds chosen from MEPE, a peptide derived from MEPE, and an isolated ASARM peptide to the subject, thereby inhibiting tumor growth in the subject.

17. The method of claim 16, wherein the subject has a tumor of mesenchymal origin, epithelial origin, endothelial origin, neuroectodermal origin, hematologic cancer, or multiple myeloma.

18. The method of claim 16, wherein at least one serine residue of the isolated ASARM peptide is phosphorylated.

19. The method of claim 18, wherein all serine residues of the isolated ASARM peptide are phosphorylated.

20. The method of claim 16, wherein the isolated ASARM peptide and MEPE are administered.

21. The method of claim 16, wherein the isolated ASARM peptide and the peptide derived from MEPE are administered.

22. The method of claim 16, wherein MEPE and the peptide derived from MEPE are administered.

23. The method of claim 16, wherein MEPE, the peptide derived from MEPE, and the isolated ASARM peptide are administered.

24. A method of preventing cancer cells from metastasizing to bone and/or soft tissues in a subject, comprising administering at least one compound chosen from MEPE, a peptide derived from MEPE, and an isolated ASARM peptide to the subject, thereby preventing cancer cells from metastasizing to bone and/or soft tissue.

25. The method of claim 24, wherein the cancer cells are associated with breast, prostate, lung, kidney, thyroid, multiple myeloma cancers, fibrous dysplasia of bone, oat cell lung carcinomas, haemangiopericytomas, gynaecologic cancers, colon cancer, bladder cancer, cervical cancer, liver cancer, laryngeal cancer and gastrointestinal cancers.

26. The method of claim 24, wherein at least one serine residue of the isolated ASARM peptide is phosphorylated.

27. The method of claim 26, wherein all serine residues of the isolated ASARM peptide are phosphorylated.

28. The method of claim 24, wherein the isolated ASARM peptide and MEPE are administered.

29. The method of claim 24, wherein the isolated ASARM peptide and the peptide derived from MEPE are administered.

30. The method of claim 24, wherein MEPE and the peptide derived from MEPE are administered.

31. The method of claim 24, wherein MEPE, the peptide derived from MEPE, and the isolated ASARM peptide are administered.

32. A method of inhibiting cancer growth and preventing cancer cells from metastasizing to bone and/or soft tissues in a subject, comprising administering at least one compound chosen from MEPE, a peptide derived from MEPE, and an isolated ASARM peptide, thereby preventing cancer cells from metastasizing to bone and/or soft tissue.

33. The method of claim 32, wherein the cancer is associated with breast, prostate, lung, kidney, thyroid, multiple myeloma cancers, fibrous dysplasia of bone, oat cell lung carcinomas, haemangiopericytomas, gynaecologic cancers, colon cancer, bladder cancer, cervical cancer, liver cancer, laryngeal cancer and gastrointestinal cancers.

34. The method of claim 32, wherein at least one serine residue of the isolated ASARM peptide is phosphorylated.

35. The method of claim 32, wherein all serine residues of the isolated ASARM peptide are phosphorylated.

36. The method of claim 32, wherein the isolated ASARM peptide and MEPE are administered.

37. The method of claim 32, wherein the isolated ASARM peptide and the peptide derived from MEPE are administered.

38. The method of claim 32, wherein MEPE and the peptide derived from MEPE are administered.

39. The method of claim 32, wherein MEPE, the peptide derived from MEPE, and the isolated ASARM peptide are administered.

40. A method of identifying a subject with an increased likelihood of having or developing a disease or condition involving abnormal phosphate metabolism, comprising detecting an abnormal level of endogenous ASARM peptide in the subject as compared to a control, wherein an abnormal level of endogenous ASARM peptide in the subject identifies a subject with an increased likelihood of having or developing a disease or condition involving abnormal phosphate metabolism.

41. The method of claim 40, wherein the abnormal phosphate metabolism results in hypophosphatemia.

42. The method of claim 41, wherein the subject has X-linked hypophosphatemic rickets or tumor-induced osteomalacia.

43. The method of claim 40, wherein the abnormal phosphate metabolism is characterized by decreased bone or tooth mineralization or increased bone or tooth resorption compared to a normal subject.

44. The method of claim 43, wherein the abnormal phosphate metabolism is associated with osteoporosis, periodontal disease, or dental caries.

45. The method of claim 40, wherein the subject has hyperphosphatemia.

46. The method of claim 40, wherein the abnormal phosphate metabolism is associated with periodontal disease.

47. A pharmaceutical composition, comprising an isolated ASARM peptide and a pharmaceutically acceptable carrier.

48. The pharmaceutical composition of claim 47, further comprising at least one compound chosen from MEPE and a derivative of MEPE.

49. A method of identifying a compound for treating a disease involving a pathologically elevated level of ASARM peptide, comprising:
(a) contacting a sample comprising an ASARM peptide with the compound, and
(b) detecting a decrease in biological activity of ASARM peptide as compared to a control, whereby an decrease in biological activity of ASARM peptide indicates a compound for treating a disease involving a pathologically elevated level of ASARM peptide.

50. A method of inhibiting MEPE-PHEX binding in a subject, comprising administering an isolated ASARM peptide to the subject in an amount sufficient to inhibit MEPE-PHEX binding.

51. The method of claim 50, wherein at least one serine residue of the ASARM peptide is phosphorylated.

52. The method of claim 51, wherein all serine residues of the ASARM peptide are phosphorylated.


SEQUENCE LISTING (24 sequences)

SEQ ID NO: 1 - protein, 19 aa, Homo sapien
RDDSSESSDSGSSSESDGD

SEQ ID NO: 2 - protein, 18 aa, Mus musculus
RDSSESSSSGSSSESHGD

SEQ ID NO: 3 - protein, 18 aa, Rattus norvegicus
RDSSESSSSGSSSESSGD

SEQ ID NO: 4 - protein, 24 aa, Homo sapien
FSSRRRDDSSESSDSGSSSESDGD

SEQ ID NO: 5 - protein, 25 aa, Homo sapien
CFSSRRRDDSSESSDSGSSSESDGD

SEQ ID NO: 6 - protein, 26 aa, Homo sapien
CGSGYTDLQERGDNDISPFSGDGQPF

SEQ ID NO: 7 - protein, 5 aa, Homo sapien
APTFQ

SEQ ID NO: 8 - protein, 5 aa, Homo sapien
DSESS

SEQ ID NO: 9 - protein, 5 aa, Homo sapien
SSSES

SEQ ID NO: 10 - protein, 15 aa, Homo sapien
APTFQPQTEKTKQSC

SEQ ID NO: 11 - protein, 19 aa, Homo sapien
TDLQERGDNDISPFSGDGQ

SEQ ID NO: 12 - protein, 19 aa, Homo sapien
GRQPHSNRRFSSRRRDDSS

SEQ ID NO: 13 - protein, 18 aa, Homo sapien
DDSSESSDSGSSSESDGD

SEQ ID NO: 14 - protein, 19 aa, Homo sapien
  VARIANT 12, 14, 16: Xaa = a phosphorylated serine
RDDSSESSDSGXSXEXDGD

SEQ ID NO: 15 - protein, 25 aa, Homo sapien
GSGYTDLQERGDNDISPFSGDGQPF

SEQ ID NO: 16 - protein, 19 aa, Macaca fascicularis
REDSSESSDSGSSSESDGD

SEQ ID NO: 17 - protein, 525 aa, Homo sapien
MRVFCVGLLLFSVTWAAPTFQPQTEKTKQSCVEEQRQEEKNKDNIGFHHLGKRINQELSSKENIVQERKKDLSLSEASENKGSSKSQNYFTNRQRLNKEYSISNKENTHNGLRMSIYPKSTGNKGFEDGDDAISKLHDQEEYGAALIRNNMQHIMGPVTAIKLLGEENKENTPRNVLNIIPASMNYAKAHSKDKKKPQRDSQAQKSPVKSKSTHRIQHNIDYLKHLSKVKKIPSDFEGSGYTDLQERGDNDISPFSGDGQPFKDIPGKGEATGPDLEGKDIQTGFAGPSEAESTHLDTKKPGYNEIPEREENGGNTIGTRDETAKEADAVDVSLVEGSNDIMGSTNFKELPGREGNRVDAGSQNAHQGKVEFHYPPAPSKEKRKEGSSDAAESTNYNEIPKNGKGSTRKGVDHSNRNQATLNEKQRFPSKGKSQGLPIPSRGLDNEIKNEMDSFNGPSHENIITHGRKYHYVPHRQNNSTRNKGMPQGKGSWGRQPHSNRRFSSRRRDDSSESSDSGSSSESDGD

SEQ ID NO: 18 - protein, 433 aa, Mus musculus
MQAVSVGLLLFSMTWAAPMPNEDRSSCGNQDSIHKDLAASVYPDPTVDEGTEDGQGALLHPPGQDRYGAALLRNITQPVKSLVTGAELRREGNQEKRPQSVLSVIPADVNDAKVSLKDIKNQESYLLTQSSPVKSKHTKHTRQTRRSTHYLTHLPQIKKTPSDLEGSGSPDLLVRGDNDVPPFSGDGQHFMHIPGKGGAGSGPESSTSRPLSGSSKAEVIDPHMSGLGSNEIPGREGHGGSAYATRDKAAQGAGSAGGSLVGGSNEITGSTNFRELPGKEGNRINAGSQNAHQGKVEFHYPQVASREKVKGGVEHAGRAGYNEIPKSSKGSSSKDAEESKGNQLTLTASQRFPGKGKSQGPALPSHSLSNEVKSEENHYVFHGQNNLTPNKGMSQRRGSWPSRRPNSHRRASTRQRDSSESSSSGSSSESHGD

SEQ ID NO: 19 - protein, 435 aa, Rattus norvegicus
MQAVSVGLFLFSMTWAAPKLNEDGSSGGNQGNIHLASVKPEPMVGKGTEGGRDAPLHLLDQNRQGATLLRNITQPVKSLVTGTEVQSDRNKEKKPQSVLSVIPTDVHNTNDYSEDTENQQRDLLLQNSPGQSKHTPRARRSTHYLTHLPQIRKILSDFEDSASPDLLVRGDNDVPPFSGDGQHFMHTPDRGGAVGSDPESSAGHPVSGSSNVEIVDPHTNGLGSNEIPGREGHIGGAYATRGKTAQGAGSADVSLVEGSNEITGSTKFRELPGKEGNRVDASSQNAHQGKVEFHYPQAPSKEKVKGGSREHTGKAGYNEIPKSSKGGASKDAEESKGNQVTLTESQRFPGKGKGQSSHSLGNEVKSEEDSSNSLSREGIAIAHRRTSHPTRNRGMSQRRGSWASRRPHPHRRVSTRQRDSSESSSSGSSSESSGD

SEQ ID NO: 20 - protein, 555 aa, Macaca fascicularis
MRVFCVGLLFLSVTWAAPTFQPQTEKTKQSCVEEQRITYKGHHEKHGHYVFKCVYMSPGKKNQTDVKQEEKNKDNIGLHHLGKRRYQELSSKENIVQERKKDLSLSEAGENNGSSKSQNYFTNRQRLNKEYSISNKENIHNGLRMSIYPKSTGNKQFADGDDAISELHDQEEYGAALIRNNMQHIMGPVTAIKLLGEENKQSKPKNVLNKIPASMNYAKAHSKDKKKPQRDSQVQKVPVKSKSTHRTQHNIDYPKHLSKVKKIPSDFEGSGYTDLQERGDNDMSPFSGDGQPFKDIPGKGEATGSDLEGKDIQTGFAGPSEAESTNLDTKEPGYNEIPERKENGGNTIGTGDETAKEADAVDVSLVEGNNDIMGSTNFKELPGREGNRVDVGGQNAHQGKVEFHYPPAPSKEKRKEGSSDATESTNYNEIPKNDKGSARKGVDDSNRNQAILHEKQRFPSKGKSQGLPIPSRGLDNEIKTEMDSLNGPSNENIPHSRKYHYVPHRQNNPTRNKGMPHGKGSWGRQPYSNRRLSSRRREDSSESSDSGSSSESDGD

SEQ ID NO: 21 - protein, 165 aa, Homo sapien
  VARIANT 1: Xaa = T or M
  VARIANT 2, 3, 4: Xaa = Any amino acid except Lys
  VARIANT 6: Xaa = Y or S
  VARIANT 11: Xaa - E or G
  VARIANT 13: Xaa = E or K
  VARIANT 14, 15, 16: Xaa = Any amino acid except Lys
  VARIANT (17)...(17): Xaa = G or I
  VARIANT (19)...(22): Xaa = Any amino acid except Lys
  VARIANT (29)...(30): Xaa = Any amino acid except Lys
  VARIANT (80)...(80): Xaa = P or Q
  VARIANT (92)...(99): Xaa = Any amino acid except Lys
  VARIANT (106)...(107): Xaa = Any amino acid except Lys
  VARIANT (110): Xaa = S or G
  VARIANT (111)...(112): Xaa = Any amino acid except Lys
  VARIANT (114)...(117): Xaa = Any amino acid except Lys
XXXXGXNEIPXRXXXXXGXXXXTRDETAXXADAVDVSLVEGSNDIMGSTNFKELPGREGNRVDAGSQNAHQGKVEFHYPXAPSKEKRKEGSXXXXXXXXYNEIPKXXKGXXXKXXXXSNRNQATLNEKQRFPSKGKSQGLPIPSRGLDNEIKNEMDSFNGPSHEN

SEQ ID NO: 22 - protein, 13 aa, Homo sapien
  VARIANT 1: Xaa = Y or S
  VARIANT 6: Xaa = E or G
  VARIANT 8: Xaa = E or K
  VARIANT (9)...(11): Xaa = Any amino acid except Lys
  VARIANT 12: Xaa = G or I
XNEIPXRXXXXXG

SEQ ID NO: 23 - protein, 11 aa, Homo sapien
  VARIANT 7, 8: Xaa = Any amino acid except Lys
  VARIANT 11: Xaa = S or G
YNEIPKXXKGX

SEQ ID NO: 24 - protein, 57 aa, Homo sapien
  VARIANT 46: Xaa = P or Q
DVSLVEGSNDIMGSTNFKELPGREGNRVDAGSQNAHQGKVEFHYPXAPSKEKRKEGS